SEQUENCE LISTING (21 sequences)

SEQ ID NO: 1 - DNA, 2448 bp, Homo sapiens
  source 1..2448: mol_type = genomic DNA
attttgggtggaagcgatagctgagtggcggcggctgctgattgtgttctaggggacggagtaggggaagacgtttgctctcccggaacagcctatctcattcctttctttcgattacccgtggcgcggagagtcagggcggcggctgcggcagcaagggcggcggtggcggcggcggcagctgcagtgacatgtccagcatgaatcccgaatatgattatttattcaagttacttctgattggcgactcaggggttggaaagtcttgccttcttcttaggtttgcagatgatacatatacagaaagctacatcagcacaattggtgtggatttcaaaataagaactatagagttagacgggaaaacaatcaagcttcaaatatgggacacagcaggccaggaaagatttcgaacaatcacctccagttattacagaggagcccatggcatcatagttgtgtatgatgtgacagatcaggagtccttcaataatgttaaacagtggctgcaggaaatagatcgttatgccagtgaaaatgtcaacaaattgttggtagggaacaaatgtgatctgaccacaaagaaagtagtagactacacaacagcgaaggaatttgctgattcccttggaattccgtttttggaaaccagtgctaagaatgcaacgaatgtagaacagtctttcatgacgatggcagctgagattaaaaagcgaatgggtcccggagcaacagctggtggtgctgagaagtccaatgttaaaattcagagcactccagtcaagcagtcaggtggaggttgctgctaaaatttgcctccatccttttctcacagcaatgaatttgcaatctgaacccaagtgaaaaaacaaaattgcctgaattgtactgtatgtagctgcactacaacagattcttaccgtctccacaaaggtcagagattgtaaatggtcaatactgactttttttttattcccttgactcaagacagctaacttcattttcagaactgttttaaacctttgtgtgctggtttataaaataatgtgtgtaatccttgttgctttcctgataccagactgtttcccgtggttggttagaatatattttgttttgatgtttatattggcatgtttagatgtcaggtttagtcttctgaagatgaagttcagccattttgtatcaaacagcacaagcagtgtctgtcactttccatgcataaagtttagtgagatgttatatgtaagatctgatttgctagttcttccttgtagagttataaatggaaagattacactatctgattaatagtttcttcatactctgcatataatttgtggctgcagaatattgtaatttgttgcacactatgtaacaaaacaactgaagatatgtttaataaatattgtacttattggaagtaatatcaaactgtatggtgataagtattgttttgattcttatggttaaagggaaatagagccttgcattatattcaacacagccatttgtgtgtgcacaatgcaaactaaggtattctagacctatcttagagcagcatccagtatttgctttctagataatatgcccaataacatgacctagaggggcttctgtgctgtgtagggatttaaccaacttcagtggttcagggagctcaaactatatgtaaaacaagtttagaatgtatgctatctagcccgttatctctgatccttctctaaaaccatttgaaatagcttcattgatcaacatttcataaatgcatctgtggtagaggtagaaagcagcacctttcctaattggcaaatgatcagactaatgtgtgctaatgtttttcttccatgctttcagtcagattcaactattttatcctccacagttgcttaacttggtgttggaggagggtttaagcattaagataggaagcaggaaatttgattgctctaaatttagaaattatatccctaaaaattaaaacatgaatactgggtggtaatgataattgaggcaaatgtatttattttggtgacattttgcatatatgaagattttctgaaataggaccttcaagatcctagggggttttgtttggtttttaattgtgaggaataaaaaatcttctgcccacactggcattttaaggtgactgaggtcaaacgttgtttccttaggttgaaatagcagccaaaacattcttcacgcaggggcttgggatatggctgctggcaacacattttgttgtgggctccttaatttaatgataaaatttaagctaaacacaagccaaaaatgaataggtttttttaatttttatttttcactaaacaggcaattgaaatacatggtacaaaaataagtggtaagataattgtaaaatgaaatggacagaatattcaattttccatctatgaaaatttcacaataaaaatcatagtttactttgta

SEQ ID NO: 2 - DNA, 618 bp, Homo sapiens
  source 1..618: mol_type = genomic DNA
atgtccagcatgaatcccgaatatgattatttattcaagttacttctgattggcgactcaggggttggaaagtcttgccttcttcttaggtttgcagatgatacatatacagaaagctacatcagcacaattggtgtggatttcaaaataagaactatagagttagacgggaaaacaatcaagcttcaaatatgggacacagcaggccaggaaagatttcgaacaatcacctccagttattacagaggagcccatggcatcatagttgtgtatgatgtgacagatcaggagtccttcaataatgttaaacagtggctgcaggaaatagatcgttatgccagtgaaaatgtcaacaaattgttggtagggaacaaatgtgatctgaccacaaagaaagtagtagactacacaacagcgaaggaatttgctgattcccttggaattccgtttttggaaaccagtgctaagaatgcaacgaatgtagaacagtctttcatgacgatggcagctgagattaaaaagcgaatgggtcccggagcaacagctggtggtgctgagaagtccaatgttaaaattcagagcactccagtcaagcagtcaggtggaggttgctgctaa

SEQ ID NO: 3 - protein, 205 aa, Homo sapiens
MSSMNPEYDYLFKLLLIGDSGVGKSCLLLRFADDTYTESYISTIGVDFKIRTIELDGKTIKLQIWDTAGQERFRTITSSYYRGAHGIIVVYDVTDQESFNNVKQWLQEIDRYASENVNKLLVGNKCDLTTKKVVDYTTAKEFADSLGIPFLETSAKNATNVEQSFMTMAAEIKKRMGPGATAGGAEKSNVKIQSTPVKQSGGGCC

SEQ ID NO: 4 - DNA, 806 bp, Homo sapiens
  source 1..806: mol_type = genomic DNA
gatccaccatgtccagcatgaatcccgaatatgattatttattcaagttacttctgattggcgactcaggggttggaaagaattgccttcttcttaggtttgcagatgatacatatacagaaagctacatcagcacaattggtgtggatttcaaaataagaactatagagttagacgggaaaacaatcaagcttcaaatatgggacacagcaggccaggaaagatttcgaacaatcacctccagttattacagaggagcccatggcatcatagttgtgtatgatgtgacagatcaggagtccttcaataatgttaaacagtggctgcaggaaatagatcgttatgccagtgaaaatgtcaacaaattgttggtagggaacaaatgtgatctgaccacaaagaaagtagtagactacacaacagcgaaggaatttgctgattcccttggaattccgtttttggaaaccagtgctaagaatgcaacgaatgtagaacagtctttcatgacgatggcagctgagattaaaaagcgaatgggtcccggagcaacagctggtggtgctgagaagtccaatgttaaaattcagagcactccagtcaagcagtcaggtggaggttgctgctaactcgacggtaccgcgggcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttac

SEQ ID NO: 5 - DNA, 618 bp, Homo sapiens
  source 1..618: mol_type = genomic DNA
atgtccagcatgaatcccgaatatgattatttattcaagttacttctgattggcgactcaggggttggaaagaattgccttcttcttaggtttgcagatgatacatatacagaaagctacatcagcacaattggtgtggatttcaaaataagaactatagagttagacgggaaaacaatcaagcttcaaatatgggacacagcaggccaggaaagatttcgaacaatcacctccagttattacagaggagcccatggcatcatagttgtgtatgatgtgacagatcaggagtccttcaataatgttaaacagtggctgcaggaaatagatcgttatgccagtgaaaatgtcaacaaattgttggtagggaacaaatgtgatctgaccacaaagaaagtagtagactacacaacagcgaaggaatttgctgattcccttggaattccgtttttggaaaccagtgctaagaatgcaacgaatgtagaacagtctttcatgacgatggcagctgagattaaaaagcgaatgggtcccggagcaacagctggtggtgctgagaagtccaatgttaaaattcagagcactccagtcaagcagtcaggtggaggttgctgctaa

SEQ ID NO: 6 - protein, 205 aa, Homo sapiens
MSSMNPEYDYLFKLLLIGDSGVGKNCLLLRFADDTYTESYISTIGVDFKIRTIELDGKTIKLQIWDTAGQERFRTITSSYYRGAHGIIVVYDVTDQESFNNVKQWLQEIDRYASENVNKLLVGNKCDLTTKKVVDYTTAKEFADSLGIPFLETSAKNATNVEQSFMTMAAEIKKRMGPGATAGGAEKSNVKIQSTPVKQSGGGCC

SEQ ID NO: 7 - DNA, 842 bp, Mus musculus
  source 1..842: mol_type = genomic DNA
gcagagctctctggctactgagaacccactgcttactggcttatcgaaattaatacgactcactatagggagacccaagcttggtaccgagctcggatccactagtaacggccgccagtgtgctggaattcggcttgctgtcgacaccatgggggactacaaggacgacgatgacaaggggggtagcggtggatccagcatgaatcccgaatatgattatttattcaagttacttctgattggcgattctggggttggaaagtcctgccttctccttaggtttgcagatgatacgtatacggaaagctacatcagcacaattggtgtggatttcaagatacgaactatagagttagatgggaaaacaatcaagctacagatatgggacacagcaggccaggaaagatttcgaacaatcacttccagttattacagaggagcccatggcatcatagttgtgtatgatgtgacagatcaggagtccttcaataacgttaaacagtggctgcaggagatagatcgctacgccagtgaaaatgtcaacaagttgttggtagggatcaaatgtgacctgaccacaaagaaagtagtagactacacaacagcaaaggaatttgcagattcccttggaattccatttttggaaaccagtgctaagaacgcaacgaatgtagaacagtctttcatgacgatggcagctgagattaaaaagcgaatgggtcctggagctacagctggtggtgccgagaagtccaatgttaaaatccagagcactccagtcaagcagtcaggtggaggctgctgctaaaatccgcctccgtccttttctcacagcaatgaat

SEQ ID NO: 8 - DNA, 660 bp, Mus musculus
  source 1..660: mol_type = genomic DNA
atgggggactacaaggacgacgatgacaaggggggtagcggtggatccagcatgaatcccgaatatgattatttattcaagttacttctgattggcgattctggggttggaaagtcctgccttctccttaggtttgcagatgatacgtatacggaaagctacatcagcacaattggtgtggatttcaagatacgaactatagagttagatgggaaaacaatcaagctacagatatgggacacagcaggccaggaaagatttcgaacaatcacttccagttattacagaggagcccatggcatcatagttgtgtatgatgtgacagatcaggagtccttcaataacgttaaacagtggctgcaggagatagatcgctacgccagtgaaaatgtcaacaagttgttggtagggatcaaatgtgacctgaccacaaagaaagtagtagactacacaacagcaaaggaatttgcagattcccttggaattccatttttggaaaccagtgctaagaacgcaacgaatgtagaacagtctttcatgacgatggcagctgagattaaaaagcgaatgggtcctggagctacagctggtggtgccgagaagtccaatgttaaaatccagagcactccagtcaagcagtcaggtggaggctgctgctaa

SEQ ID NO: 9 - protein, 219 aa, Mus musculus
MGDYKDDDDKGGSGGSSMNPEYDYLFKLLLIGDSGVGKSCLLLRFADDTYTESYISTIGVDFKIRTIELDGKTIKLQIWDTAGQERFRTITSSYYRGAHGIIVVYDVTDQESFNNVKQWLQEIDRYASENVNKLLVGIKCDLTTKKVVDYTTAKEFADSLGIPFLETSAKNATNVEQSFMTMAAEIKKRMGPGATAGGAEKSNVKIQSTPVKQSGGGCC

SEQ ID NO: 10 - DNA, 806 bp, Homo sapiens
  source 1..806: mol_type = genomic DNA
gatccaccatgtccagcatgaatcccgaatatgattatttattcaagttacttctgattggcgactcaggggttggaaagtcttgccttcttcttaggtttgcagatgatacatatacagaaagctacatcagcacaattggtgtggatttcaaaataagaactatagagttagacgggaaaacaatcaagcttcaaatatgggacacagcaggcctggaaagatttcgaacaatcacctccagttattacagaggagcccatggcatcatagttgtgtatgatgtgacagatcaggagtccttcaataatgttaaacagtggctgcaggaaatagatcgttatgccagtgaaaatgtcaacaaattgttggtagggaacaaatgtgatctgaccacaaagaaagtagtagactacacaacagcgaaggaatttgctgattcccttggaattccgtttttggaaaccagtgctaagaatgcaacgaatgtagaacagtctttcatgacgatggcagctgagattaaaaagcgaatgggtcccggagcaacagctggtggtgctgagaagtccaatgttaaaattcagagcactccagtcaagcagtcaggtggaggttgctgctaactcgacggtaccgcgggcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttac

SEQ ID NO: 11 - DNA, 618 bp, Homo sapiens
  source 1..618: mol_type = genomic DNA
atgtccagcatgaatcccgaatatgattatttattcaagttacttctgattggcgactcaggggttggaaagtcttgccttcttcttaggtttgcagatgatacatatacagaaagctacatcagcacaattggtgtggatttcaaaataagaactatagagttagacgggaaaacaatcaagcttcaaatatgggacacagcaggcctggaaagatttcgaacaatcacctccagttattacagaggagcccatggcatcatagttgtgtatgatgtgacagatcaggagtccttcaataatgttaaacagtggctgcaggaaatagatcgttatgccagtgaaaatgtcaacaaattgttggtagggaacaaatgtgatctgaccacaaagaaagtagtagactacacaacagcgaaggaatttgctgattcccttggaattccgtttttggaaaccagtgctaagaatgcaacgaatgtagaacagtctttcatgacgatggcagctgagattaaaaagcgaatgggtcccggagcaacagctggtggtgctgagaagtccaatgttaaaattcagagcactccagtcaagcagtcaggtggaggttgctgctaa

SEQ ID NO: 12 - protein, 205 aa, Homo sapiens
MSSMNPEYDYLFKLLLIGDSGVGKNCLLLRFADDTYTESYISTIGVDFKIRTIELDGKTIKLQIWDTAGLERFRTITSSYYRGAHGIIVVYDVTDQESFNNVKQWLQEIDRYASENVNKLLVGNKCDLTTKKVVDYTTAKEFADSLGIPFLETSAKNATNVEQSFMTMAAEIKKRMGPGATAGGAEKSNVKIQSTPVKQSGGGCC

SEQ ID NO: 13 - DNA, 806 bp, Homo sapiens
  source 1..806: mol_type = genomic DNA
gatccaccatgtccagcatgaatcccgaatatgattatttattcaagttacttctgattggcgactcaggggttggaaagtcttgccttcttcttaggtttgcagatgatacatatacagaaagctacatcagcacaattggtgtggatttcaaaataagaactatagagttagacgggaaaacaatcaagcttctaatatgggacacagcaggcctggaaagatttcgaacaatcacctccagttattacagaggagcccatggcatcatagttgtgtatgatgtgacagatcaggagtccttcaataatgttaaacagtggctgcaggaaatagatcgttatgccagtgaaaatgtcaacaaattgttggtagggaacaaatgtgatctgaccacaaagaaagtagtagactacacaacagcgaaggaatttgctgattcccttggaattccgtttttggaaaccagtgctaagaatgcaacgaatgtagaacagtctttcatgacgatggcagctgagattaaaaagcgaatgggtcccggagcaacagctggtggtgctgagaagtccaatgttaaaattcagagcactccagtcaagcagtcaggtggaggttgctgctaactcgacggtaccgcgggcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttac

SEQ ID NO: 14 - DNA, 618 bp, Homo sapiens
  source 1..618: mol_type = genomic DNA
atgtccagcatgaatcccgaatatgattatttattcaagttacttctgattggcgactcaggggttggaaagtcttgccttcttcttaggtttgcagatgatacatatacagaaagctacatcagcacaattggtgtggatttcaaaataagaactatagagttagacgggaaaacaatcaagcttctaatatgggacacagcaggcctggaaagatttcgaacaatcacctccagttattacagaggagcccatggcatcatagttgtgtatgatgtgacagatcaggagtccttcaataatgttaaacagtggctgcaggaaatagatcgttatgccagtgaaaatgtcaacaaattgttggtagggaacaaatgtgatctgaccacaaagaaagtagtagactacacaacagcgaaggaatttgctgattcccttggaattccgtttttggaaaccagtgctaagaatgcaacgaatgtagaacagtctttcatgacgatggcagctgagattaaaaagcgaatgggtcccggagcaacagctggtggtgctgagaagtccaatgttaaaattcagagcactccagtcaagcagtcaggtggaggttgctgctaa

SEQ ID NO: 15 - protein, 205 aa, Homo sapiens
MSSMNPEYDYLFKLLLIGDSGVGKSCLLLRFADDTYTESYISTIGVDFKIRTIELDGKTIKLLIWDTAGQERFRTITSSYYRGAHGIIVVYDVTDQESFNNVKQWLQEIDRYASENVNKLLVGNKCDLTTKKVVDYTTAKEFADSLGIPFLETSAKNATNVEQSFMTMAAEIKKRMGPGATAGGAEKSNVKIQSTPVKQSGGGCC

SEQ ID NO: 16 - DNA, 806 bp, Homo sapiens
  source 1..806: mol_type = genomic DNA
gatccaccatgtccagcatgaatcccgaatatgattatttattcaagttacttctgattggcgactcaggggttggaaagtcttgccttcttcttaggtttgcagatgatacatatacagaaagctacatcagcacaattggtgtgaacttcaaaataagaactatagagttagacgggaaaacaatcaagcttcaaatatgggacacagcaggccaggaaagatttcgaacaatcacctccagttattacagaggagcccatggcatcatagttgtgtatgatgtgacagatcaggagtccttcaataatgttaaacagtggctgcaggaaatagatcgttatgccagtgaaaatgtcaacaaattgttggtagggaacaaatgtgatctgaccacaaagaaagtagtagactacacaacagcgaaggaatttgctgattcccttggaattccgtttttggaaaccagtgctaagaatgcaacgaatgtagaacagtctttcatgacgatggcagctgagattaaaaagcgaatgggtcccggagcaacagctggtggtgctgagaagtccaatgttaaaattcagagcactccagtcaagcagtcaggtggaggttgctgctaactcgacggtaccgcgggcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttac

SEQ ID NO: 17 - DNA, 618 bp, Homo sapiens
  source 1..618: mol_type = genomic DNA
atgtccagcatgaatcccgaatatgattatttattcaagttacttctgattggcgactcaggggttggaaagtcttgccttcttcttaggtttgcagatgatacatatacagaaagctacatcagcacaattggtgtgaacttcaaaataagaactatagagttagacgggaaaacaatcaagcttcaaatatgggacacagcaggccaggaaagatttcgaacaatcacctccagttattacagaggagcccatggcatcatagttgtgtatgatgtgacagatcaggagtccttcaataatgttaaacagtggctgcaggaaatagatcgttatgccagtgaaaatgtcaacaaattgttggtagggaacaaatgtgatctgaccacaaagaaagtagtagactacacaacagcgaaggaatttgctgattcccttggaattccgtttttggaaaccagtgctaagaatgcaacgaatgtagaacagtctttcatgacgatggcagctgagattaaaaagcgaatgggtcccggagcaacagctggtggtgctgagaagtccaatgttaaaattcagagcactccagtcaagcagtcaggtggaggttgctgctaa

SEQ ID NO: 18 - protein, 205 aa, Homo sapiens
MSSMNPEYDYLFKLLLIGDSGVGKSCLLLRFADDTYTESYISTIGVNFKIRTIELDGKTIKLQIWDTAGQERFRTITSSYYRGAHGIIVVYDVTDQESFNNVKQWLQEIDRYASENVNKLLVGNKCDLTTKKVVDYTTAKEFADSLGIPFLETSAKNATNVEQSFMTMAAEIKKRMGPGATAGGAEKSNVKIQSTPVKQSGGGCC

SEQ ID NO: 19 - DNA, 2268 bp, synthetic construct
  source 1..2268: mol_type = other DNA
atggaagacgccaaaaacataaagaaaggcccggcgccattctatccgctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaactcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggataccaggtggcccccgctgaattggagtcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgccaaaaagttgcgcggaggagttgtgtttgtggacgaagtaccgaaaggtcttaccggaaaactcgacgcaagaaaaatcagagagatcctcataaaggccaagaagggcggaaagtccaaattgatgtccagcatgaatcccgaatatgattatttattcaagttacttctgattggcgactcaggggttggaaagaattgccttcttcttaggtttgcagatgatacatatacagaaagctacatcagcacaattggtgtggatttcaaaataagaactatagagttagacgggaaaacaatcaagcttcaaatatgggacacagcaggccaggaaagatttcgaacaatcacctccagttattacagaggagcccatggcatcatagttgtgtatgatgtgacagatcaggagtccttcaataatgttaaacagtggctgcaggaaatagatcgttatgccagtgaaaatgtcaacaaattgttggtagggaacaaatgtgatctgaccacaaagaaagtagtagactacacaacagcgaaggaatttgctgattcccttggaattccgtttttggaaaccagtgctaagaatgcaacgaatgtagaacagtctttcatgacgatggcagctgagattaaaaagcgaatgggtcccggagcaacagctggtggtgctgagaagtccaatgttaaaattcagagcactccagtcaagcagtcaggtggaggttgctgctaa

SEQ ID NO: 20 - RNA, 2268 nt, synthetic construct
  source 1..2268: mol_type = other RNA
atggaagacgccaaaaacataaagaaaggcccggcgccattctatccgctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaactcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggataccaggtggcccccgctgaattggagtcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgccaaaaagttgcgcggaggagttgtgtttgtggacgaagtaccgaaaggtcttaccggaaaactcgacgcaagaaaaatcagagagatcctcataaaggccaagaagggcggaaagtccaaattgatgtccagcatgaatcccgaatatgattatttattcaagttacttctgattggcgactcaggggttggaaagaattgccttcttcttaggtttgcagatgatacatatacagaaagctacatcagcacaattggtgtggatttcaaaataagaactatagagttagacgggaaaacaatcaagcttcaaatatgggacacagcaggccaggaaagatttcgaacaatcacctccagttattacagaggagcccatggcatcatagttgtgtatgatgtgacagatcaggagtccttcaataatgttaaacagtggctgcaggaaatagatcgttatgccagtgaaaatgtcaacaaattgttggtagggaacaaatgtgatctgaccacaaagaaagtagtagactacacaacagcgaaggaatttgctgattcccttggaattccgtttttggaaaccagtgctaagaatgcaacgaatgtagaacagtctttcatgacgatggcagctgagattaaaaagcgaatgggtcccggagcaacagctggtggtgctgagaagtccaatgttaaaattcagagcactccagtcaagcagtcaggtggaggttgctgctaa

SEQ ID NO: 21 - protein, 755 aa, synthetic construct
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKLMSSMNPEYDYLFKLLLIGDSGVGKNCLLLRFADDTYTESYISTIGVDFKIRTIELDGKTIKLQIWDTAGQERFRTITSSYYRGAHGIIVVYDVTDQESFNNVKQWLQEIDRYASENVNKLLVGNKCDLTTKKVVDYTTAKEFADSLGIPFLETSAKNATNVEQSFMTMAAEIKKRMGPGATAGGAEKSNVKIQSTPVKQSGGGCC

(19) United States
(12) Patent Application Publication
(10) Pub. No.: US 2026/0209725 A1
(43) Pub. Date: Jul. 23, 2026

(54) COMPOSITIONS AND METHODS FOR TREATING PARKINSON'S DISEASE

(71) Applicants: MOTIGENIX SINGAPORE PTE. LTD., Singapore (SG); RELIABLE HOLDINGS CO., LTD., Eden Island (SC)

(72) Inventors: Mohsen Amir ALIPOUR, Ottawa, Ontario (CA); Kuo-Tang TSENG, Taipei City (TW); Zemin YAO, Orleans, Ontario (CA)

(21) Appl. No.: 19/143,410

(22) PCT Filed: Dec. 21, 2023
(86) PCT No.: PCT/SG2023/050850

§ 371 Date: Jun. 26, 2025

Related U.S. Application Data
(60) Provisional application No. 63/477,237, filed on Dec. 27, 2022.

(51) Int. Cl. C12N 9/14; A61K 9/51; A61K 38/00; A61P 25/16

(52) U.S. Cl. CPC C12N 9/14; A61K 9/5123; A61P 25/16; A61K 38/00; C12Y 306/05002

(57) ABSTRACT

Compounds, compositions, uses, and methods for increasing cell viability of a dopaminergic neuron, or for preventing or treating dopaminergic neuronal death, are provided herein. In certain examples, methods for reducing symptoms and/or for preventing or treating Parkinson's disease in a subject in need thereof are provided which may include a step of treatment with a GDP-bound form of Rab1a (Rab1a^{GDP}), one or more expressible nucleic acids encoding Rab1a^{GDP}, or a combination thereof.

### FIELD OF INVENTION

The present invention relates generally to treatment of Parkinson's disease. More specifically, the present invention relates to compositions and methods for enhancing microautophagy to treat various forms of Parkinson's disease and related diseases or disorders.

### BACKGROUND

There are three known cellular processes, in eukaryotes, through which the cytosolic content and/or subcellular organelles can be delivered to or acquired by lysosomes for degradation: namely macroautophagy (commonly known as autophagy), microautophagy, and chaperone-mediated autophagy. Microautophagy, unlike macroautophagy or chaperone-mediated autophagy, is a type of autophagic process that is mediated by direct lysosomal (in mammals) or vacuolar (in plants and fungi) piecemeal engulfment of a cytoplasmic target or cargo (e.g. protein, lipid, glycogen or pathogen).

Microautophagy may contribute to cytosolic protein degradation via late endosomes (MVBs). In addition, microautophagy may also support direct glycogen delivery to lysosomes and its degradation. In this regard, it is contemplated that malfunctioning or insufficient microautophagy may be associated with development of a variety of metabolic and/or neurological diseases, for example.

Clearly, accumulation of particular proteins, lipids, and/or glycogens may be associated with a variety of important diseases, disorders, and conditions. Methods for targeting such proteins, lipids, and/or glycogens are desirable.

Parkinson's disease (PD) is the second most common neurodegenerative disease after Alzheimer's disease (Zheng et al., 2018). PD a progressive, neurodegenerative disorder characterized by severe motor symptoms, including static tremor, postural imbalance, bradykinesia and muscle rigidity. Although some individuals inherit genetic mutations that will lead to a familial form of Parkinson's disease, the majority of cases are sporadic (Schapira, 2008). Unfortunately, the ultimate cause of PD remains poorly understood. PD Studies of the mutations that cause familial PD, and postmortem brain studies of idiopathic, sporadic PD, have highlighted mitochondrial dysfunction, oxidative stress, and protein metabolism by the ubiquitin-proteasomal and autophagy systems as being central to the pathogenesis (Schapira & Tolosa, 2010).

α-synuclein, encoded by the SNCA gene, is a neuronal protein that regulates synaptic vesicle trafficking and subsequent neurotransmitter release. One of the hallmarks of Parkinson's disease is the presence of Lewy bodies characterized by aggregates of α-synuclein forming insoluble fibrils in presynaptic dopaminergic neurons of the midbrain, particularly the substantia nigra (Zeng et al., 2018). Strategies have therefore been developed for treating synucleinopathies by developing compounds that inhibit aggregation of alpha-synuclein, albeit with limited success. Indeed, since the introduction of levodopa (L-Dopa) in the 1960s, there have been relatively few developments in the treatment of PD as there are no disease-modifying treatments and management of the disease predominantly consists of dopaminergic drugs (Stoker et al., 2018). Unfortunately, the chronic use of L-Dopa results in significant adverse effects such as dyskinesias, hallucinations and impulse control disorder.

Alternative, additional, and/or improved anti-Parkinson agents, compositions, and/or methods for treating Parkinson's disease and related diseases or disorders are desirable.

### SUMMARY OF INVENTION

As described in detail herein, it has now been identified that treatment with a microautophagy-enhancing agent comprising a GDP-bound form of Rab1a, such as Rab1a^{S25N}, Rab1a^{N124I}, Rab1a^{D41N}, Rab1a^{D47N}, or another dominant negative (DN) GDP-bound form of Rab1a, or one or more expressible nucleic acids encoding such a Rab1a^{GDP}, may be used to decrease cell death and/or increase cell viability of cells in a subject affected by Parkinson's disease, thereby reducing symptoms of Parkinson's disease in the subject.

In an embodiment, there is provided herein a method for reducing symptoms of Parkinson's disease, or for preventing or treating symptoms of Parkinson's disease, in a subject in need thereof, said method comprising:
treating the subject with a GDP-bound form of Rab1a (Rab1a^{GDP}), one or more expressible nucleic acids encoding Rab1a^{GDP}, or a combination thereof;
thereby increasing cellular levels of Rab1a^{GDP} in the subject, resulting in decreased symptoms of Parkinson's disease in the subject.

In an embodiment, the symptoms of Parkinson's disease include at least one of the following: static tremor, postural imbalance, bradykinesia, muscle rigidity, punding, dyskinesias, hallucinations, impulse control disorder and sleep disorders.

In another embodiment, there is provided herein a method for increasing cell viability of a dopaminergic neuron in vitro or in vivo, said method comprising:
treating the cell with a GDP-bound form of Rab1a (Rab1a^{GDP}), one or more expressible nucleic acids encoding Rab1a^{GDP}, or a combination thereof;
thereby increasing a cellular level of Rab1a^{GDP} in the dopaminergic neuron, resulting in increased cell viability of the dopaminergic neuron.

In another embodiment of any of the above method or methods, the Rab1a^{GDP} may be or may comprise Rab1a^{S25N}, Rab1a^{N124I}, Rab1a^{D41N}, Rab1a^{D47N}, or another dominant negative (DN) GDP-bound form of Rab1a.

In still another embodiment of any of the above method or methods, the Rab1a^{GDP} may comprise the amino acid sequence:

(SEQ ID NO: 6; Human Rab1a^{S25N})
MSSMNPEYDYLFKLLLIGDSGVGKNCLLLRFADDTYTESYISTIGVDFKIRTIELDGK
TIKLQIWDTAGQERFRTITSSYYRGAHGIIVVYDVTDQESFNNVKQWLQEIDRYASE
NVNKLLVGNKCDLTTKKVVDYTTAKEFADSLGIPFLETSAKNATNVEQSFMTMAAE
IKKRMGPGATAGGAEKSNVKIQSTPVKQSGGGCC;
(SEQ ID NO: 9; Mouse Rab1^{N124I})
MGDYKDDDDKGGSGGSSMNPEYDYLFKLLLIGDSGVGKSCLLLRFADDTYTESYIS
TIGVDFKIRTIELDGKTIKLQIWDTAGQERFRTITSSYYRGAHGIIVVYDVTDQESFNN
VKQWLQEIDRYASENVNKLLVGIKCDLTTKKVVDYTTAKEFADSLGIPFLETSAKNA
TNVEQSFMTMAAEIKKRMGPGATAGGAEKSNVKIQSTPVKQSGGGCC;
the amino acid sequence of human Rab1a^{D41N};
or
(SEQ ID NO: 18; Human Rab1a^{D47N})
MSSMNPEYDYLFKLLLIGDSGVGKSCLLLRFADDTYTESYISTIGVNFKIRTIELDGKT
IKLQIWDTAGQERFRTITSSYYRGAHGIIVVYDVTDQESFNNVKQWLQEIDRYASEN
VNKLLVGNKCDLTTKKVVDYTTAKEFADSLGIPFLETSAKNATNVEQSFMTMAAEI
KKRMGPGATAGGAEKSNVKIQSTPVKQSGGGCC;

or a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity with any of these sequences and preferentially binding GDP.

In yet another embodiment of any of the above method or methods, the Rab1a^{GDP} may consist of the amino acid sequence:

(SEQ ID NO: 6; Human Rab1a^{S25N})
MSSMNPEYDYLFKLLLIGDSGVGKNCLLLRFADDTYTESYISTIGVDFKIRTIELDGK
TIKLQIWDTAGQERFRTITSSYYRGAHGIIVVYDVTDQESFNNVKQWLQEIDRYASE
NVNKLLVGNKCDLTTKKVVDYTTAKEFADSLGIPFLETSAKNATNVEQSFMTMAAE
IKKRMGPGATAGGAEKSNVKIQSTPVKQSGGGCC;
(SEQ ID NO: 9; Mouse Rab1^{N124I})
MGDYKDDDDKGGSGGSSMNPEYDYLFKLLLIGDSGVGKSCLLLRFADDTYTESYIS
TIGVDFKIRTIELDGKTIKLQIWDTAGQERFRTITSSYYRGAHGIIVVYDVTDQESENN
VKQWLQEIDRYASENVNKLLVGIKCDLTTKKVVDYTTAKEFADSLGIPFLETSAKNA
TNVEQSFMTMAAEIKKRMGPGATAGGAEKSNVKIQSTPVKQSGGGCC;
the amino acid sequence of human Rab1a^{D41N});
or
(SEQ ID NO: 18; Human Rab1a^{D47N})
MSSMNPEYDYLFKLLLIGDSGVGKSCLLLRFADDTYTESYISTIGVNFKIRTIELDGKT
IKLQIWDTAGQERFRTITSSYYRGAHGIIVVYDVTDQESFNNVKQWLQEIDRYASEN
VNKLLVGNKCDLTTKKVVDYTTAKEFADSLGIPFLETSAKNATNVEQSFMTMAAEI
KKRMGPGATAGGAEKSNVKIQSTPVKQSGGGCC;

or a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity with any of these sequences and preferentially binding GDP.

In another embodiment of any of the above method or methods, the Rab1a^{GDP} may comprise or consist of the amino acid sequence:

(SEQ ID NO: 6; Human Rab1a^{S25N})
MSSMNPEYDYLFKLLLIGDSGVGKNCLLLRFADDTYTESYISTIGVDFK
IRTIELDGKTIKLQIWDTAGQERFRTITSSYYRGAHGIIVVYDVTDQES
FNNVKQWLQEIDRYASENVNKLLVGNKCDLTTKKVVDYTTAKEFADSLG
IPFLETSAKNATNVEQSFMTMAAEIKKRMGPGATAGGAEKSNVKIQSTP
VKQSGGGCC;

or a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity therewith and preferentially binding GDP.

In still another embodiment of any of the above method or methods, the Rab1a^{GDP} may be in the form of a fusion protein, wherein the Rab1a^{GDP} is fused or otherwise directly or indirectly linked, optionally via a linker, with a signaling or targeting peptide, a fluorescent peptide or other marker or tracer, or another peptide or non-peptide moiety for targeted delivery, facilitating cell uptake, increasing stability or in vivo half-life, or improving another therapeutic, diagnostic, or in vivo property of the Rab1a^{GDP}.

In yet another embodiment of any of the above method or methods, the fusion protein may comprise the amino acid sequence:

(SEQ ID NO: 6; Human Rab1a^{S25N})
MSSMNPEYDYLFKLLLIGDSGVGKNCLLLRFADDTYTESYISTIGVDFK
IRTIELDGKTIKLQIWDTAGQERFRTITSSYYRGAHGIIVVYDVTDQES
FNNVKQWLQEIDRYASENVNKLLVGNKCDLTTKKVVDYTTAKEFADSLG
IPFLETSAKNATNVEQSFMTMAAEIKKRMGPGATAGGAEKSNVKIQSTP
VKQSGGGCC;

or a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity therewith and preferentially binding GDP.

In another embodiment of any of the above method or methods, the Rab1a^{GDP} may be in the form of a fusion protein, and may comprise the amino acid sequence:

(SEQ ID NO: 21)
MEDAKNIKKGPAPFYPLEDGTAGEQLHKAMKRYALVPGTIAFTDAHIEV
NITYAEYFEMSVRLAEAMKRYGLNTNHRIVVCSENSLQFFMPVLGALFI
GVAVAPANDIYNERELLNSMNISQPTVVFVSKKGLQKILNVQKKLPIIQ
KIIIMDSKTDYQGFQSMYTFVTSHLPPGFNEYDFVPESFDRDKTIALIM
NSSGSTGLPKGVALPHRTACVRFSHARDPIFGNQIIPDTAILSVVPFHH
GFGMFTTLGYLICGFRVVLMYRFEEELFLRSLQDYKIQSALLVPTLFSF
FAKSTLIDKYDLSNLHEIASGGAPLSKEVGEAVAKRFHLPGIRQGYGLT
ETTSAILITPEGDDKPGAVGKVVPFFEAKVVDLDTGKTLGVNQRGELCV
RGPMIMSGYVNNPEATNALIDKDGWLHSGDIAYWDEDEHFFIVDRLKSL
IKYKGYQVAPAELESILLQHPNIFDAGVAGLPDDDAGELPAAVVVLEHG
KTMTEKEIVDYVASQVTTAKKLRGGVVFVDEVPKGLTGKLDARKIREIL
IKAKKGGKSKLMSSMNPEYDYLFKLLLIGDSGVGKNCLLLRFADDTYTE
SYISTIGVDFKIRTIELDGKTIKLQIWDTAGQERFRTITSSYYRGAHGI
IVVYDVTDQESFNNVKQWLQEIDRYASENVNKLLVGNKCDLTTKKVVDY
TTAKEFADSLGIPFLETSAKNATNVEQSFMTMAAEIKKRMGPGATAGGA
EKSNVKIQSTPVKQSGGGCC;

or a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity therewith and preferentially binding GDP.

In still another embodiment of any of the above method or methods, the one or more expressible nucleic acids may encode any one or more of the Rab1a^{GDP}s as defined herein.

In yet another embodiment of any of the above method or methods, the one or more expressible nucleic acids may be DNA-based, or RNA-based.

In another embodiment of any of the above method or methods, the one or more expressible nucleic acids may transiently express the Rab1a^{GDP} in the subject, or wherein the one or more expressible nucleic acids may integrate in the subject genome and express the Rab1a^{GDP} in the subject.

In another embodiment of any of the above method or methods, the one or more expressible nucleic acids may transiently express the Rab1a^{GDP} in the dopaminergic neuron, or wherein the one or more expressible nucleic acids may integrate in the cell genome and express the Rab1a^{GDP} in the dopaminergic neuron.

In still another embodiment of any of the above method or methods, the one or more expressible nucleic acids may comprise one or more expression vectors, plasmids, or mRNAs encoding and capable of expressing the Rab1a^{GDP} inside the subject.

In still another embodiment of any of the above method or methods, the one or more expressible nucleic acids may comprise one or more expression vectors, plasmids, or mRNAs encoding and capable of expressing the Rab1a^{GDP} inside the cell.

In yet another embodiment of any of the above method or methods, the one or more expressible nucleic acids may comprise a nucleic acid sequence of:

(Human Rab1a^{S25N} ORF Codon Sequence, SEQ ID NO: 5)
ATGTCCAGCATGAATCCCGAATATGATTATTTATTCAAGTTACTTCTGATTGGCG
ACTCAGGGGTTGGAAAGAATTGCCTTCTTCTTAGGTTTGCAGATGATACATATAC
AGAAAGCTACATCAGCACAATTGGTGTGGATTTCAAAATAAGAACTATAGAGTT
AGACGGGAAAACAATCAAGCTTCAAATATGGGACACAGCAGGCCAGGAAAGAT
TTCGAACAATCACCTCCAGTTATTACAGAGGAGCCCATGGCATCATAGTTGTGTA
TGATGTGACAGATCAGGAGTCCTTCAATAATGTTAAACAGTGGCTGCAGGAAAT
AGATCGTTATGCCAGTGAAAATGTCAACAAATTGTTGGTAGGGAACAAATGTGA
TCTGACCACAAAGAAAGTAGTAGACTACACAACAGCGAAGGAATTTGCTGATTC
CCTTGGAATTCCGTTTTTGGAAACCAGTGCTAAGAATGCAACGAATGTAGAACA
GTCTTTCATGACGATGGCAGCTGAGATTAAAAAGCGAATGGGTCCCGGAGCAAC
AGCTGGTGGTGCTGAGAAGTCCAATGTTAAAATTCAGAGCACTCCAGTCAAGCA
GTCAGGTGGAGGTTGCTGCTAA;
(Mouse Rab1a^{N124I} ORF Codon Seqeunce, SEQ ID NO: 8)
ATGGGGGACTACAAGGACGACGATGACAAGGGGGGTAGCGGTGGATCCAGCAT
GAATCCCGAATATGATTATTTATTCAAGTTACTTCTGATTGGCGATTCTGGGGTT
GGAAAGTCCTGCCTTCTCCTTAGGTTTGCAGATGATACGTATACGGAAAGCTACA
TCAGCACAATTGGTGTGGATTTCAAGATACGAACTATAGAGTTAGATGGGAAAA
CAATCAAGCTACAGATATGGGACACAGCAGGCCAGGAAAGATTTCGAACAATCA
CTTCCAGTTATTACAGAGGAGCCCATGGCATCATAGTTGTGTATGATGTGACAGA
TCAGGAGTCCTTCAATAACGTTAAACAGTGGCTGCAGGAGATAGATCGCTACGC
CAGTGAAAATGTCAACAAGTTGTTGGTAGGGATCAAATGTGACCTGACCACAAA
GAAAGTAGTAGACTACACAACAGCAAAGGAATTTGCAGATTCCCTTGGAATTCC
ATTTTTGGAAACCAGTGCTAAGAACGCAACGAATGTAGAACAGTCTTTCATGAC
GATGGCAGCTGAGATTAAAAAGCGAATGGGTCCTGGAGCTACAGCTGGTGGTGC
CGAGAAGTCCAATGTTAAAATCCAGAGCACTCCAGTCAAGCAGTCAGGTGGAGG
CTGCTGCTAA;
or
(Human Rab1a^{D47N} ORF Codon Sequence, SEQ ID NO: 17)
ATGTCCAGCATGAATCCCGAATATGATTATTTATTCAAGTTACTTCTGATTGGCGACT
CAGGGGTTGGAAAGTCTTGCCTTCTTCTTAGGTTTGCAGATGATACATATACAGAAA
GCTACATCAGCACAATTGGTGTGAACTTCAAAATAAGAACTATAGAGTTAGACGGG
AAAACAATCAAGCTTCAAATATGGGACACAGCAGGCCAGGAAAGATTTCGAACAAT
CACCTCCAGTTATTACAGAGGAGCCCATGGCATCATAGTTGTGTATGATGTGACAGA
TCAGGAGTCCTTCAATAATGTTAAACAGTGGCTGCAGGAAATAGATCGTTATGCCAG
TGAAAATGTCAACAAATTGTTGGTAGGGAACAAATGTGATCTGACCACAAAGAAAG
TAGTAGACTACACAACAGCGAAGGAATTTGCTGATTCCCTTGGAATTCCGTTTTTGG
AAACCAGTGCTAAGAATGCAACGAATGTAGAACAGTCTTTCATGACGATGGCAGCT
GAGATTAAAAAGCGAATGGGTCCCGGAGCAACAGCTGGTGGTGCTGAGAAGTCCAA
TGTTAAAATTCAGAGCACTCCAGTCAAGCAGTCAGGTGGAGGTTGCTGCTAA;

or a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity therewith and encoding a Rab1a^{GDP} preferentially binding GDP;
or a nucleic acid sequence equivalent to any of the above sequences due to codon redundancy.

In another embodiment of any of the above method or methods, the one or more expressible nucleic acids may comprise a nucleic acid sequence of:

(SEQ ID NO: 19, MG-008 ORF DNA Sequence with 5′ Luciferase Tag)
ATGGAAGACGCCAAAAACATAAAGAAAGGCCCGGCGCCATTCTATCCGCTAGAG
GATGGAACCGCTGGAGAGCAACTGCATAAGGCTATGAAGAGATACGCCCTGGTT
CCTGGAACAATTGCTTTTACAGATGCACATATCGAGGTGAACATCACGTACGCG
GAATACTTCGAAATGTCCGTTCGGTTGGCAGAAGCTATGAAACGATATGGGCTG
AATACAAATCACAGAATCGTCGTATGCAGTGAAAACTCTCTTCAATTCTTTATGC
CGGTGTTGGGCGCGTTATTTATCGGAGTTGCAGTTGCGCCCGCGAACGACATTTA
TAATGAACGTGAATTGCTCAACAGTATGAACATTTCGCAGCCTACCGTAGTGTTT
GTTTCCAAAAAGGGGTTGCAAAAAATTTTGAACGTGCAAAAAAAATTACCAATA
ATCCAGAAAATTATTATCATGGATTCTAAAACGGATTACCAGGGATTTCAGTCGA
TGTACACGTTCGTCACATCTCATCTACCTCCCGGTTTTAATGAATACGATTTTGTA
CCAGAGTCCTTTGATCGTGACAAAACAATTGCACTGATAATGAACTCCTCTGGAT
CTACTGGGTTACCTAAGGGTGTGGCCCTTCCGCATAGAACTGCCTGCGTCAGATT
CTCGCATGCCAGAGATCCTATTTTTGGCAATCAAATCATTCCGGATACTGCGATT
TTAAGTGTTGTTCCATTCCATCACGGTTTTGGAATGTTTACTACACTCGGATATTT
GATATGTGGATTTCGAGTCGTCTTAATGTATAGATTTGAAGAAGAGCTGTTTTTA
CGATCCCTTCAGGATTACAAAATTCAAAGTGCGTTGCTAGTACCAACCCTATTTT
CATTCTTCGCCAAAAGCACTCTGATTGACAAATACGATTTATCTAATTTACACGA
AATTGCTTCTGGGGGCGCACCTCTTTCGAAAGAAGTCGGGGAAGCGGTTGCAAA
ACGCTTCCATCTTCCAGGGATACGACAAGGATATGGGCTCACTGAGACTACATC
AGCTATTCTGATTACACCCGAGGGGGATGATAAACCGGGCGCGGTCGGTAAAGT
TGTTCCATTTTTTGAAGCGAAGGTTGTGGATCTGGATACCGGGAAAACGCTGGGC
GTTAATCAGAGAGGCGAATTATGTGTCAGAGGACCTATGATTATGTCCGGTTATG
TAAACAATCCGGAAGCGACCAACGCCTTGATTGACAAGGATGGATGGCTACATT
CTGGAGACATAGCTTACTGGGACGAAGACGAACACTTCTTCATAGTTGACCGCTT
GAAGTCTTTAATTAAATACAAAGGATACCAGGTGGCCCCCGCTGAATTGGAGTC
GATATTGTTACAACACCCCAACATCTTCGACGCGGGCGTGGCAGGTCTTCCCGAC
GATGACGCCGGTGAACTTCCCGCCGCCGTTGTTGTTTTGGAGCACGGAAAGACG
ATGACGGAAAAAGAGATCGTGGATTACGTCGCCAGTCAAGTAACAACCGCCAAA
AAGTTGCGCGGAGGAGTTGTGTTTGTGGACGAAGTACCGAAAGGTCTTACCGGA
AAACTCGACGCAAGAAAAATCAGAGAGATCCTCATAAAGGCCAAGAAGGGCGG
AAAGTCCAAATTGATGTCCAGCATGAATCCCGAATATGATTATTTATTCAAGTTA
CTTCTGATTGGCGACTCAGGGGTTGGAAAGAATTGCCTTCTTCTTAGGTTTGCAG
ATGATACATATACAGAAAGCTACATCAGCACAATTGGTGTGGATTTCAAAATAA
GAACTATAGAGTTAGACGGGAAAACAATCAAGCTTCAAATATGGGACACAGCAG
GCCAGGAAAGATTTCGAACAATCACCTCCAGTTATTACAGAGGAGCCCATGGCA
TCATAGTTGTGTATGATGTGACAGATCAGGAGTCCTTCAATAATGTTAAACAGTG
GCTGCAGGAAATAGATCGTTATGCCAGTGAAAATGTCAACAAATTGTTGGTAGG
GAACAAATGTGATCTGACCACAAAGAAAGTAGTAGACTACACAACAGCGAAGG
AATTTGCTGATTCCCTTGGAATTCCGTTTTTGGAAACCAGTGCTAAGAATGCAAC
GAATGTAGAACAGTCTTTCATGACGATGGCAGCTGAGATTAAAAAGCGAATGGG
TCCCGGAGCAACAGCTGGTGGTGCTGAGAAGTCCAATGTTAAAATTCAGAGCAC
TCCAGTCAAGCAGTCAGGTGGAGGTTGCTGCTAA;
or
(SEQ ID NO: 20, MG-008 ORF mRNA Sequence with 5′ Luciferase Tag)
AUGGAAGACGCCAAAAACAUAAAGAAAGGCCCGGCGCCAUUCUAUCCGCUAG
AGGAUGGAACCGCUGGAGAGCAACUGCAUAAGGCUAUGAAGAGAUACGCCCU
GGUUCCUGGAACAAUUGCUUUUACAGAUGCACAUAUCGAGGUGAACAUCACG
UACGCGGAAUACUUCGAAAUGUCCGUUCGGUUGGCAGAAGCUAUGAAACGAU
AUGGGCUGAAUACAAAUCACAGAAUCGUCGUAUGCAGUGAAAACUCUCUUCA
AUUCUUUAUGCCGGUGUUGGGCGCGUUAUUUAUCGGAGUUGCAGUUGCGCCC
GCGAACGACAUUUAUAAUGAACGUGAAUUGCUCAACAGUAUGAACAUUUCGC
AGCCUACCGUAGUGUUUGUUUCCAAAAAGGGGUUGCAAAAAAUUUUGAACGU
GCAAAAAAAAUUACCAAUAAUCCAGAAAAUUAUUAUCAUGGAUUCUAAAACG
GAUUACCAGGGAUUUCAGUCGAUGUACACGUUCGUCACAUCUCAUCUACCUCC
CGGUUUUAAUGAAUACGAUUUUGUACCAGAGUCCUUUGAUCGUGACAAAACA
AUUGCACUGAUAAUGAACUCCUCUGGAUCUACUGGGUUACCUAAGGGUGUGG
CCCUUCCGCAUAGAACUGCCUGCGUCAGAUUCUCGCAUGCCAGAGAUCCUAUU
UUUGGCAAUCAAAUCAUUCCGGAUACUGCGAUUUUAAGUGUUGUUCCAUUCC
AUCACGGUUUUGGAAUGUUUACUACACUCGGAUAUUUGAUAUGUGGAUUUCG
AGUCGUCUUAAUGUAUAGAUUUGAAGAAGAGCUGUUUUUACGAUCCCUUCAG
GAUUACAAAAUUCAAAGUGCGUUGCUAGUACCAACCCUAUUUUCAUUCUUCG
CCAAAAGCACUCUGAUUGACAAAUACGAUUUAUCUAAUUUACACGAAAUUGC
UUCUGGGGGCGCACCUCUUUCGAAAGAAGUCGGGGAAGCGGUUGCAAAACGC
UUCCAUCUUCCAGGGAUACGACAAGGAUAUGGGCUCACUGAGACUACAUCAG
CUAUUCUGAUUACACCCGAGGGGGAUGAUAAACCGGGCGCGGUCGGUAAAGU
UGUUCCAUUUUUUGAAGCGAAGGUUGUGGAUCUGGAUACCGGGAAAACGCUG
GGCGUUAAUCAGAGAGGCGAAUUAUGUGUCAGAGGACCUAUGAUUAUGUCCG
GUUAUGUAAACAAUCCGGAAGCGACCAACGCCUUGAUUGACAAGGAUGGAUG
GCUACAUUCUGGAGACAUAGCUUACUGGGACGAAGACGAACACUUCUUCAUA
GUUGACCGCUUGAAGUCUUUAAUUAAAUACAAAGGAUACCAGGUGGCCCCCG
CUGAAUUGGAGUCGAUAUUGUUACAACACCCCAACAUCUUCGACGCGGGCGU
GGCAGGUCUUCCCGACGAUGACGCCGGUGAACUUCCCGCCGCCGUUGUUGUUU
UGGAGCACGGAAAGACGAUGACGGAAAAAGAGAUCGUGGAUUACGUCGCCAG
UCAAGUAACAACCGCCAAAAAGUUGCGCGGAGGAGUUGUGUUUGUGGACGAA
GUACCGAAAGGUCUUACCGGAAAACUCGACGCAAGAAAAAUCAGAGAGAUCC
UCAUAAAGGCCAAGAAGGGCGGAAAGUCCAAAUUGAUGUCCAGCAUGAAUCC
CGAAUAUGAUUAUUUAUUCAAGUUACUUCUGAUUGGCGACUCAGGGGUUGGA
AAGAAUUGCCUUCUUCUUAGGUUUGCAGAUGAUACAUAUACAGAAAGCUACA
UCAGCACAAUUGGUGUGGAUUUCAAAAUAAGAACUAUAGAGUUAGACGGGAA
AACAAUCAAGCUUCAAAUAUGGGACACAGCAGGCCAGGAAAGAUUUCGAACA
AUCACCUCCAGUUAUUACAGAGGAGCCCAUGGCAUCAUAGUUGUGUAUGAUG
UGACAGAUCAGGAGUCCUUCAAUAAUGUUAAACAGUGGCUGCAGGAAAUAGA
UCGUUAUGCCAGUGAAAAUGUCAACAAAUUGUUGGUAGGGAACAAAUGUGAU
CUGACCACAAAGAAAGUAGUAGACUACACAACAGCGAAGGAAUUUGCUGAUU
CCCUUGGAAUUCCGUUUUUGGAAACCAGUGCUAAGAAUGCAACGAAUGUAGA
ACAGUCUUUCAUGACGAUGGCAGCUGAGAUUAAAAAGCGAAUGGGUCCCGGA
GCAACAGCUGGUGGUGCUGAGAAGUCCAAUGUUAAAAUUCAGAGCACUCCAG
UCAAGCAGUCAGGUGGAGGUUGCUGCUAA;

or a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity therewith and encoding a Rab1a^{GDP} preferentially binding GDP;
or a nucleic acid sequence equivalent to any of the above sequences due to codon redundancy.

In another embodiment, there is provided herein a use of a GDP-bound form of Rab1a (Rab1a^{GDP}), one or more expressible nucleic acids encoding Rab1a^{GDP}, or a combination thereof, for reducing symptoms of Parkinson's disease in a subject, or for preventing or treating Parkinson's disease, in a subject in need thereof.

In an embodiment, the symptoms of Parkinson's disease include at least one of the following: static tremor, postural imbalance, bradykinesia, muscle rigidity, punding, dyskinesias, hallucinations, impulse control disorder and sleep disorders.

In another embodiment, there is provided herein a use of a GDP-bound form of Rab1a (Rab1a^{GDP}), one or more expressible nucleic acids encoding Rab1a^{GDP}, or a combination thereof, for increasing cell viability of a dopaminergic neuron, or for preventing or treating dopaminergic neuronal death, in a subject in need thereof.

In another embodiment, there is provided herein a use of a GDP-bound form of Rab1a (Rab1a^{GDP}), one or more expressible nucleic acids encoding Rab1a^{GDP}, or a combination thereof, for increasing cell viability of dopaminergic neurons in vitro or in vivo.

In another embodiment, there is provided herein a use of a GDP-bound form of Rab1a (Rab1a^{GDP}), one or more expressible nucleic acids encoding Rab1a^{GDP}, or a combination thereof, in the manufacture of a medicament for reducing symptoms of Parkinson's disease, or for preventing or treating Parkinson's disease, in a subject in need thereof.

In an embodiment, the symptoms of Parkinson's disease include at least one of the following: static tremor, postural imbalance, bradykinesia, muscle rigidity, punding, dyskinesias, hallucinations, impulse control disorder and sleep disorders.

In another embodiment, there is provided herein a use of a GDP-bound form of Rab1a (Rab1a^{GDP}), one or more expressible nucleic acids encoding Rab1a^{GDP}, or a combination thereof, in the manufacture of a medicament for increasing cell viability of dopaminergic neurons, or for preventing or treating dopaminergic neuronal death, in a subject in need thereof.

In another embodiment, there is provided herein a use of a GDP-bound form of Rab1a (Rab1a^{GDP}), one or more expressible nucleic acids encoding Rab1a^{GDP}, or a combination thereof, in the manufacture of a medicament for increasing cell viability of dopaminergic neurons in vitro or in vivo.

In another embodiment of any of the above use or uses, the Rab1a^{GDP} may be or may comprise Rab1a^{S25N}, Rab1a^{N124I}, Rab1a^{D41N}, Rab1a^{D47N}, or another dominant negative (DN) GDP-bound form of Rab1a.

In still another embodiment of any of the above use or uses, the Rab1a^{GDP} may comprise the amino acid sequence:

(SEQ ID NO: 6; Human Rab1a^{S25N})
MSSMNPEYDYLFKLLLIGDSGVGKNCLLLRFADDTYTESYISTIGVDFK
IRTIELDGKTIKLQIWDTAGQERFRTITSSYYRGAHGIIVVYDVTDQES
FNNVKQWLQEIDRYASENVNKLLVGNKCDLTTKKVVDYTTAKEFADSLG
IPFLETSAKNATNVEQSFMTMAAEIKKRMGPGATAGGAEKSNVKIQSTP
VKQSGGGCC;
(SEQ ID NO: 9; Mouse Rab1^{N124I})
MGDYKDDDDKGGSGGSSMNPEYDYLFKLLLIGDSGVGKSCLLLRFADDT
YTESYISTIGVDFKIRTIELDGKTIKLQIWDTAGQERFRTITSSYYRGA
HGIIVVYDVTDQESENNVKQWLQEIDRYASENVNKLLVGIKCDLTTKKV
VDYTTAKEFADSLGIPFLETSAKNATNVEQSFMTMAAEIKKRMGPGATA
GGAEKSNVKIQSTPVKQSGGGCC;
the amino acid sequence of human Rab1a^{D41N},
or
(SEQ ID NO: 18; Human Rab1a^{D47N})
MSSMNPEYDYLFKLLLIGDSGVGKSCLLLRFADDTYTESYISTIGVNFK
IRTIELDGKTIKLQIWDTAGQERFRTITSSYYRGAHGIIVVYDVTDQES
FNNVKQWLQEIDRYASENVNKLLVGNKCDLTTKKVVDYTTAKEFADSLG
IPFLETSAKNATNVEQSFMTMAAEIKKRMGPGATAGGAEKSNVKIQSTP
VKQSGGGCC;

or a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity with any of these sequences and preferentially binding GDP.

In yet another embodiment of any of the above use or uses, the Rab1a^{GDP} may consist of the amino acid sequence:

(SEQ ID NO: 6; Human Rab1a^{S25N})
MSSMNPEYDYLFKLLLIGDSGVGKNCLLLRFADDTYTESYISTIGVDFK
IRTIELDGKTIKLQIWDTAGQERFRTITSSYYRGAHGIIVVYDVTDQES
FNNVKQWLQEIDRYASENVNKLLVGNKCDLTTKKVVDYTTAKEFADSLG
IPFLETSAKNATNVEQSFMTMAAEIKKRMGPGATAGGAEKSNVKIQSTP
VKQSGGGCC;
(SEQ ID NO: 9; Mouse Rab1^{N124I})
MGDYKDDDDKGGSGGSSMNPEYDYLFKLLLIGDSGVGKSCLLLRFADDT
YTESYISTIGVDFKIRTIELDGKTIKLQIWDTAGQERFRTITSSYYRGA
HGIIVVYDVTDQESFNNVKQWLQEIDRYASENVNKLLVGIKCDLTTKKV
VDYTTAKEFADSLGIPFLETSAKNATNVEQSFMTMAAEIKKRMGPGATA
GGAEKSNVKIQSTPVKQSGGGCC;
the amino acid sequence of human Rab1a^{D41N};
or
(SEQ ID NO: 18; Human Rab1a^{D47N})
MSSMNPEYDYLFKLLLIGDSGVGKSCLLLRFADDTYTESYISTIGVNFK
IRTIELDGKTIKLQIWDTAGQERFRTITSSYYRGAHGIIVVYDVTDQES
FNNVKQWLQEIDRYASENVNKLLVGNKCDLTTKKVVDYTTAKEFADSLG
IPFLETSAKNATNVEQSFMTMAAEIKKRMGPGATAGGAEKSNVKIQSTP
VKQSGGGCC;

or a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity with any of these sequences and preferentially binding GDP.

In another embodiment of any of the above use or uses, the Rab1a^{GDP} may comprise or consist of the amino acid sequence:

(SEQ ID NO: 6; Human Rab1a^{S25N})
MSSMNPEYDYLFKLLLIGDSGVGKNCLLLRFADDTYTESYISTIGVDFK
IRTIELDGKTIKLQIWDTAGQERFRTITSSYYRGAHGIIVVYDVTDQES
FNNVKQWLQEIDRYASENVNKLLVGNKCDLTTKKVVDYTTAKEFADSLG
IPFLETSAKNATNVEQSFMTMAAEIKKRMGPGATAGGAEKSNVKIQSTP
VKQSGGGCC;

or a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity therewith and preferentially binding GDP.

In still another embodiment of any of the above use or uses, the Rab1a^{GDP} may be in the form of a fusion protein, wherein the Rab1a^{GDP} is fused or otherwise directly or indirectly linked, optionally via a linker, with a signaling or targeting peptide, a fluorescent peptide or other marker or tracer, or another peptide or non-peptide moiety for targeted delivery, facilitating cell uptake, increasing stability or in vivo half-life, or improving another therapeutic, diagnostic, or in vivo property of the Rab1a^{GDP}.

In yet another embodiment of any of the above use or uses, the fusion protein may comprise the amino acid sequence:

(SEQ ID NO: 6; Human Rab1a^{S25N})
MSSMNPEYDYLFKLLLIGDSGVGKNCLLLRFADDTYTESYISTIGVDFK
IRTIELDGKTIKLQIWDTAGQERFRTITSSYYRGAHGIIVVYDVTDQES
FNNVKQWLQEIDRYASENVNKLLVGNKCDLTTKKVVDYTTAKEFADSLG
IPFLETSAKNATNVEQSFMTMAAEIKKRMGPGATAGGAEKSNVKIQSTP
VKQSGGGCC;

or a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity therewith and preferentially binding GDP.

In another embodiment of any of the above use or uses, the Rab1a^{GDP} may be in the form of a fusion protein, and may comprise the amino acid sequence:

(SEQ ID NO: 21)
MEDAKNIKKGPAPFYPLEDGTAGEQLHKAMKRYALVPGTIAFTDAHIEV
NITYAEYFEMSVRLAEAMKRYGLNTNHRIVVCSENSLQFFMPVLGALFI
GVAVAPANDIYNERELLNSMNISQPTVVFVSKKGLQKILNVQKKLPIIQ
KIIIMDSKTDYQGFQSMYTFVTSHLPPGFNEYDFVPESFDRDKTIALIM
NSSGSTGLPKGVALPHRTACVRFSHARDPIFGNQIIPDTAILSVVPFHH
GFGMFTTLGYLICGFRVVLMYRFEEELFLRSLQDYKIQSALLVPTLFSF
FAKSTLIDKYDLSNLHEIASGGAPLSKEVGEAVAKRFHLPGIRQGYGLT
ETTSAILITPEGDDKPGAVGKVVPFFEAKVVDLDTGKTLGVNQRGELCV
RGPMIMSGYVNNPEATNALIDKDGWLHSGDIAYWDEDEHFFIVDRLKSL
IKYKGYQVAPAELESILLQHPNIFDAGVAGLPDDDAGELPAAVVVLEHG
KTMTEKEIVDYVASQVTTAKKLRGGVVFVDEVPKGLTGKLDARKIREIL
IKAKKGGKSKLMSSMNPEYDYLFKLLLIGDSGVGKNCLLLRFADDTYTE
SYISTIGVDFKIRTIELDGKTIKLQIWDTAGQERFRTITSSYYRGAHGI
IVVYDVTDQESFNNVKQWLQEIDRYASENVNKLLVGNKCDLTTKKVVDY
TTAKEFADSLGIPFLETSAKNATNVEQSFMTMAAEIKKRMGPGATAGGA
EKSNVKIQSTPVKQSGGGCC;

or a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity therewith and preferentially binding GDP.

In still another embodiment of any of the above use or uses, the one or more expressible nucleic acids may encode one or more Rab1a^{GDP}s as defined herein.

In yet another embodiment of any of the above use or uses, the one or more expressible nucleic acids may be DNA-based, or RNA-based.

In another embodiment of any of the above use or uses, the one or more expressible nucleic acids may transiently express the Rab1a^{GDP} in the dopaminergic neuron, or wherein the one or more expressible nucleic acids may integrate in the cell genome and express the Rab1a^{GDP} in the dopaminergic neuron.

In another embodiment of any of the above use or uses, the one or more expressible nucleic acids may comprise one or more expression vectors, plasmids, or mRNAs encoding and capable of expressing the Rab1a^{GDP} inside the cell.

In another embodiment of any of the above use or uses, the one or more expressible nucleic acids may comprise a nucleic acid sequence of:

(Human Rab1a^{S25N} ORF Codon Sequence, SEQ ID NO: 5)
ATGTCCAGCATGAATCCCGAATATGATTATTTATTCAAGTTACTTCTGATTGGCG
ACTCAGGGGTTGGAAAGAATTGCCTTCTTCTTAGGTTTGCAGATGATACATATAC
AGAAAGCTACATCAGCACAATTGGTGTGGATTTCAAAATAAGAACTATAGAGTT
AGACGGGAAAACAATCAAGCTTCAAATATGGGACACAGCAGGCCAGGAAAGAT
TTCGAACAATCACCTCCAGTTATTACAGAGGAGCCCATGGCATCATAGTTGTGTA
TGATGTGACAGATCAGGAGTCCTTCAATAATGTTAAACAGTGGCTGCAGGAAAT
AGATCGTTATGCCAGTGAAAATGTCAACAAATTGTTGGTAGGGAACAAATGTGA
TCTGACCACAAAGAAAGTAGTAGACTACACAACAGCGAAGGAATTTGCTGATTC
CCTTGGAATTCCGTTTTTGGAAACCAGTGCTAAGAATGCAACGAATGTAGAACA
GTCTTTCATGACGATGGCAGCTGAGATTAAAAAGCGAATGGGTCCCGGAGCAAC
AGCTGGTGGTGCTGAGAAGTCCAATGTTAAAATTCAGAGCACTCCAGTCAAGCA
GTCAGGTGGAGGTTGCTGCTAA;
(Mouse Rabla^{N124I} ORF Codon Seqeunce, SEQ ID NO: 8)
ATGGGGGACTACAAGGACGACGATGACAAGGGGGGTAGCGGTGGATCCAGCAT
GAATCCCGAATATGATTATTTATTCAAGTTACTTCTGATTGGCGATTCTGGGGTT
GGAAAGTCCTGCCTTCTCCTTAGGTTTGCAGATGATACGTATACGGAAAGCTACA
TCAGCACAATTGGTGTGGATTTCAAGATACGAACTATAGAGTTAGATGGGAAAA
CAATCAAGCTACAGATATGGGACACAGCAGGCCAGGAAAGATTTCGAACAATCA
CTTCCAGTTATTACAGAGGAGCCCATGGCATCATAGTTGTGTATGATGTGACAGA
TCAGGAGTCCTTCAATAACGTTAAACAGTGGCTGCAGGAGATAGATCGCTACGC
CAGTGAAAATGTCAACAAGTTGTTGGTAGGGATCAAATGTGACCTGACCACAAA
GAAAGTAGTAGACTACACAACAGCAAAGGAATTTGCAGATTCCCTTGGAATTCC
ATTTTTGGAAACCAGTGCTAAGAACGCAACGAATGTAGAACAGTCTTTCATGAC
GATGGCAGCTGAGATTAAAAAGCGAATGGGTCCTGGAGCTACAGCTGGTGGTGC
CGAGAAGTCCAATGTTAAAATCCAGAGCACTCCAGTCAAGCAGTCAGGTGGAGG
CTGCTGCTAA;
or
(Human Rab1a^{D47N} ORF Codon Sequence, SEQ ID NO: 17)
ATGTCCAGCATGAATCCCGAATATGATTATTTATTCAAGTTACTTCTGATTGGCGACT
CAGGGGTTGGAAAGTCTTGCCTTCTTCTTAGGTTTGCAGATGATACATATACAGAAA
GCTACATCAGCACAATTGGTGTGAACTTCAAAATAAGAACTATAGAGTTAGACGGG
AAAACAATCAAGCTTCAAATATGGGACACAGCAGGCCAGGAAAGATTTCGAACAAT
CACCTCCAGTTATTACAGAGGAGCCCATGGCATCATAGTTGTGTATGATGTGACAGA
TCAGGAGTCCTTCAATAATGTTAAACAGTGGCTGCAGGAAATAGATCGTTATGCCAG
TGAAAATGTCAACAAATTGTTGGTAGGGAACAAATGTGATCTGACCACAAAGAAAG
TAGTAGACTACACAACAGCGAAGGAATTTGCTGATTCCCTTGGAATTCCGTTTTTGG
AAACCAGTGCTAAGAATGCAACGAATGTAGAACAGTCTTTCATGACGATGGCAGCT
GAGATTAAAAAGCGAATGGGTCCCGGAGCAACAGCTGGTGGTGCTGAGAAGTCCAA
TGTTAAAATTCAGAGCACTCCAGTCAAGCAGTCAGGTGGAGGTTGCTGCTAA;

or a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity therewith and encoding a Rab1a^{GDP} preferentially binding GDP;
or a nucleic acid sequence equivalent to any of the above sequences due to codon redundancy.

In still another embodiment of any of the above use or uses, the one or more expressible nucleic acids may comprise a nucleic acid sequence of:

(SEQ ID NO: 19, MG-008 ORF DNA Sequence with 5′ Luciferase Tag)
ATGGAAGACGCCAAAAACATAAAGAAAGGCCCGGCGCCATTCTATCCGCTAGAG
GATGGAACCGCTGGAGAGCAACTGCATAAGGCTATGAAGAGATACGCCCTGGTT
CCTGGAACAATTGCTTTTACAGATGCACATATCGAGGTGAACATCACGTACGCG
GAATACTTCGAAATGTCCGTTCGGTTGGCAGAAGCTATGAAACGATATGGGCTG
AATACAAATCACAGAATCGTCGTATGCAGTGAAAACTCTCTTCAATTCTTTATGC
CGGTGTTGGGCGCGTTATTTATCGGAGTTGCAGTTGCGCCCGCGAACGACATTTA
TAATGAACGTGAATTGCTCAACAGTATGAACATTTCGCAGCCTACCGTAGTGTTT
GTTTCCAAAAAGGGGTTGCAAAAAATTTTGAACGTGCAAAAAAAATTACCAATA
ATCCAGAAAATTATTATCATGGATTCTAAAACGGATTACCAGGGATTTCAGTCGA
TGTACACGTTCGTCACATCTCATCTACCTCCCGGTTTTAATGAATACGATTTTGTA
CCAGAGTCCTTTGATCGTGACAAAACAATTGCACTGATAATGAACTCCTCTGGAT
CTACTGGGTTACCTAAGGGTGTGGCCCTTCCGCATAGAACTGCCTGCGTCAGATT
CTCGCATGCCAGAGATCCTATTTTTGGCAATCAAATCATTCCGGATACTGCGATT
TTAAGTGTTGTTCCATTCCATCACGGTTTTGGAATGTTTACTACACTCGGATATTT
GATATGTGGATTTCGAGTCGTCTTAATGTATAGATTTGAAGAAGAGCTGTTTTTA
CGATCCCTTCAGGATTACAAAATTCAAAGTGCGTTGCTAGTACCAACCCTATTTT
CATTCTTCGCCAAAAGCACTCTGATTGACAAATACGATTTATCTAATTTACACGA
AATTGCTTCTGGGGGCGCACCTCTTTCGAAAGAAGTCGGGGAAGCGGTTGCAAA
ACGCTTCCATCTTCCAGGGATACGACAAGGATATGGGCTCACTGAGACTACATC
AGCTATTCTGATTACACCCGAGGGGGATGATAAACCGGGCGCGGTCGGTAAAGT
TGTTCCATTTTTTGAAGCGAAGGTTGTGGATCTGGATACCGGGAAAACGCTGGGC
GTTAATCAGAGAGGCGAATTATGTGTCAGAGGACCTATGATTATGTCCGGTTATG
TAAACAATCCGGAAGCGACCAACGCCTTGATTGACAAGGATGGATGGCTACATT
CTGGAGACATAGCTTACTGGGACGAAGACGAACACTTCTTCATAGTTGACCGCTT
GAAGTCTTTAATTAAATACAAAGGATACCAGGTGGCCCCCGCTGAATTGGAGTC
GATATTGTTACAACACCCCAACATCTTCGACGCGGGCGTGGCAGGTCTTCCCGAC
GATGACGCCGGTGAACTTCCCGCCGCCGTTGTTGTTTTGGAGCACGGAAAGACG
ATGACGGAAAAAGAGATCGTGGATTACGTCGCCAGTCAAGTAACAACCGCCAAA
AAGTTGCGCGGAGGAGTTGTGTTTGTGGACGAAGTACCGAAAGGTCTTACCGGA
AAACTCGACGCAAGAAAAATCAGAGAGATCCTCATAAAGGCCAAGAAGGGCGG
AAAGTCCAAATTGATGTCCAGCATGAATCCCGAATATGATTATTTATTCAAGTTA
CTTCTGATTGGCGACTCAGGGGTTGGAAAGAATTGCCTTCTTCTTAGGTTTGCAG
ATGATACATATACAGAAAGCTACATCAGCACAATTGGTGTGGATTTCAAAATAA
GAACTATAGAGTTAGACGGGAAAACAATCAAGCTTCAAATATGGGACACAGCAG
GCCAGGAAAGATTTCGAACAATCACCTCCAGTTATTACAGAGGAGCCCATGGCA
TCATAGTTGTGTATGATGTGACAGATCAGGAGTCCTTCAATAATGTTAAACAGTG
GCTGCAGGAAATAGATCGTTATGCCAGTGAAAATGTCAACAAATTGTTGGTAGG
GAACAAATGTGATCTGACCACAAAGAAAGTAGTAGACTACACAACAGCGAAGG
AATTTGCTGATTCCCTTGGAATTCCGTTTTTGGAAACCAGTGCTAAGAATGCAAC
GAATGTAGAACAGTCTTTCATGACGATGGCAGCTGAGATTAAAAAGCGAATGGG
TCCCGGAGCAACAGCTGGTGGTGCTGAGAAGTCCAATGTTAAAATTCAGAGCAC
TCCAGTCAAGCAGTCAGGTGGAGGTTGCTGCTAA;
or
(SEQ ID NO: 20, MG-008 ORF mRNA Sequence with 5′ Luciferase Tag)
AUGGAAGACGCCAAAAACAUAAAGAAAGGCCCGGCGCCAUUCUAUCCGCUAG
AGGAUGGAACCGCUGGAGAGCAACUGCAUAAGGCUAUGAAGAGAUACGCCCU
GGUUCCUGGAACAAUUGCUUUUACAGAUGCACAUAUCGAGGUGAACAUCACG
UACGCGGAAUACUUCGAAAUGUCCGUUCGGUUGGCAGAAGCUAUGAAACGAU
AUGGGCUGAAUACAAAUCACAGAAUCGUCGUAUGCAGUGAAAACUCUCUUCA
AUUCUUUAUGCCGGUGUUGGGCGCGUUAUUUAUCGGAGUUGCAGUUGCGCCC
GCGAACGACAUUUAUAAUGAACGUGAAUUGCUCAACAGUAUGAACAUUUCGC
AGCCUACCGUAGUGUUUGUUUCCAAAAAGGGGUUGCAAAAAAUUUUGAACGU
GCAAAAAAAAUUACCAAUAAUCCAGAAAAUUAUUAUCAUGGAUUCUAAAACG
GAUUACCAGGGAUUUCAGUCGAUGUACACGUUCGUCACAUCUCAUCUACCUCC
CGGUUUUAAUGAAUACGAUUUUGUACCAGAGUCCUUUGAUCGUGACAAAACA
AUUGCACUGAUAAUGAACUCCUCUGGAUCUACUGGGUUACCUAAGGGUGUGG
CCCUUCCGCAUAGAACUGCCUGCGUCAGAUUCUCGCAUGCCAGAGAUCCUAUU
UUUGGCAAUCAAAUCAUUCCGGAUACUGCGAUUUUAAGUGUUGUUCCAUUCC
AUCACGGUUUUGGAAUGUUUACUACACUCGGAUAUUUGAUAUGUGGAUUUCG
AGUCGUCUUAAUGUAUAGAUUUGAAGAAGAGCUGUUUUUACGAUCCCUUCAG
GAUUACAAAAUUCAAAGUGCGUUGCUAGUACCAACCCUAUUUUCAUUCUUCG
CCAAAAGCACUCUGAUUGACAAAUACGAUUUAUCUAAUUUACACGAAAUUGC
UUCUGGGGGCGCACCUCUUUCGAAAGAAGUCGGGGAAGCGGUUGCAAAACGC
UUCCAUCUUCCAGGGAUACGACAAGGAUAUGGGCUCACUGAGACUACAUCAG
CUAUUCUGAUUACACCCGAGGGGGAUGAUAAACCGGGCGCGGUCGGUAAAGU
UGUUCCAUUUUUUGAAGCGAAGGUUGUGGAUCUGGAUACCGGGAAAACGCUG
GGCGUUAAUCAGAGAGGCGAAUUAUGUGUCAGAGGACCUAUGAUUAUGUCCG
GUUAUGUAAACAAUCCGGAAGCGACCAACGCCUUGAUUGACAAGGAUGGAUG
GCUACAUUCUGGAGACAUAGCUUACUGGGACGAAGACGAACACUUCUUCAUA
GUUGACCGCUUGAAGUCUUUAAUUAAAUACAAAGGAUACCAGGUGGCCCCCG
CUGAAUUGGAGUCGAUAUUGUUACAACACCCCAACAUCUUCGACGCGGGCGU
GGCAGGUCUUCCCGACGAUGACGCCGGUGAACUUCCCGCCGCCGUUGUUGUUU
UGGAGCACGGAAAGACGAUGACGGAAAAAGAGAUCGUGGAUUACGUCGCCAG
UCAAGUAACAACCGCCAAAAAGUUGCGCGGAGGAGUUGUGUUUGUGGACGAA
GUACCGAAAGGUCUUACCGGAAAACUCGACGCAAGAAAAAUCAGAGAGAUCC
UCAUAAAGGCCAAGAAGGGCGGAAAGUCCAAAUUGAUGUCCAGCAUGAAUCC
CGAAUAUGAUUAUUUAUUCAAGUUACUUCUGAUUGGCGACUCAGGGGUUGGA
AAGAAUUGCCUUCUUCUUAGGUUUGCAGAUGAUACAUAUACAGAAAGCUACA
UCAGCACAAUUGGUGUGGAUUUCAAAAUAAGAACUAUAGAGUUAGACGGGAA
AACAAUCAAGCUUCAAAUAUGGGACACAGCAGGCCAGGAAAGAUUUCGAACA
AUCACCUCCAGUUAUUACAGAGGAGCCCAUGGCAUCAUAGUUGUGUAUGAUG
UGACAGAUCAGGAGUCCUUCAAUAAUGUUAAACAGUGGCUGCAGGAAAUAGA
UCGUUAUGCCAGUGAAAAUGUCAACAAAUUGUUGGUAGGGAACAAAUGUGAU
CUGACCACAAAGAAAGUAGUAGACUACACAACAGCGAAGGAAUUUGCUGAUU
CCCUUGGAAUUCCGUUUUUGGAAACCAGUGCUAAGAAUGCAACGAAUGUAGA
ACAGUCUUUCAUGACGAUGGCAGCUGAGAUUAAAAAGCGAAUGGGUCCCGGA
GCAACAGCUGGUGGUGCUGAGAAGUCCAAUGUUAAAAUUCAGAGCACUCCAG
UCAAGCAGUCAGGUGGAGGUUGCUGCUAA;

or a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity therewith and encoding a Rab1a^{GDP} preferentially binding GDP;
or a nucleic acid sequence equivalent to any of the above sequences due to codon redundancy.

In another embodiment, there is provided herein a polypeptide comprising the amino acid sequence:

(SEQ ID NO: 6; Human Rab1a^{S25N})
MSSMNPEYDYLFKLLLIGDSGVGKNCLLLRFADDTYTESYISTIGVDFKI
RTIELDGKTIKLQIWDTAGQERFRTITSSYYRGAHGIIVVYDVTDQESFN
NVKQWLQEIDRYASENVNKLLVGNKCDLTTKKVVDYTTAKEFADSLGIPF
LETSAKNATNVEQSFMTMAAEIKKRMGPGATAGGAEKSNVKIQSTPVKQS
GGGCC;
(SEQ ID NO: 9; Mouse Rab1^{N124I})
MGDYKDDDDKGGSGGSSMNPEYDYLFKLLLIGDSGVGKSCLLLRFADDTY
TESYISTIGVDFKIRTIELDGKTIKLQIWDTAGQERFRTITSSYYRGAHG
IIVVYDVTDQESFNNVKQWLQEIDRYASENVNKLLVGIKCDLTTKKVVDY
TTAKEFADSLGIPFLETSAKNATNVEQSFMTMAAEIKKRMGPGATAGGAE
KSNVKIQSTPVKQSGGGCC;

the amino acid sequence of human Rab1a^{D41N}; or

(SEQ ID NO: 18; Human Rab1a^{D47N})
MSSMNPEYDYLFKLLLIGDSGVGKSCLLLRFADDTYTESYISTIGVNFKI
RTIELDGKTIKLQIWDTAGQERFRTITSSYYRGAHGIIVVYDVTDQESFN
NVKQWLQEIDRYASENVNKLLVGNKCDLTTKKVVDYTTAKEFADSLGIPF
LETSAKNATNVEQSFMTMAAEIKKRMGPGATAGGAEKSNVKIQSTPVKQS
GGGCC;

or a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity with any of these sequences and preferentially binding GDP;
for use in increasing cell viability of dopaminergic neurons, or for preventing or treating Parkinson's disease, in a subject in need thereof; or for use in increasing cell viability of dopaminergic neurons in vitro or in vivo.

In another embodiment, there is provided herein a pharmaceutical composition comprising
a GDP-bound form of Rab1a (Rab1a^{GDP}), one or more expressible nucleic acids encoding Rab1a^{GDP}, or a combination thereof; and another anti-Parkinson agent.

In yet another embodiment, there is provided herein a kit comprising any one or more of:
a GDP-bound form of Rab1a (Rab1a^{GDP});
one or more expressible nucleic acids encoding Rab1a^{GDP};
an anti-Parkinson agent;
instructions for performing any of the method or methods as described herein; or
any combinations thereof.

In still another embodiment of any of the above methods, uses, or polypeptides for use, the symptoms may be static tremor, postural imbalance, bradykinesia, muscle rigidity, punding, dyskinesias, hallucinations, impulse control disorder and sleep disorders.

### BRIEF DESCRIPTION OF DRAWINGS

These and other features will become further understood having regard to the following drawings in which male Sprague-Dawley rats with symptoms of Parkinson's disease were treated with mRNA-encoded Rab1a^{GDP}:

FIG. 1 shows that administration of MG-008 in hemiparkinsonian rat models generated by intracranial injection of 6-hydroxydopamine (also known as oxidopamine or 2,4,5-trihydrxyphenethylamine; 6-OHDA) resulted in increased latency to fall (in seconds) (A, C) and increased speed at fall (B, D) three weeks post-MG-008 administration (A-B) and five weeks post-MG-008 administration (C-D), as described in Example 1;

FIG. 2 shows that administration of MG-008 in hemiparkinsonian rat models generated by intracranial injection of 6-OHDA resulted in decreased time (in seconds) required for the rats to cross the beam four weeks post-MG008 administration, as described in Example 1;

FIG. 3 shows that administration of MG-008 in hemiparkinsonian rat models generated by intracranial injection of 6-OHDA resulted in reduced turning rates expressed as mean 360°/hour of rats in a rotometer apparatus four weeks post-MG008 administration, as described in Example 1;

FIG. 4 shows that administration of MG-008 in hemiparkinsonian rat models generated by intracranial injection of 6-OHDA resulted in restored dopaminergic neurons in the substantia nigra pars compacta (SNpc) in hemiparkinsonian rats (B) compared to control (A) and restored dopaminergic neurons in the substantia nigra pars reticulata (SNr) in hemiparkinsonian rats compared to control (A), as described in Example 1;

FIG. 5 shows certain amino acid sequences and nucleic acid sequences as described herein.

FIG. 6 shows an example of the experimental schedule for F11 administration in hemiparkinsonian rat models, which were generated by intracranial injection of 6-OHDA.

FIG. 7 shows another example of an experimental schedule for F11 administration in hemiparkinsonian rat models, which were generated by intracranial injection of 6-OHDA.

FIG. 8 shows that average body weight (A) and average body weight change (B) did not change significantly between F11-treated rats and untreated (Sham) and 6-OHDA-treated rats.

FIG. 9 shows that the latency to fall in the rotorod test improved significantly in hemiparkinsonian rats (A) 2, (B) 4, (C) 8, and (D) 12 weeks after the first F11 intravenous injection.

FIG. 10 shows that the beam walking time decreased (A) 2, (B) 4, (C) 8, and (D) 12 weeks after the first F11 intravenous injection in hemiparkinsonian rat models.

FIG. 11 shows that a reduced turning rate occurred at (A) 2, (B) 4, (C) 8, and (D) 12 weeks after the first F11 intravenous injection in hemiparkinsonian rat models.

FIG. 12 shows increased tyrosine hydroxylase expression, determined by immunohistochemical staining, in the substantia nigra of hemiparkinsonian rat models following F11 administration.

FIG. 13 shows representative images of tyrosine hydroxylase expression in the substantia nigra of hemiparkinsonian rat models following F11 administration.

### DETAILED DESCRIPTION

Described herein are compounds, compositions, uses, and methods for increasing cell viability of a dopaminergic neuron in vitro or in vivo, and/or for preventing or treating symptoms of Parkinson's disease in a dopaminergic neuron or subject in need thereof. It will be appreciated that embodiments and examples are provided for illustrative purposes intended for those skilled in the art and are not meant to be limiting in any way.

As the second-most common neurodegenerative disorder affecting 2-3% of the population ≥65 years of age, Parkinson's disease (PD) results primarily from the death of dopaminergic neurons in the substantia nigra of the midbrain. Although the pathological mechanisms are poorly understood, both genetic and environmental factors are considered to be involved in the pathogenesis of PD (Gan-Or et al., 2015). The main obstacle to developing neuroprotective therapies is a limited understanding of the key molecular events that provoke neurodegeneration and more specifically, the death of dopaminergic neurons.

A missense mutation in α-synuclein (A53T), encoded by the SNCA/PARK1 gene, was the first PD-causing mutation identified in a familial form of PD (Polymeropoulos et al., 1997). Subsequently, a series of missense point mutations were identified in the SNCA gene which confirmed a centrol role for α-synuclein in the development of PD. Indeed, mutations in α-synuclein are toxic to dopaminergic neurons as they alter a series of intracellular signal programs. The A53T mutation was shown to suppress autophagy in the brain of transgenic mice at early stage which lead to α-synucleinopathies (Pupyshev et al., 2017), characterized abnormal accumulation of aggregates of alpha-synuclein protein in neurons, nerve fibres or glial cells. These α-synuclein aggregates also causes mitochondrial dysfunction, endoplasmic reticulum stress-mediated cell death pathways (Smith et al., 2005), and by binding to TrkB receptors, inhibit BDNF/TrkB signaling, resulting in dopaminergic neuronal death (Zharikov et al., 2015). Other mutations in α-synuclein was shown to impair autophagy such as the E46K mutation which inactivated the JNK1-Bcl-2 pathway or the mutation A30P mutation which was rescued by HMGB1 or Beclin upregulation in PC12 cells (Wang et al., 2016).

Another PD-causing mutation was found in the LRRK2/PARK8 gene and represents the highest risk of familial PD, causing autosomal dominant PD (Zimprich et al., 2004). LRRK2 mutations are linked with “classical” late-onset PD and accounts for 4% of hereditary PD (Ferreira and Massano, 2017). Leucine-rich repeat kinase 2 (LRRK2) is a multi-domain protein, with both Rab GTPase and kinase activities (Steger et al., 2017). LRRK2 G2019S mutation enhances the kinase activity and results in the impairment of synaptic vesicle trafficking selectively in ventral midbrain neurons, including dopaminergic neurons (Pan et al., 2017). This particular LRRK2 G2019S mutation was shown to, enhance α-synuclein accumulation (Volpicelli-Daley et al., 2016), induce ASK1-mediated apoptosis (Yoon et al., 2017), increased mitochondrial DLP1 which caused mitochondrial fragmentation and dysfunction (Wang et al., 2015), and dysregulated autophagy (Su et al., 2015).

Mutations in in PTEN-induced putative kinase (PINK) 1, a mitochondrial protein kinase, was also shown to be a critical protein linked to mitochondrial dysfunction and the pathogenesis of PD. Mutation of PINK1 show an accumulation of dysfunctional mitochondria which activates the mitochondrial unfolded protein response. Parkin, encoded by PARK2, is a ubiquitin E3 ligase, which is activated by autophosphorylated PINK1 which accumulates at the membrane of dysfunctional mitochondria to activate the E3 ubiquitin ligase activity of Parkin, thereby recruiting them to damaged mitochondria to promote their mitophagic degradation (Narendra et al., 2009). Mutations of Parkin such as R42P, R46P, K211N, C212Y, C253Y, C289G, and C441R, impairs recruitment to depolarized mitochondria and inhibit mitophagy (Narendra et al., 2010).

Mutations of the Vacuolar protein sorting 35 (VPS35) gene accounts for 1% of familial PD as the VPS35 protein regulates the process of transmembrane protein sorting between endosomes and the Golgi (Bonifacino and Hurley, 2008). VPS35-deficient dopaminergic neurons exhibit impaired endosome-to-Golgi retrieval of Lamp2a, which contribute to reduced α-synuclein degradation through chaperone-mediated autophagy (Tang et al., 2015).

A common theme in the pathogenesis of PD is the accumulation of misfolded proteins and defects in the mechanisms to remove them in order to prevent cytotoxicity of dopaminergic neurons. Indeed, autophagy is often affected early in PD and plays a critical role in the clearance of accumulated misfolded proteins and degradation of damaged organelles, which is widely considered to be cyto-protective. Interaction between PINK-1 and α-synuclein in the cytoplasm enhances the degradation of cytotoxic α-synuclein via activation of autophagy (Liu et al., 2017). Endoplasmic reticulum stressors activate recruit MKK4 to lysosomes and triggers chaperone-mediated autophagy to provide a pro-survival mechanism (Li et al., 2017). In contrast, autophagy is often defective or inhibited in PD and has been suggested to be a causative mechanism in the development of PD (Cai et al., 2015). Furthermore, inhibition of autophagy increases the release and transfer of α-synuclein from cell to cell via extracellular vesicles, which promotes the progression of synucleinopathies. Moreover, α-synuclein mutations, such as A53T and A30P, allows α-synuclein to bind to receptors on lysosomal membranes and inhibit chaperone-mediated autophagy, resulting in accumulation of abnormal α-synuclein in the Lewy bodies and subsequent parkinsonism. In addition, inhibition of chaperone-mediated autophagy results in progressive loss of dopaminergic neurons in the SNc, severe decrease of dopamine levels in the striatum, and motor deficits (Xilouri et al., 2016). Thus, promoting autophagy may be a reasonable strategy to target early onset of PD.

Autophagy is a major intracellular degradation system that derives its degradative abilities from the lysosome. It is a homeostatic and evolutionarily conserved mechanism of self-digestion by which the cells degrade and recycle long-lived proteins and excess or damaged organelles. Autophagy is a highly regulated process by which misfolded proteins and organelles reach lysosomes for their degradation (Kim K H, Lee M S. Autophagy—a key player in cellular and body metabolism. Nat Rev Endocrinol. 2014 June; 10 (6): 322-37). There are 3 different types of autophagy, namely macroautophagy (commonly known as autophagy), chaperon mediated autophagy, and microautophagy. Lysosomal degradation of substrates in chaperon mediate autophagy and microautophagy occurs directly within the lysosomes (Parzych K R, Klionsky D J. An overview of autophagy: morphology, mechanism, and regulation. Antioxid Redox Signal. 2014 Jan. 20; 20 (3): 460-73).

Cell death may include or involve different factors such as change in morphology of the cells or their functions. There are three major types of cell death: apoptosis (programmed cell death type 1), autophagy (programmed cell death type 2), and necroptosis (programmed cell death type 3) (Fuchs Y, Steller H. Programmed cell death in animal development and disease. Cell. 2011 Nov. 11; 147 (4): 742-58). In general, autophagy functions in cell-protective events. However, it may also be utilized as a cell-suicide mechanism, which is known as “autophagic cell death”. The process of autophagic cell death thus differs from apoptotic or necroptotic programmed cell death (Kroemer G, Levine B. Autophagic cell death: the story of a misnomer. Nat Rev Mol Cell Biol. 2008 December; 9 (12): 1004-10).

In this regard, the present inventors recognized that in addition to apoptosis (programmed cell death) and necroptosis, autophagic lysosome-mediated cell death, particularly the process of microautophagy, may be used for developing an effective strategy for treating Parkinson's disease.

Stimulation of microautophagy (which in certain embodiments may involve piecemeal engulfment of target membrane(s)) by promoting lysosome movement toward the target membranes) may be particularly desirable for treatment of a variety of diseases and/or disorders and/or conditions. Specific lysosome positioning inside the cells may be associated with different types of lysosome activity. In addition, lysosome positioning may correlate with the activity of mTOR and may regulate autophagic flux. During macroautophagy, mTORC1 may be inactive and lysosomes may be accumulated in the perinuclear region of the cells, which may perform macroautophagy by stimulation of fusion of encased target membrane with lysosomes. In contrast, in the process of microautophagy, piecemeal engulfment of target membrane(s) by lysosomes may be happening through lysosome movement and direct interaction with target membrane(s) without need of formation of autophagosome(s). Lysosomes may move in a bidirectional manner along the microtubules, and such a lysosomal motility is governed by different sets of motor proteins that are recruited through different mechanisms.

In certain embodiments, microautophagy may involve direct engulfment of cytoplasmic cargo at a boundary membrane by autophagic tubes, which may mediate both invagination and vesicle scission into the lumen of lysosomes (see Li, W.-W., Li, J. & Bao, J.-K. Microautophagy: lesser-known self-eating. Cell. Mol. Life Sci. 69, 1125-1136 (2011)). Direct lysosomal degradation of target substrates may occur for DNA, for example (referred to as a piecemeal autophagy—see Fujiwara, Y. et al. Direct uptake and degradation of DNA by lysosomes. —PubMed—NCBI. Autophagy 9, 1167-1171 (2014)). In addition, lysosomes may be able to move toward the different organelles and/or membranes substrates (e.g. plasma membrane, mitochondria) and may interact with them directly by recruitment of motor proteins and SNAREs proteins (see Andrews, N. W. Lysosomes and the plasma membrane. J. Cell Biol. 158, 389-394 (2002); Hofmann, I. & Munro, S. An N-terminally acetylated Arf-like GTPase is localised to lysosomes and affects their motility. J. Cell. Sci. 119, 1494-1503 (2006); Fraldi, A. et al. Lysosomal fusion and SNARE function are impaired by cholesterol accumulation in lysosomal storage disorders. The EMBO Journal 29, 3607-3620 (2010); and Pankiv, S. et al. FYCO1 is a Rab7 effector that binds to LC3 and PI3P to mediate microtubule plus end-directed vesicle transport. J. Cell Biol. 188, 253-269 (2010)). Movement of lysosomes toward the cell periphery and their positioning within the cells may be associated with signaling (see R. & Bonifacino, J. S. Lysosome Positioning Influences mTORC2 and AKT Signaling. Molecular Cell 75, 26-38.e3 (2019)). In this regard, it is contemplated that mTORC1, mTORC2 and AKT activation may be important for lysosomal peripheral distribution (see Poüs, C. & Codogno, P. Lysosome positioning coordinates mTORC1 activity and autophagy. Nature Cell Biology 13, 342-344 (2011); and Cabukusta, B. & Neefjes, J. Mechanisms of lysosomal positioning and movement. Traffic 19, 761-769 (2018)).

In certain embodiments, microautophagy and/or piecemeal degradation of target substrates and/or membranes may involve movement of lysosomes toward the peripheral and target membrane in cytosol (e.g. direct interaction with target membrane) (see Pu, J., Guardia, C. M., Keren-Kaplan, T. & Bonifacino, J. S. Mechanisms and functions of lysosome positioning. J. Cell. Sci. 129, 4329-4339 (2016); and Katherine R Parzych, D. J. K. An Overview of Autophagy: Morphology, Mechanism, and Regulation. Antioxid. Redox Signal. 20, 460-473 (2014)). In certain embodiments, movement of lysosomes toward the cell periphery (from perinuclear region of the cell) and interaction with target membranes/substrates (e.g. glycogen, lipid, protein) in cell periphery may be associated with the activation of mTORC1/mTORC2 (see Rabanal-Ruiz, Y. & Korolchuk, V. I. mTORC1 and Nutrient Homeostasis: The Central Role of the Lysosome. Int J Mol Sci 19, 818 (2018); Jia, R. & Bonifacino, J. S. Lysosome Positioning Influences mTORC2 and AKT Signaling. Molecular Cell 75, 26-38.e3 (2019)).

Without wishing to be bound by theory, it is contemplated that Rab1a DN (dominant negative form of Rab1a) may simulate lysosome peripheral distribution (from perinuclear region) through activating mTORC1/mTORC2 proteins inside the cells without the need of external (or extracellular) signal for their activation (to support lysosome peripheral distribution).

The Use of Rab1a^{GDP} or Rab1a^{GTP} in Increasing Dopaminergic Neuron Cell Viability and/or Prevent Dopaminergic Neuron Cell Death:

As described in detail herein, it has now been identified that treatment with a microautophagy-enhancing agent comprising a GDP-bound form of Rab1a, such as Rab1a^{S25N}, Rab1a^{N124I} (a mouse Rab1 sequence), Rab1a^{D41N}, Rab1a^{D47N}, or another dominant negative (DN) GDP-bound form of Rab1a, or one or more expressible nucleic acids encoding such a Rab1a^{GDP}, may be used to increase cell viability and/or prevent cell death in dopaminergic neurons.

As will be understood, in certain embodiments, methods as described herein may be in vitro methods, in vivo methods, or both.

In an embodiment, there is provided herein a method for reducing symptoms of Parkinson's disease, or for preventing or treating Parkinson's disease, in a subject in need thereof, said method comprising:
treating the subject with a GDP-bound form of Rab1a (Rab1a^{GDP}), one or more expressible nucleic acids encoding Rab1a^{GDP}, or a combination thereof;
thereby increasing cellular levels of Rab1a^{GDP} in the subject, resulting in decreased symptoms of Parkinson's disease the subject.

In an embodiment of the above method, the symptoms include at least one of the following: static tremors, postural imbalance, bradykinesia, muscle rigidity, punding, dyskinesias, hallucinations, impulse control disorders and sleep disorders.

In an embodiment, there is provided herein a method for increasing cell viability of a dopaminergic neuron, or for preventing or treating dopaminergic neuronal death, in a subject in need thereof, said method comprising:
treating a dopaminergic neuron of the subject with a GDP-bound form of Rab1a (Rab1a^{GDP}), one or more expressible nucleic acids encoding Rab1a^{GDP}, or a combination thereof;
thereby increasing a cellular level of Rab1a^{GDP} in the dopaminergic neuron, resulting in decreased neuronal death of the dopaminergic neuron.

In another embodiment, there is provided herein a method for increasing cell viability of a dopaminergic neuron in vitro or in vivo, said method comprising:
treating the dopaminergic neuron with a GDP-bound form of Rab1a (Rab1a^{GDP}), one or more expressible nucleic acids encoding Rab1a^{GDP}, or a combination thereof;
thereby increasing a cellular level of Rab1a^{GDP} in the dopaminergic neuron, resulting in increased cell viability or decreased neuronal death of the dopaminergic neuron.

In another embodiment of any of the above method or methods, the Rab1a^{GDP} may be or may comprise Rab1a^{S25N}, Rab1a^{N124I}, Rab1a^{D41N} Rab1a^{D47N}, or another dominant negative (DN) GDP-bound form of Rab1a.

In still another embodiment of any of the above method or methods, the Rab1a^{GDP} may comprise the amino acid sequence:

(SEQ ID NO: 6; Human Rab1a^{S25N})
MSSMNPEYDYLFKLLLIGDSGVGKNCLLLRFADDTYTESYISTIGVDFKI
RTIELDGKTIKLQIWDTAGQERFRTITSSYYRGAHGIIVVYDVTDQESFN
NVKQWLQEIDRYASENVNKLLVGNKCDLTTKKVVDYTTAKEFADSLGIPF
LETSAKNATNVEQSFMTMAAEIKKRMGPGATAGGAEKSNVKIQSTPVKQS
GGGCC;
(SEQ ID NO: 9; Mouse Rab1^{N124I})
MGDYKDDDDKGGSGGSSMNPEYDYLFKLLLIGDSGVGKSCLLLRFADDTY
TESYISTIGVDFKIRTIELDGKTIKLQIWDTAGQERFRTITSSYYRGAHG
IIVVYDVTDQESFNNVKQWLQEIDRYASENVNKLLVGIKCDLTTKKVVDY
TTAKEFADSLGIPFLETSAKNATNVEQSFMTMAAEIKKRMGPGATAGGAE
KSNVKIQSTPVKQSGGGC;

the amino acid sequence of human Rab1a^{D41N}, or

(SEQ ID NO: 18; Human Rab1a^{D47N})
MSSMNPEYDYLFKLLLIGDSGVGKSCLLLRFADDTYTESYISTIGVNFKI
RTIELDGKTIKLQIWDTAGQERFRTITSSYYRGAHGIIVVYDVTDQESFN
NVKQWLQEIDRYASENVNKLLVGNKCDLTTKKVVDYTTAKEFADSLGIPF
LETSAKNATNVEQSFMTMAAEIKKRMGPGATAGGAEKSNVKIQSTPVKQS
GGGCC;

or a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity with any of these sequences and preferentially binding GDP.

In yet another embodiment of any of the above method or methods, the Rab1a^{GDP} may consist of the amino acid sequence:

(SEQ ID NO: 6; Human Rab1a^{S25N})
MSSMNPEYDYLFKLLLIGDSGVGKNCLLLRFADDTYTESYISTIGVDFKI
RTIELDGKTIKLQIWDTAGQERFRTITSSYYRGAHGIIVVYDVTDQESFN
NVKQWLQEIDRYASENVNKLLVGNKCDLTTKKVVDYTTAKEFADSLGIPF
LETSAKNATNVEQSFMTMAAEIKKRMGPGATAGGAEKSNVKIQSTPVKQS
GGGCC;
(SEQ ID NO: 9; Mouse Rab1^{N1241})
MGDYKDDDDKGGSGGSSMNPEYDYLFKLLLIGDSGVGKSCLLLRFADDTY
TESYISTIGVDFKIRTIELDGKTIKLQIWDTAGQERFRTITSSYYRGAHG
IIVVYDVTDQESENNVKQWLQEIDRYASENVNKLLVGIKCDLTTKKVVDY
TTAKEFADSLGIPFLETSAKNATNVEQSFMTMAAEIKKRMGPGATAGGAE
KSNVKIQSTPVKQSGGGCC;

the amino acid sequence of human Rab1a^{D41N}; or

(SEQ ID NO: 18; Human Rab1a^{D47N})
MSSMNPEYDYLFKLLLIGDSGVGKSCLLLRFADDTYTESYISTIGVNFKI
RTIELDGKTIKLQIWDTAGQERFRTITSSYYRGAHGIIVVYDVTDQESFN
NVKQWLQEIDRYASENVNKLLVGNKCDLTTKKVVDYTTAKEFADSLGIPF
LETSAKNATNVEQSFMTMAAEIKKRMGPGATAGGAEKSNVKIQSTPVKQS
GGGCC.

or a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity with any of these sequences and preferentially binding GDP.

In another embodiment of any of the above method or methods, the Rab1a^{GDP} may comprise or consist of the amino acid sequence:

(SEQ ID NO: 6; Human Rab1a^{S25N})
MSSMNPEYDYLFKLLLIGDSGVGKNCLLLRFADDTYTESYISTIGVDFKI
RTIELDGKTIKLQIWDTAGQERFRTITSSYYRGAHGIIVVYDVTDQESFN
NVKQWLQEIDRYASENVNKLLVGNKCDLTTKKVVDYTTAKEFADSLGIPF
LETSAKNATNVEQSFMTMAAEIKKRMGPGATAGGAEKSNVKIQSTPVKQS
GGGCC;

or a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity therewith and preferentially binding GDP.

In still another embodiment of any of the above method or methods, the Rab1a^{GDP} may be in the form of a fusion protein, wherein the Rab1a^{GDP} is fused or otherwise directly or indirectly linked, optionally via a linker, with a signaling or targeting peptide, a fluorescent peptide or other marker or tracer, or another peptide or non-peptide moiety for targeted delivery, facilitating cell uptake, increasing stability or in vivo half-life, or improving another therapeutic, diagnostic, or in vivo property of the Rab1a^{GDP}. In an embodiment that is not meant to be limiting in any manner, the fusion protein can comprise any protein or tag, for example, GFP, YFP, mCherry, luciferase specific antibody, aptamer for identification or delivery to a specific organ or the like.

In yet another embodiment of any of the above method or methods, the fusion protein may comprise the amino acid sequence:

(SEQ ID NO: 6; Human Rab1a^{S25N})
MSSMNPEYDYLFKLLLIGDSGVGKNCLLLRFADDTYTESYISTIGVDFKI
RTIELDGKTIKLQIWDTAGQERFRTITSSYYRGAHGIIVVYDVTDQESFN
NVKQWLQEIDRYASENVNKLLVGNKCDLTTKKVVDYTTAKEFADSLGIPF
LETSAKNATNVEQSFMTMAAEIKKRMGPGATAGGAEKSNVKIQSTPVKQS
GGGCC;

or a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity therewith and preferentially binding GDP.

In another embodiment of any of the above method or methods, the Rab1a^{GDP} may be in the form of a fusion protein, and may comprise the amino acid sequence:

(SEQ ID NO: 21)
MEDAKNIKKGPAPFYPLEDGTAGEQLHKAMKRYALVPGTIAFTDAHIEVN
ITYAEYFEMSVRLAEAMKRYGLNTNHRIVVCSENSLQFFMPVLGALFIGV
AVAPANDIYNERELLNSMNISQPTVVFVSKKGLQKILNVQKKLPIIQKII
IMDSKTDYQGFQSMYTFVTSHLPPGFNEYDFVPESFDRDKTIALIMNSSG
STGLPKGVALPHRTACVRFSHARDPIFGNQIIPDTAILSVVPFHHGFGMF
TTLGYLICGFRVVLMYRFEEELFLRSLQDYKIQSALLVPTLFSFFAKSTL
IDKYDLSNLHEIASGGAPLSKEVGEAVAKRFHLPGIRQGYGLTETTSAIL
ITPEGDDKPGAVGKVVPFFEAKVVDLDTGKTLGVNQRGELCVRGPMIMSG
YVNNPEATNALIDKDGWLHSGDIAYWDEDEHFFIVDRLKSLIKYKGYQVA
PAELESILLQHPNIFDAGVAGLPDDDAGELPAAVVVLEHGKTMTEKEIVD
YVASQVTTAKKLRGGVVFVDEVPKGLTGKLDARKIREILIKAKKGGKSKL
MSSMNPEYDYLFKLLLIGDSGVGKNCLLLRFADDTYTESYISTIGVDFKI
RTIELDGKTIKLQIWDTAGQERFRTITSSYYRGAHGIIVVYDVTDQESFN
NVKQWLQEIDRYASENVNKLLVGNKCDLTTKKVVDYTTAKEFADSLGIPF
LETSAKNATNVEQSFMTMAAEIKKRMGPGATAGGAEKSNVKIQSTPVKQS
GGGCC;

or a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity therewith and preferentially binding GDP.

In still another embodiment of any of the above method or methods, the one or more expressible nucleic acids may encode any one or more of the Rab1a^{GDP}s as defined herein.

In yet another embodiment of any of the above method or methods, the one or more expressible nucleic acids may be DNA-based, or RNA-based.

In another embodiment of any of the above method or methods, the one or more expressible nucleic acids may transiently express the Rab1a^{GDP} in the cell, or wherein the one or more expressible nucleic acids may integrate in the cell genome and express the Rab1a^{GDP} in the cell.

In still another embodiment of any of the above method or methods, the one or more expressible nucleic acids may comprise one or more expression vectors, plasmids, or mRNAs encoding and capable of expressing the Rab1a^{GDP} inside the cell.

In yet another embodiment of any of the above method or methods, the one or more expressible nucleic acids may comprise a nucleic acid sequence of:

(Human Rab1a^{S25N} ORF Codon Sequence, SEQ ID NO: 5)
ATGTCCAGCATGAATCCCGAATATGATTATTTATTCAAGTTACTTCTGAT
TGGCGACTCAGGGGTTGGAAAGAATTGCCTTCTTCTTAGGTTTGCAGATG
ATACATATACAGAAAGCTACATCAGCACAATTGGTGTGGATTTCAAAATA
AGAACTATAGAGTTAGACGGGAAAACAATCAAGCTTCAAATATGGGACAC
AGCAGGCCAGGAAAGATTTCGAACAATCACCTCCAGTTATTACAGAGGAG
CCCATGGCATCATAGTTGTGTATGATGTGACAGATCAGGAGTCCTTCAAT
AATGTTAAACAGTGGCTGCAGGAAATAGATCGTTATGCCAGTGAAAATGT
CAACAAATTGTTGGTAGGGAACAAATGTGATCTGACCACAAAGAAAGTAG
TAGACTACACAACAGCGAAGGAATTTGCTGATTCCCTTGGAATTCCGTTT
TTGGAAACCAGTGCTAAGAATGCAACGAATGTAGAACAGTCTTTCATGAC
GATGGCAGCTGAGATTAAAAAGCGAATGGGTCCCGGAGCAACAGCTGGTG
GTGCTGAGAAGTCCAATGTTAAAATTCAGAGCACTCCAGTCAAGCAGTCA
GGTGGAGGTTGCTGCTAA;
(Mouse Rab1a^{N124I} ORF Codon Seqeunce, SEQ ID NO: 8)
ATGGGGGACTACAAGGACGACGATGACAAGGGGGGTAGCGGTGGATCCAG
CATGAATCCCGAATATGATTATTTATTCAAGTTACTTCTGATTGGCGATT
CTGGGGTTGGAAAGTCCTGCCTTCTCCTTAGGTTTGCAGATGATACGTAT
ACGGAAAGCTACATCAGCACAATTGGTGTGGATTTCAAGATACGAACTAT
AGAGTTAGATGGGAAAACAATCAAGCTACAGATATGGGACACAGCAGGCC
AGGAAAGATTTCGAACAATCACTTCCAGTTATTACAGAGGAGCCCATGGC
ATCATAGTTGTGTATGATGTGACAGATCAGGAGTCCTTCAATAACGTTAA
ACAGTGGCTGCAGGAGATAGATCGCTACGCCAGTGAAAATGTCAACAAGT
TGTTGGTAGGGATCAAATGTGACCTGACCACAAAGAAAGTAGTAGACTAC
ACAACAGCAAAGGAATTTGCAGATTCCCTTGGAATTCCATTTTTGGAAAC
CAGTGCTAAGAACGCAACGAATGTAGAACAGTCTTTCATGACGATGGCAG
CTGAGATTAAAAAGCGAATGGGTCCTGGAGCTACAGCTGGTGGTGCCGAG
AAGTCCAATGTTAAAATCCAGAGCACTCCAGTCAAGCAGTCAGGTGGAGG
CTGCTGCTAA;
or
(Human Rab1a^{D47N} ORF Codon Sequence, SEQ ID NO: 17)
ATGTCCAGCATGAATCCCGAATATGATTATTTATTCAAGTTACTTCTGAT
TGGCGACTCAGGGGTTGGAAAGTCTTGCCTTCTTCTTAGGTTTGCAGATG
ATACATATACAGAAAGCTACATCAGCACAATTGGTGTGAACTTCAAAATA
AGAACTATAGAGTTAGACGGGAAAACAATCAAGCTTCAAATATGGGACAC
AGCAGGCCAGGAAAGATTTCGAACAATCACCTCCAGTTATTACAGAGGAG
CCCATGGCATCATAGTTGTGTATGATGTGACAGATCAGGAGTCCTTCAAT
AATGTTAAACAGTGGCTGCAGGAAATAGATCGTTATGCCAGTGAAAATGT
CAACAAATTGTTGGTAGGGAACAAATGTGATCTGACCACAAAGAAAGTAG
TAGACTACACAACAGCGAAGGAATTTGCTGATTCCCTTGGAATTCCGTTT
TTGGAAACCAGTGCTAAGAATGCAACGAATGTAGAACAGTCTTTCATGAC
GATGGCAGCTGAGATTAAAAAGCGAATGGGTCCCGGAGCAACAGCTGGTG
GTGCTGAGAAGTCCAATGTTAAAATTCAGAGCACTCCAGTCAAGCAGTCA
GGTGGAGGTTGCTGCTAA;

or a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity therewith and encoding a Rab1a^{GDP} preferentially binding GDP;
or a nucleic acid sequence equivalent to any of the above sequences due to codon redundancy.

In another embodiment of any of the above method or methods, the one or more expressible nucleic acids may comprise a nucleic acid sequence of:

(SEQ ID NO: 19, MG-008 ORF DNA Sequence with 5′
Luciferase Tag);
ATGGAAGACGCCAAAAACATAAAGAAAGGCCCGGCGCCATTCTATCCGCT
AGAGGATGGAACCGCTGGAGAGCAACTGCATAAGGCTATGAAGAGATACG
CCCTGGTTCCTGGAACAATTGCTTTTACAGATGCACATATCGAGGTGAAC
ATCACGTACGCGGAATACTTCGAAATGTCCGTTCGGTTGGCAGAAGCTAT
GAAACGATATGGGCTGAATACAAATCACAGAATCGTCGTATGCAGTGAAA
ACTCTCTTCAATTCTTTATGCCGGTGTTGGGCGCGTTATTTATCGGAGTT
GCAGTTGCGCCCGCGAACGACATTTATAATGAACGTGAATTGCTCAACAG
TATGAACATTTCGCAGCCTACCGTAGTGTTTGTTTCCAAAAAGGGGTTGC
AAAAAATTTTGAACGTGCAAAAAAAATTACCAATAATCCAGAAAATTATT
ATCATGGATTCTAAAACGGATTACCAGGGATTTCAGTCGATGTACACGTT
CGTCACATCTCATCTACCTCCCGGTTTTAATGAATACGATTTTGTACCAG
AGTCCTTTGATCGTGACAAAACAATTGCACTGATAATGAACTCCTCTGGA
TCTACTGGGTTACCTAAGGGTGTGGCCCTTCCGCATAGAACTGCCTGCGT
CAGATTCTCGCATGCCAGAGATCCTATTTTTGGCAATCAAATCATTCCGG
ATACTGCGATTTTAAGTGTTGTTCCATTCCATCACGGTTTTGGAATGTTT
ACTACACTCGGATATTTGATATGTGGATTTCGAGTCGTCTTAATGTATAG
ATTTGAAGAAGAGCTGTTTTTACGATCCCTTCAGGATTACAAAATTCAAA
GTGCGTTGCTAGTACCAACCCTATTTTCATTCTTCGCCAAAAGCACTCTG
ATTGACAAATACGATTTATCTAATTTACACGAAATTGCTTCTGGGGGCGC
ACCTCTTTCGAAAGAAGTCGGGGAAGCGGTTGCAAAACGCTTCCATCTTC
CAGGGATACGACAAGGATATGGGCTCACTGAGACTACATCAGCTATTCTG
ATTACACCCGAGGGGGATGATAAACCGGGCGCGGTCGGTAAAGTTGTTCC
ATTTTTTGAAGCGAAGGTTGTGGATCTGGATACCGGGAAAACGCTGGGCG
TTAATCAGAGAGGCGAATTATGTGTCAGAGGACCTATGATTATGTCCGGT
TATGTAAACAATCCGGAAGCGACCAACGCCTTGATTGACAAGGATGGATG
GCTACATTCTGGAGACATAGCTTACTGGGACGAAGACGAACACTTCTTCA
TAGTTGACCGCTTGAAGTCTTTAATTAAATACAAAGGATACCAGGTGGCC
CCCGCTGAATTGGAGTCGATATTGTTACAACACCCCAACATCTTCGACGC
GGGCGTGGCAGGTCTTCCCGACGATGACGCCGGTGAACTTCCCGCCGCCG
TTGTTGTTTTGGAGCACGGAAAGACGATGACGGAAAAAGAGATCGTGGAT
TACGTCGCCAGTCAAGTAACAACCGCCAAAAAGTTGCGCGGAGGAGTTGT
GTTTGTGGACGAAGTACCGAAAGGTCTTACCGGAAAACTCGACGCAAGAA
AAATCAGAGAGATCCTCATAAAGGCCAAGAAGGGCGGAAAGTCCAAATTG
ATGTCCAGCATGAATCCCGAATATGATTATTTATTCAAGTTACTTCTGAT
TGGCGACTCAGGGGTTGGAAAGAATTGCCTTCTTCTTAGGTTTGCAGATG
ATACATATACAGAAAGCTACATCAGCACAATTGGTGTGGATTTCAAAATA
AGAACTATAGAGTTAGACGGGAAAACAATCAAGCTTCAAATATGGGACAC
AGCAGGCCAGGAAAGATTTCGAACAATCACCTCCAGTTATTACAGAGGAG
CCCATGGCATCATAGTTGTGTATGATGTGACAGATCAGGAGTCCTTCAAT
AATGTTAAACAGTGGCTGCAGGAAATAGATCGTTATGCCAGTGAAAATGT
CAACAAATTGTTGGTAGGGAACAAATGTGATCTGACCACAAAGAAAGTAG
TAGACTACACAACAGCGAAGGAATTTGCTGATTCCCTTGGAATTCCGTTT
TTGGAAACCAGTGCTAAGAATGCAACGAATGTAGAACAGTCTTTCATGAC
GATGGCAGCTGAGATTAAAAAGCGAATGGGTCCCGGAGCAACAGCTGGTG
GTGCTGAGAAGTCCAATGTTAAAATTCAGAGCACTCCAGTCAAGCAGTCA
GGTGGAGGTTGCTGCTAA
or
(SEQ ID NO: 20, MG-008 ORF mRNA Sequence with 5′
Luciferase Tag)
AUGGAAGACGCCAAAAACAUAAAGAAAGGCCCGGCGCCAUUCUAUCCGCUA
GAGGAUGGAACCGCUGGAGAGCAACUGCAUAAGGCUAUGAAGAGAUACGCC
CUGGUUCCUGGAACAAUUGCUUUUACAGAUGCACAUAUCGAGGUGAACAU
CACGUACGCGGAAUACUUCGAAAUGUCCGUUCGGUUGGCAGAAGCUAUGA
AACGAUAUGGGCUGAAUACAAAUCACAGAAUCGUCGUAUGCAGUGAAAAC
UCUCUUCAAUUCUUUAUGCCGGUGUUGGGCGCGUUAUUUAUCGGAGUUGC
AGUUGCGCCCGCGAACGACAUUUAUAAUGAACGUGAAUUGCUCAACAGUA
UGAACAUUUCGCAGCCUACCGUAGUGUUUGUUUCCAAAAAGGGGUUGCAA
AAAAUUUUGAACGUGCAAAAAAAAUUACCAAUAAUCCAGAAAAUUAUUAU
CAUGGAUUCUAAAACGGAUUACCAGGGAUUUCAGUCGAUGUACACGUUCG
UCACAUCUCAUCUACCUCCCGGUUUUAAUGAAUACGAUUUUGUACCAGAG
UCCUUUGAUCGUGACAAAACAAUUGCACUGAUAAUGAACUCCUCUGGAUC
UACUGGGUUACCUAAGGGUGUGGCCCUUCCGCAUAGAACUGCCUGCGUCA
GAUUCUCGCAUGCCAGAGAUCCUAUUUUUGGCAAUCAAAUCAUUCCGGAU
ACUGCGAUUUUAAGUGUUGUUCCAUUCCAUCACGGUUUUGGAAUGUUUAC
UACACUCGGAUAUUUGAUAUGUGGAUUUCGAGUCGUCUUAAUGUAUAGAU
UUGAAGAAGAGCUGUUUUUACGAUCCCUUCAGGAUUACAAAAUUCAAAGU
GCGUUGCUAGUACCAACCCUAUUUUCAUUCUUCGCCAAAAGCACUCUGAU
UGACAAAUACGAUUUAUCUAAUUUACACGAAAUUGCUUCUGGGGGCGCAC
CUCUUUCGAAAGAAGUCGGGGAAGCGGUUGCAAAACGCUUCCAUCUUCCA
GGGAUACGACAAGGAUAUGGGCUCACUGAGACUACAUCAGCUAUUCUGAU
UACACCCGAGGGGGAUGAUAAACCGGGCGCGGUCGGUAAAGUUGUUCCAU
UUUUUGAAGCGAAGGUUGUGGAUCUGGAUACCGGGAAAACGCUGGGCGUU
AAUCAGAGAGGCGAAUUAUGUGUCAGAGGACCUAUGAUUAUGUCCGGUUA
UGUAAACAAUCCGGAAGCGACCAACGCCUUGAUUGACAAGGAUGGAUGGC
UACAUUCUGGAGACAUAGCUUACUGGGACGAAGACGAACACUUCUUCAUA
GUUGACCGCUUGAAGUCUUUAAUUAAAUACAAAGGAUACCAGGUGGCCCC
CGCUGAAUUGGAGUCGAUAUUGUUACAACACCCCAACAUCUUCGACGCGG
GCGUGGCAGGUCUUCCCGACGAUGACGCCGGUGAACUUCCCGCCGCCGUU
GUUGUUUUGGAGCACGGAAAGACGAUGACGGAAAAAGAGAUCGUGGAUUA
CGUCGCCAGUCAAGUAACAACCGCCAAAAAGUUGCGCGGAGGAGUUGUGU
UUGUGGACGAAGUACCGAAAGGUCUUACCGGAAAACUCGACGCAAGAAAA
AUCAGAGAGAUCCUCAUAAAGGCCAAGAAGGGCGGAAAGUCCAAAUUGAU
GUCCAGCAUGAAUCCCGAAUAUGAUUAUUUAUUCAAGUUACUUCUGAUUG
GCGACUCAGGGGUUGGAAAGAAUUGCCUUCUUCUUAGGUUUGCAGAUGAU
ACAUAUACAGAAAGCUACAUCAGCACAAUUGGUGUGGAUUUCAAAAUAAG
AACUAUAGAGUUAGACGGGAAAACAAUCAAGCUUCAAAUAUGGGACACAG
CAGGCCAGGAAAGAUUUCGAACAAUCACCUCCAGUUAUUACAGAGGAGCC
CAUGGCAUCAUAGUUGUGUAUGAUGUGACAGAUCAGGAGUCCUUCAAUAA
UGUUAAACAGUGGCUGCAGGAAAUAGAUCGUUAUGCCAGUGAAAAUGUCA
ACAAAUUGUUGGUAGGGAACAAAUGUGAUCUGACCACAAAGAAAGUAGUA
GACUACACAACAGCGAAGGAAUUUGCUGAUUCCCUUGGAAUUCCGUUUUU
GGAAACCAGUGCUAAGAAUGCAACGAAUGUAGAACAGUCUUUCAUGACGA
UGGCAGCUGAGAUUAAAAAGCGAAUGGGUCCCGGAGCAACAGCUGGUGGU
GCUGAGAAGUCCAAUGUUAAAAUUCAGAGCACUCCAGUCAAGCAGUCAGG
UGGAGGUUGCUGCUAA;

or a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity therewith and encoding a Rab1a^{GDP} preferentially binding GDP;
or a nucleic acid sequence equivalent to any of the above sequences due to codon redundancy.

In another embodiment, there is provided herein a use of a GDP-bound form of Rab1a (Rab1a^{GDP}), one or more expressible nucleic acids encoding Rab1a^{GDP}, or a combination thereof, for reducing symptoms of Parkinson's disease in a subject, or for preventing or treating Parkinson's disease, in a subject in need thereof.

In an embodiment of the above use, the symptoms include at least one of the following: static tremor, postural imbalance, bradykinesia, muscle rigidity, punding, dyskinesias, hallucinations, impulse control disorders and sleep disorders.

In another embodiment, there is provided herein a use of a GDP-bound form of Rab1a (Rab1a^{GDP}), one or more expressible nucleic acids encoding Rab1a^{GDP}, or a combination thereof, for increasing cell viability of a dopaminergic neuron, or for preventing or treating dopaminergic neuronal death, in a subject in need thereof.

In another embodiment, there is provided herein a use of a GDP-bound form of Rab1a (Rab1a^{GDP}), one or more expressible nucleic acids encoding Rab1a^{GDP}, or a combination thereof, for increasing cell viability of a dopaminergic neuron in vitro or in vivo.

In another embodiment, there is provided herein a use of a GDP-bound form of Rab1a (Rab1a^{GDP}), one or more expressible nucleic acids encoding Rab1a^{GDP}, or a combination thereof, in the manufacture of a medicament for reducing symptoms of Parkinson's disease in a subject, or for preventing or treating Parkinson's disease, in a subject in need thereof.

In an embodiment of the above use, the symptoms include at least one of the following: static tremor, postural imbalance, bradykinesia, muscle rigidity, punding, dyskinesias, hallucinations, impulse control disorders and sleep disorders.

In another embodiment, there is provided herein a use of a GDP-bound form of Rab1a (Rab1a^{GDP}), one or more expressible nucleic acids encoding Rab1a^{GDP}, or a combination thereof, in the manufacture of a medicament for increasing cell viability of a dopaminergic neuron, or for preventing or treating dopaminergic neuronal death, in a subject in need thereof.

In another embodiment, there is provided herein a use of a GDP-bound form of Rab1a (Rab1a^{GDP}), one or more expressible nucleic acids encoding Rab1a^{GDP}, or a combination thereof, in the manufacture of a medicament for increasing cell viability of a dopaminergic neuron in vitro or in vivo.

In another embodiment of any of the above use or uses, the Rab1a^{GDP} may be or may comprise Rab1a^{S25N}, Rab1a^{N124I}, Rab1a^{D41N}, Rab1a^{D47N}, or another dominant negative (DN) GDP-bound form of Rab1a.

In still another embodiment of any of the above use or uses, the Rab1a^{GDP} may comprise the amino acid sequence:

(SEQ ID NO: 6; Human Rab1a^{S25N})
MSSMNPEYDYLFKLLLIGDSGVGKNCLLLRFADDTYTESYISTIGVDFKI
RTIELDGKTIKLQIWDTAGQERFRTITSSYYRGAHGIIVVYDVTDQESFN
NVKQWLQEIDRYASENVNKLLVGNKCDLTTKKVVDYTTAKEFADSLGIPF
LETSAKNATNVEQSFMTMAAEIKKRMGPGATAGGAEKSNVKIQSTPVKQS
GGGCC;
(SEQ ID NO: 9; Mouse Rab1^{N124I})
MGDYKDDDDKGGSGGSSMNPEYDYLFKLLLIGDSGVGKSCLLLRFADDTY
TESYISTIGVDFKIRTIELDGKTIKLQIWDTAGQERFRTITSSYYRGAHG
IIVVYDVTDQESFNNVKQWLQEIDRYASENVNKLLVGIKCDLTTKKVVDY
TTAKEFADSLGIPFLETSAKNATNVEQSFMTMAAEIKKRMGPGATAGGAE
KSNVKIQSTPVKQSGGGCC;

the amino acid sequence of human Rab1a^{D41N}; or

(SEQ ID NO: 18; Human Rab1a^{D47N})
MSSMNPEYDYLFKLLLIGDSGVGKSCLLLRFADDTYTESYISTIGVNFKI
RTIELDGKTIKLQIWDTAGQERFRTITSSYYRGAHGIIVVYDVTDQESFN
NVKQWLQEIDRYASENVNKLLVGNKCDLTTKKVVDYTTAKEFADSLGIPF
LETSAKNATNVEQSFMTMAAEIKKRMGPGATAGGAEKSNVKIQSTPVKQS
GGGCC;

or a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity with any of these sequences and preferentially binding GDP.

In yet another embodiment of any of the above use or uses, the Rab1a^{GDP} may consist of the amino acid sequence:

(SEQ ID NO: 6; Human Rab1a^{S25N})
MSSMNPEYDYLFKLLLIGDSGVGKNCLLLRFADDTYTESYISTIGVDFKI
RTIELDGKTIKLQIWDTAGQERFRTITSSYYRGAHGIIVVYDVTDQESFN
NVKQWLQEIDRYASENVNKLLVGNKCDLTTKKVVDYTTAKEFADSLGIPF
LETSAKNATNVEQSFMTMAAEIKKRMGPGATAGGAEKSNVKIQSTPVKQS
GGGCC;
(SEQ ID NO: 9; Mouse Rab1N1241)
MGDYKDDDDKGGSGGSSMNPEYDYLFKLLLIGDSGVGKSCLLLRFADDTY
TESYISTIGVDFKIRTIELDGKTIKLQIWDTAGQERFRTITSSYYRGAHG
IIVVYDVTDQESENNVKQWLQEIDRYASENVNKLLVGIKCDLTTKKVVDY
TTAKEFADSLGIPFLETSAKNATNVEQSFMTMAAEIKKRMGPGATAGGAE
KSNVKIQSTPVKQSGGGCC;

the amino acid sequence of human Rab1a^{D41N}; or

(SEQ ID NO: 18; Human Rab1a^{D47N})
MSSMNPEYDYLFKLLLIGDSGVGKSCLLLRFADDTYTESYISTIGVNFKI
RTIELDGKTIKLQIWDTAGQERFRTITSSYYRGAHGIIVVYDVTDQESFN
NVKQWLQEIDRYASENVNKLLVGNKCDLTTKKVVDYTTAKEFADSLGIPF
LETSAKNATNVEQSFMTMAAEIKKRMGPGATAGGAEKSNVKIQSTPVKQS
GGGCC;

or a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity with any of these sequences and preferentially binding GDP.

In another embodiment of any of the above use or uses, the Rab1a^{GDP} may comprise or consist of the amino acid sequence:

(SEQ ID NO: 6; Human Rab1a^{S25N})
MSSMNPEYDYLFKLLLIGDSGVGKNCLLLRFADDTYTESYISTIGVDFKI
RTIELDGKTIKLQIWDTAGQERFRTITSSYYRGAHGIIVVYDVTDQESEN
NVKQWLQEIDRYASENVNKLLVGNKCDLTTKKVVDYTTAKEFADSLGIPF
LETSAKNATNVEQSFMTMAAEIKKRMGPGATAGGAEKSNVKIQSTPVKQS
GGGCC;

or a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity therewith and preferentially binding GDP.

In still another embodiment of any of the above use or uses, the Rab1a^{GDP} may be in the form of a fusion protein, wherein the Rab1a^{GDP} is fused or otherwise directly or indirectly linked, optionally via a linker, with a signaling or targeting peptide, a fluorescent peptide or other marker or tracer, or another peptide or non-peptide moiety for targeted delivery, facilitating cell uptake, increasing stability or in vivo half-life, or improving another therapeutic, diagnostic, or in vivo property of the Rab1a^{GDP}.

In yet another embodiment of any of the above use or uses, the fusion protein may comprise the amino acid sequence:

(SEQ ID NO: 6; Human Rab1a^{S25N})
MSSMNPEYDYLFKLLLIGDSGVGKNCLLLRFADDTYTESYISTIGVDFKI
RTIELDGKTIKLQIWDTAGQERFRTITSSYYRGAHGIIVVYDVTDQESFN
NVKQWLQEIDRYASENVNKLLVGNKCDLTTKKVVDYTTAKEFADSLGIPF
LETSAKNATNVEQSFMTMAAEIKKRMGPGATAGGAEKSNVKIQSTPVKQS
GGGCC;

or a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity therewith and preferentially binding GDP.

In another embodiment of any of the above use or uses, the Rab1a^{GDP} may be in the form of a fusion protein, and may comprise the amino acid sequence:

(SEQ ID NO: 21)
MEDAKNIKKGPAPFYPLEDGTAGEQLHKAMKRYALVPGTIAFTDAHIEVN
ITYAEYFEMSVRLAEAMKRYGLNTNHRIVVCSENSLQFFMPVLGALFIGV
AVAPANDIYNERELLNSMNISQPTVVFVSKKGLQKILNVQKKLPIIQKII
IMDSKTDYQGFQSMYTFVTSHLPPGFNEYDFVPESFDRDKTIALIMNSSG
STGLPKGVALPHRTACVRFSHARDPIFGNQIIPDTAILSVVPFHHGFGMF
TTLGYLICGFRVVLMYRFEEELFLRSLQDYKIQSALLVPTLFSFFAKSTL
IDKYDLSNLHEIASGGAPLSKEVGEAVAKRFHLPGIRQGYGLTETTSAIL
ITPEGDDKPGAVGKVVPFFEAKVVDLDTGKTLGVNQRGELCVRGPMIMSG
YVNNPEATNALIDKDGWLHSGDIAYWDEDEHFFIVDRLKSLIKYKGYQVA
PAELESILLQHPNIFDAGVAGLPDDDAGELPAAVVVLEHGKTMTEKEIVD
YVASQVTTAKKLRGGVVFVDEVPKGLTGKLDARKIREILIKAKKGGKSKL
MSSMNPEYDYLFKLLLIGDSGVGKNCLLLRFADDTYTESYISTIGVDFKI
RTIELDGKTIKLQIWDTAGQERFRTITSSYYRGAHGIIVVYDVTDQESFN
NVKQWLQEIDRYASENVNKLLVGNKCDLTTKKVVDYTTAKEFADSLGIPF
LETSAKNATNVEQSFMTMAAEIKKRMGPGATAGGAEKSNVKIQSTPVKQS
GGGCC;

or a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity therewith and preferentially binding GDP.

In still another embodiment of any of the above use or uses, the one or more expressible nucleic acids may encode one or more Rab1a^{GDP}s as defined herein.

In yet another embodiment of any of the above use or uses, the one or more expressible nucleic acids may be DNA-based, or RNA-based.

In another embodiment of any of the above use or uses, the one or more expressible nucleic acids may transiently express the Rab1a^{GDP} in the dopaminergic neuron, or wherein the one or more expressible nucleic acids may integrate in the cell genome and express the Rab1a^{GDP} in the cell.

In another embodiment of any of the above use or uses, the one or more expressible nucleic acids may comprise one or more expression vectors, plasmids, or mRNAs encoding and capable of expressing the Rab1a^{GDP} inside the cell.

In another embodiment of any of the above use or uses, the one or more expressible nucleic acids may comprise a nucleic acid sequence of:

(Human Rab1a^{S25N} ORF Codon Sequence, SEQ ID NO: 5)
ATGTCCAGCATGAATCCCGAATATGATTATTTATTCAAGTTACTTCTGATTGGCG
ACTCAGGGGTTGGAAAGAATTGCCTTCTTCTTAGGTTTGCAGATGATACATATAC
AGAAAGCTACATCAGCACAATTGGTGTGGATTTCAAAATAAGAACTATAGAGTT
AGACGGGAAAACAATCAAGCTTCAAATATGGGACACAGCAGGCCAGGAAAGAT
TTCGAACAATCACCTCCAGTTATTACAGAGGAGCCCATGGCATCATAGTTGTGTA
TGATGTGACAGATCAGGAGTCCTTCAATAATGTTAAACAGTGGCTGCAGGAAAT
AGATCGTTATGCCAGTGAAAATGTCAACAAATTGTTGGTAGGGAACAAATGTGA
TCTGACCACAAAGAAAGTAGTAGACTACACAACAGCGAAGGAATTTGCTGATTC
CCTTGGAATTCCGTTTTTGGAAACCAGTGCTAAGAATGCAACGAATGTAGAACA
GTCTTTCATGACGATGGCAGCTGAGATTAAAAAGCGAATGGGTCCCGGAGCAAC
AGCTGGTGGTGCTGAGAAGTCCAATGTTAAAATTCAGAGCACTCCAGTCAAGCA
GTCAGGTGGAGGTTGCTGCTAA;
(Mouse Rab1a^{N124I} ORF Codon Seqeunce, SEQ ID NO: 8)
ATGGGGGACTACAAGGACGACGATGACAAGGGGGGTAGCGGTGGATCCAGCAT
GAATCCCGAATATGATTATTTATTCAAGTTACTTCTGATTGGCGATTCTGGGGTT
GGAAAGTCCTGCCTTCTCCTTAGGTTTGCAGATGATACGTATACGGAAAGCTACA
TCAGCACAATTGGTGTGGATTTCAAGATACGAACTATAGAGTTAGATGGGAAAA
CAATCAAGCTACAGATATGGGACACAGCAGGCCAGGAAAGATTTCGAACAATCA
CTTCCAGTTATTACAGAGGAGCCCATGGCATCATAGTTGTGTATGATGTGACAGA
TCAGGAGTCCTTCAATAACGTTAAACAGTGGCTGCAGGAGATAGATCGCTACGC
CAGTGAAAATGTCAACAAGTTGTTGGTAGGGATCAAATGTGACCTGACCACAAA
GAAAGTAGTAGACTACACAACAGCAAAGGAATTTGCAGATTCCCTTGGAATTCC
ATTTTTGGAAACCAGTGCTAAGAACGCAACGAATGTAGAACAGTCTTTCATGAC
GATGGCAGCTGAGATTAAAAAGCGAATGGGTCCTGGAGCTACAGCTGGTGGTGC
CGAGAAGTCCAATGTTAAAATCCAGAGCACTCCAGTCAAGCAGTCAGGTGGAGG
CTGCTGCTAA;
or
(Human Rab1a^{DA7N} ORF Codon Sequence, SEQ ID NO: 17)
ATGTCCAGCATGAATCCCGAATATGATTATTTATTCAAGTTACTTCTGATTGGCGACT
CAGGGGTTGGAAAGTCTTGCCTTCTTCTTAGGTTTGCAGATGATACATATACAGAAA
GCTACATCAGCACAATTGGTGTGAACTTCAAAATAAGAACTATAGAGTTAGACGGG
AAAACAATCAAGCTTCAAATATGGGACACAGCAGGCCAGGAAAGATTTCGAACAAT
CACCTCCAGTTATTACAGAGGAGCCCATGGCATCATAGTTGTGTATGATGTGACAGA
TCAGGAGTCCTTCAATAATGTTAAACAGTGGCTGCAGGAAATAGATCGTTATGCCAG
TGAAAATGTCAACAAATTGTTGGTAGGGAACAAATGTGATCTGACCACAAAGAAAG
TAGTAGACTACACAACAGCGAAGGAATTTGCTGATTCCCTTGGAATTCCGTTTTTGG
AAACCAGTGCTAAGAATGCAACGAATGTAGAACAGTCTTTCATGACGATGGCAGCT
GAGATTAAAAAGCGAATGGGTCCCGGAGCAACAGCTGGTGGTGCTGAGAAGTCCAA
TGTTAAAATTCAGAGCACTCCAGTCAAGCAGTCAGGTGGAGGTTGCTGCTAA;

or a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity therewith and encoding a Rab1a^{GDP} preferentially binding GDP;
or a nucleic acid sequence equivalent to any of the above sequences due to codon redundancy.

In still another embodiment of any of the above use or uses, the one or more expressible nucleic acids may comprise a nucleic acid sequence of:

(SEQ ID NO: 19, MG-008 ORF DNA Sequence with 5′
Luciferase Tag)
ATGGAAGACGCCAAAAACATAAAGAAAGGCCCGGCGCCATTCTATCCGCT
AGAGGATGGAACCGCTGGAGAGCAACTGCATAAGGCTATGAAGAGATACG
CCCTGGTTCCTGGAACAATTGCTTTTACAGATGCACATATCGAGGTGAAC
ATCACGTACGCGGAATACTTCGAAATGTCCGTTCGGTTGGCAGAAGCTAT
GAAACGATATGGGCTGAATACAAATCACAGAATCGTCGTATGCAGTGAAA
ACTCTCTTCAATTCTTTATGCCGGTGTTGGGCGCGTTATTTATCGGAGTT
GCAGTTGCGCCCGCGAACGACATTTATAATGAACGTGAATTGCTCAACAG
TATGAACATTTCGCAGCCTACCGTAGTGTTTGTTTCCAAAAAGGGGTTGC
AAAAAATTTTGAACGTGCAAAAAAAATTACCAATAATCCAGAAAATTATT
ATCATGGATTCTAAAACGGATTACCAGGGATTTCAGTCGATGTACACGTT
CGTCACATCTCATCTACCTCCCGGTTTTAATGAATACGATTTTGTACCAG
AGTCCTTTGATCGTGACAAAACAATTGCACTGATAATGAACTCCTCTGGA
TCTACTGGGTTACCTAAGGGTGTGGCCCTTCCGCATAGAACTGCCTGCGT
CAGATTCTCGCATGCCAGAGATCCTATTTTTGGCAATCAAATCATTCCGG
ATACTGCGATTTTAAGTGTTGTTCCATTCCATCACGGTTTTGGAATGTTT
ACTACACTCGGATATTTGATATGTGGATTTCGAGTCGTCTTAATGTATAG
ATTTGAAGAAGAGCTGTTTTTACGATCCCTTCAGGATTACAAAATTCAAA
GTGCGTTGCTAGTACCAACCCTATTTTCATTCTTCGCCAAAAGCACTCTG
ATTGACAAATACGATTTATCTAATTTACACGAAATTGCTTCTGGGGGCGC
ACCTCTTTCGAAAGAAGTCGGGGAAGCGGTTGCAAAACGCTTCCATCTTC
CAGGGATACGACAAGGATATGGGCTCACTGAGACTACATCAGCTATTCTG
ATTACACCCGAGGGGGATGATAAACCGGGCGCGGTCGGTAAAGTTGTTCC
ATTTTTTGAAGCGAAGGTTGTGGATCTGGATACCGGGAAAACGCTGGGCG
TTAATCAGAGAGGCGAATTATGTGTCAGAGGACCTATGATTATGTCCGGT
TATGTAAACAATCCGGAAGCGACCAACGCCTTGATTGACAAGGATGGATG
GCTACATTCTGGAGACATAGCTTACTGGGACGAAGACGAACACTTCTTCA
TAGTTGACCGCTTGAAGTCTTTAATTAAATACAAAGGATACCAGGTGGCC
CCCGCTGAATTGGAGTCGATATTGTTACAACACCCCAACATCTTCGACGC
GGGCGTGGCAGGTCTTCCCGACGATGACGCCGGTGAACTTCCCGCCGCCG
TTGTTGTTTTGGAGCACGGAAAGACGATGACGGAAAAAGAGATCGTGGAT
TACGTCGCCAGTCAAGTAACAACCGCCAAAAAGTTGCGCGGAGGAGTTGT
GTTTGTGGACGAAGTACCGAAAGGTCTTACCGGAAAACTCGACGCAAGAA
AAATCAGAGAGATCCTCATAAAGGCCAAGAAGGGCGGAAAGTCCAAATTG
ATGTCCAGCATGAATCCCGAATATGATTATTTATTCAAGTTACTTCTGAT
TGGCGACTCAGGGGTTGGAAAGAATTGCCTTCTTCTTAGGTTTGCAGATG
ATACATATACAGAAAGCTACATCAGCACAATTGGTGTGGATTTCAAAATA
AGAACTATAGAGTTAGACGGGAAAACAATCAAGCTTCAAATATGGGACAC
AGCAGGCCAGGAAAGATTTCGAACAATCACCTCCAGTTATTACAGAGGAG
CCCATGGCATCATAGTTGTGTATGATGTGACAGATCAGGAGTCCTTCAAT
AATGTTAAACAGTGGCTGCAGGAAATAGATCGTTATGCCAGTGAAAATGT
CAACAAATTGTTGGTAGGGAACAAATGTGATCTGACCACAAAGAAAGTAG
TAGACTACACAACAGCGAAGGAATTTGCTGATTCCCTTGGAATTCCGTTT
TTGGAAACCAGTGCTAAGAATGCAACGAATGTAGAACAGTCTTTCATGAC
GATGGCAGCTGAGATTAAAAAGCGAATGGGTCCCGGAGCAACAGCTGGTG
GTGCTGAGAAGTCCAATGTTAAAATTCAGAGCACTCCAGTCAAGCAGTCA
GGTGGAGGTTGCTGCTAA;
or
(SEQ ID NO: 20, MG-008 ORF mRNA Sequence with 5′
Luciferase Tag)
AUGGAAGACGCCAAAAACAUAAAGAAAGGCCCGGCGCCAUUCUAUCCGCU
AGAGGAUGGAACCGCUGGAGAGCAACUGCAUAAGGCUAUGAAGAGAUACG
CCCUGGUUCCUGGAACAAUUGCUUUUACAGAUGCACAUAUCGAGGUGAAC
AUCACGUACGCGGAAUACUUCGAAAUGUCCGUUCGGUUGGCAGAAGCUAU
GAAACGAUAUGGGCUGAAUACAAAUCACAGAAUCGUCGUAUGCAGUGAAA
ACUCUCUUCAAUUCUUUAUGCCGGUGUUGGGCGCGUUAUUUAUCGGAGUU
GCAGUUGCGCCCGCGAACGACAUUUAUAAUGAACGUGAAUUGCUCAACAG
UAUGAACAUUUCGCAGCCUACCGUAGUGUUUGUUUCCAAAAAGGGGUUGC
AAAAAAUUUUGAACGUGCAAAAAAAAUUACCAAUAAUCCAGAAAAUUAUU
AUCAUGGAUUCUAAAACGGAUUACCAGGGAUUUCAGUCGAUGUACACGUU
CGUCACAUCUCAUCUACCUCCCGGUUUUAAUGAAUACGAUUUUGUACCAG
AGUCCUUUGAUCGUGACAAAACAAUUGCACUGAUAAUGAACUCCUCUGGA
UCUACUGGGUUACCUAAGGGUGUGGCCCUUCCGCAUAGAACUGCCUGCGU
CAGAUUCUCGCAUGCCAGAGAUCCUAUUUUUGGCAAUCAAAUCAUUCCGG
AUACUGCGAUUUUAAGUGUUGUUCCAUUCCAUCACGGUUUUGGAAUGUUU
ACUACACUCGGAUAUUUGAUAUGUGGAUUUCGAGUCGUCUUAAUGUAUAG
AUUUGAAGAAGAGCUGUUUUUACGAUCCCUUCAGGAUUACAAAAUUCAAA
GUGCGUUGCUAGUACCAACCCUAUUUUCAUUCUUCGCCAAAAGCACUCUG
AUUGACAAAUACGAUUUAUCUAAUUUACACGAAAUUGCUUCUGGGGGCGC
ACCUCUUUCGAAAGAAGUCGGGGAAGCGGUUGCAAAACGCUUCCAUCUUC
CAGGGAUACGACAAGGAUAUGGGCUCACUGAGACUACAUCAGCUAUUCUG
AUUACACCCGAGGGGGAUGAUAAACCGGGCGCGGUCGGUAAAGUUGUUCC
AUUUUUUGAAGCGAAGGUUGUGGAUCUGGAUACCGGGAAAACGCUGGGCG
UUAAUCAGAGAGGCGAAUUAUGUGUCAGAGGACCUAUGAUUAUGUCCGGU
UAUGUAAACAAUCCGGAAGCGACCAACGCCUUGAUUGACAAGGAUGGAUG
GCUACAUUCUGGAGACAUAGCUUACUGGGACGAAGACGAACACUUCUUCA
UAGUUGACCGCUUGAAGUCUUUAAUUAAAUACAAAGGAUACCAGGUGGCC
CCCGCUGAAUUGGAGUCGAUAUUGUUACAACACCCCAACAUCUUCGACGC
GGGCGUGGCAGGUCUUCCCGACGAUGACGCCGGUGAACUUCCCGCCGCCG
UUGUUGUUUUGGAGCACGGAAAGACGAUGACGGAAAAAGAGAUCGUGGAU
UACGUCGCCAGUCAAGUAACAACCGCCAAAAAGUUGCGCGGAGGAGUUGU
GUUUGUGGACGAAGUACCGAAAGGUCUUACCGGAAAACUCGACGCAAGAA
AAAUCAGAGAGAUCCUCAUAAAGGCCAAGAAGGGCGGAAAGUCCAAAUUG
AUGUCCAGCAUGAAUCCCGAAUAUGAUUAUUUAUUCAAGUUACUUCUGAU
UGGCGACUCAGGGGUUGGAAAGAAUUGCCUUCUUCUUAGGUUUGCAGAUG
AUACAUAUACAGAAAGCUACAUCAGCACAAUUGGUGUGGAUUUCAAAAUA
AGAACUAUAGAGUUAGACGGGAAAACAAUCAAGCUUCAAAUAUGGGACAC
AGCAGGCCAGGAAAGAUUUCGAACAAUCACCUCCAGUUAUUACAGAGGAG
CCCAUGGCAUCAUAGUUGUGUAUGAUGUGACAGAUCAGGAGUCCUUCAAU
AAUGUUAAACAGUGGCUGCAGGAAAUAGAUCGUUAUGCCAGUGAAAAUGU
CAACAAAUUGUUGGUAGGGAACAAAUGUGAUCUGACCACAAAGAAAGUAG
UAGACUACACAACAGCGAAGGAAUUUGCUGAUUCCCUUGGAAUUCCGUUU
UUGGAAACCAGUGCUAAGAAUGCAACGAAUGUAGAACAGUCUUUCAUGAC
GAUGGCAGCUGAGAUUAAAAAGCGAAUGGGUCCCGGAGCAACAGCUGGUG
GUGCUGAGAAGUCCAAUGUUAAAAUUCAGAGCACUCCAGUCAAGCAGUCA
GGUGGAGGUUGCUGCUAA;

or a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity therewith and encoding a Rab1a^{GDP} preferentially binding GDP;
or a nucleic acid sequence equivalent to any of the above sequences due to codon redundancy.

In another embodiment, there is provided herein a polypeptide comprising the amino acid sequence:

(SEQ ID NO: 6; Human Rab1a^{S25N})
MSSMNPEYDYLFKLLLIGDSGVGKNCLLLRFADDTYTESYISTIGVDFKI
RTIELDGKTIKLQIWDTAGQERFRTITSSYYRGAHGIIVVYDVTDQESFN
NVKQWLQEIDRYASENVNKLLVGNKCDLTTKKVVDYTTAKEFADSLGIPF
LETSAKNATNVEQSFMTMAAEIKKRMGPGATAGGAEKSNVKIQSTPVKQS
GGGCC;
(SEQ ID NO: 9; Mouse Rab1^{N124I})
MGDYKDDDDKGGSGGSSMNPEYDYLFKLLLIGDSGVGKSCLLLRFADDTY
TESYISTIGVDFKIRTIELDGKTIKLQIWDTAGQERFRTITSSYYRGAHG
IIVVYDVTDQESFNNVKQWLQEIDRYASENVNKLLVGIKCDLTTKKVVDY
TTAKEFADSLGIPFLETSAKNATNVEQSFMTMAAEIKKRMGPGATAGGAE
KSNVKIQSTPVKQSGGGCC.

the amino acid sequence of human Rab1a^{D41N}; or

(SEQ ID NO: 18; Human Rab1a^{D47N})
MSSMNPEYDYLFKLLLIGDSGVGKSCLLLRFADDTYTESYISTIGVNFKI
RTIELDGKTIKLQIWDTAGQERFRTITSSYYRGAHGIIVVYDVTDQESFN
NVKQWLQEIDRYASENVNKLLVGNKCDLTTKKVVDYTTAKEFADSLGIPF
LETSAKNATNVEQSFMTMAAEIKKRMGPGATAGGAEKSNVKIQSTPVKQS
GGGCC;

or a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity with any of these sequences and preferentially binding GDP;
for use in increasing cell viability of a dopaminergic neuron, or for preventing or treating dopaminergic neuronal death, in a subject in need thereof; or for use in increasing cell viability of a dopaminergic neuron in vitro or in vivo.

In an embodiment of the above polypeptide, the polypeptide is for use in reducing symptoms of Parkinson's disease in a subject, or for preventing or treating Parkinson's disease, in a subject in need thereof.

In an embodiment of the above polypeptide, the symptoms of Parkinson's disease include at least one of the following: static tremor, postural imbalance, bradykinesia, muscle rigidity, punding, dyskinesias, hallucinations, impulse control disorders and sleep disorders.

In another embodiment, there is provided herein a pharmaceutical composition comprising
a GDP-bound form of Rab1a (Rab1a^{GDP}), one or more expressible nucleic acids encoding Rab1a^{GDP}, or a combination thereof; and
another anti-Parkinson agent.

In yet another embodiment, there is provided herein a kit comprising any one or more of:
a GDP-bound form of Rab1a (Rab1a^{GDP});
one or more expressible nucleic acids encoding Rab1a^{GDP};
an anti-Parkinson agent;
instructions for performing any of the method or methods as described herein; or
   any combinations thereof.

As will be understood, in certain embodiments of any of the methods, uses, or polypeptides for use as described herein, the dopaminergic may be any of a wide variety of dopaminergic types. As described in Example 1 below, results from testing indicate that anti-Parkinson effects may be observed for variety of different phenotypes, supporting broad anti-Parkinson applicability.

In still another embodiment of any of the above methods, uses, or polypeptides for use, the dopaminergic neuron is located in the midbrain.

In yet another embodiment of any of the above methods, uses, or polypeptides for use, the the dopaminergic neuron is located in the substantia nigra pars compacta (SNpc) or in the substantia nigra pars reticulata (SNr).

As will be understood, lysosome-mediated microautophagy may refer to a cellular process in which a cellular lipid or protein or glycogen substrate, or a portion thereof, is engulfed and degraded by a lysosome. In this particular regard, lysosome-mediated microautophagy is an integral component of cell's bioenergetic process (i.e. ATP generation) as well as biosynthesis process (i.e. synthesizing new biomass through recycling existing “old” materials).

It will also be understood that references to increasing lysosome-mediated microautophagy may refer to an increase in the rate, extent, capacity, or efficacy of the lysosome-mediated microautophagy process in a cell as compared to baseline levels of the cell, or as compared with a corresponding treated or untreated control cell, or as compared to levels in a reference diseased cell or reference cell having accumulation of a protein, lipid, or glycogen substrate.

It will further be understood that increasing lysosome-mediated microautophagy may include restoring or increasing lysosomal motility or lysosomal bidirectional motility in a cell, and/or may include enhancing, increasing, activating, or otherwise restoring or rescuing lysosomal motility activity in a cell. Such restoration or rescue may result in an increase in autophagy (such as, for example, micro- and/or macro-autophagy) and/or lysosomal degradation capacity. As detailed herein, there are several diseases, conditions, and cellular states in which cellular lysosomal motility may be impaired, reduced, blocked, or suppressed. As lysosome-mediated microautophagy may involve lysosomal motility and kiss-and-run events, microautophagy-enhancing agents may restore or increase lysosomal motility in a cell. Lysosomal motility may play an important role in several cellular functions, including lysosome-mediated microautophagy, lysosome-mediated macroautophagy, lysosomal regeneration, and lysosomal maturation processes. References to restoring lysosomal motility and/or lysosomal bidirectional motility may refer to modulating cellular lysosomal motility/bidirectional motility levels back to those of corresponding normal or healthy control cells having baseline levels of lysosomal motility/bidirectional motility. Such modulation may, in certain embodiments, also modulate degradation capacity (i.e. phagy) levels back to those of normal or healthy cells.

It will be understood that lysosomal association-dissociation events between lysosomes and the lipid or protein or glycogen substrate may refer to events where a lysosome associates with a lipid or protein or glycogen substrate (i.e. a lipid droplet or a protein aggregate, for example), acquires at least a portion of the lipid or protein or glycogen substrate, and then dissociates from the lipid or protein or glycogen substrate. Lysosomal association-dissociation (i.e. the “on” and “off”) events between lysosomes and the lipid or protein or glycogen substrate may be considered as “kiss-and-run”-type events. As part of the association (or “kiss”) event, at least a small piece of substrate (i.e. lipid) may be “grabbed” or engulfed by the lysosome from the substrate (i.e. cytosolic lipid droplets, or CLD, for example). In the case of lipid droplet substrates, this may be achieved through the formation of a fusion pore between lysosome and the CLD. As part of the dissociation (or “run”) event, dissociation of the lysosome from the substrate (i.e. CLD) may occur. An increase in lysosomal association-dissociation events may refer to an increase in the rate, extent, or efficacy of lysosomal association-dissociation events in a cell as compared to baseline levels of a corresponding treated or untreated control cell, for example an identical cell treated under identical conditions but without a microautophagy-modulating agent or with a compound or composition that is known not to affect the process.

It will be understood that in certain embodiments, microautophagy-enhancing agents may be used to correct a microautophagy deficiency in a cell, or a cellular condition in which microautophagy is decreased.

The person of skill in the art having regard to the teachings herein will understand that a microautophagy-enhancing agent may be any suitable agent which increases or facilitates the rate, activity, extent, or efficacy of lysosome-mediated microautophagy in a cell, or that increases lysosomal motility or bidirectional motility. In an embodiment, a suitable microautophagy-enhancing agent may be or comprise a GDP-bound form of Rab1a (Rab1a^{GDP}), one or more expressible nucleic acids encoding Rab1a^{GDP}, De, or a combination thereof.

Ras-related protein Rab-1A (i.e. Rab1a) is a protein that in humans is encoded by the RABIA gene. It may control vesicle transport from the endoplasmic reticulum (ER) to the Golgi compartment and on to the cell surface, and may play an important role in IL-8 and growth hormone secretion. In addition, it may play a role in autophagosome assembly in macroautophagy and cellular defense reactions against pathogenic bacteria when it is in its GTP-bound form. It also may regulate motility of endocytic compartment(s).

As described in detail herein, it has now been identified that lysosome-mediated microautophagy of a target protein, lipid, or glycogen substrate in a cell may be increased by treatment with a microautophagy-enhancing agent comprising a GDP-bound form of Rab1a, such as Rab1a^{S25N} Rab1a^{N124I}, Rab1a^{D41N}, Rab1a^{D47N}, or another dominant negative (DN) GDP-bound form of Rab1a.

It will be understood that the specific amino acid or nucleic acid sequence of a particular gene may vary from species to species. By way of example, the human Rab1a amino acid sequence may have homologs in other species having sequence variation from the human sequence. In some embodiments, although homologous sequences may vary between species, the general effect (for example, the phenotypic effect) of a homolog sequence may be substantially similar to the effect of the wild-type sequence in a given cell or subject.

In particular embodiments, a microautophagy-enhancing agent may be, or comprise, a GDP-bound form of Rab1a (Rab1a^{GDP}) such as, for example, Rab1a^{S25N}, Rab1a^{N124I}, Rab1a^{D41N}, Rab1a^{D47N}, or a functional equivalent thereof, or another dominant negative (DN) GDP-bound form of Rab1a. Sequences for human and/or mouse Rab1aWT and certain GDP-bound forms thereof are shown in FIG. 41. Suitable GDP-bound forms of Rab1a may include any suitable Rab1a variant which is “dominant negative”, or which preferentially binds GDP over GTP. Such Rab1a^{GDP} variants may be identified using techniques known in the art (see, for example, Chan, C.-C. et al. Systematic Discovery of Rab GTPases with Synaptic Functions in Drosophila. Current Biology 21, 1704-1715 (2011); Tabancay, A. P. et al. Identification of dominant negative mutants of Rheb GTPase and their use to implicate the involvement of human Rheb in the activation of p70S6K. J. Biol. Chem. 278, 39921-39930 (2003); and Dumas, J. J., Zhu, Z., Connolly, J. L. & Lambright, D. G. Structural basis of activation and GTP hydrolysis in Rab proteins. Structure 7, 413-s2 (1999), each of which is herein incorporated by reference in its entirety).

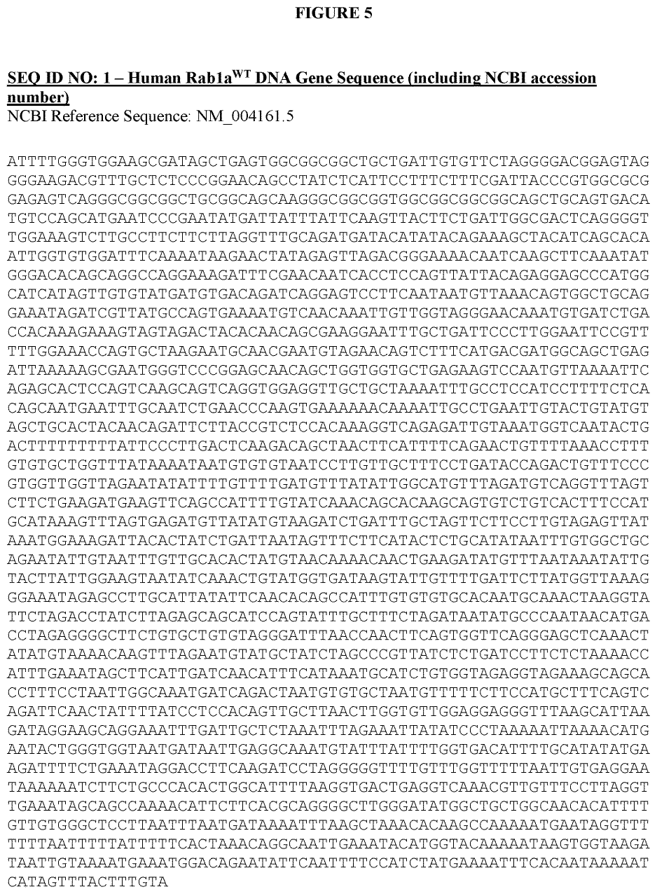

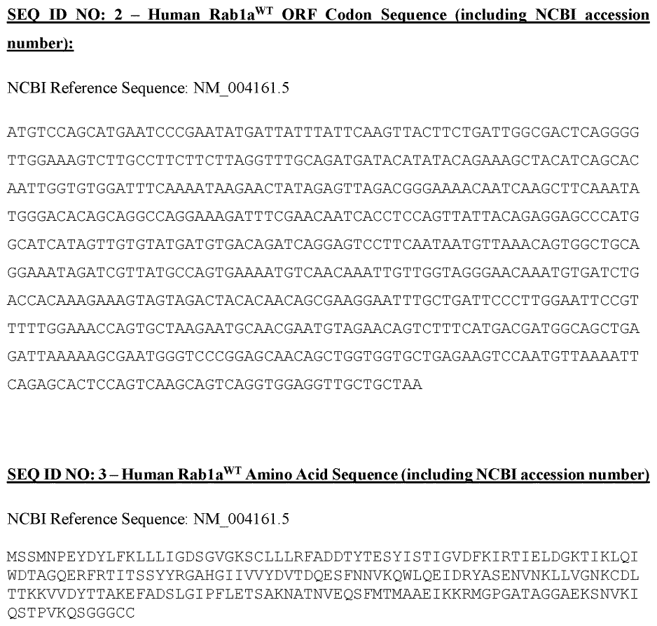

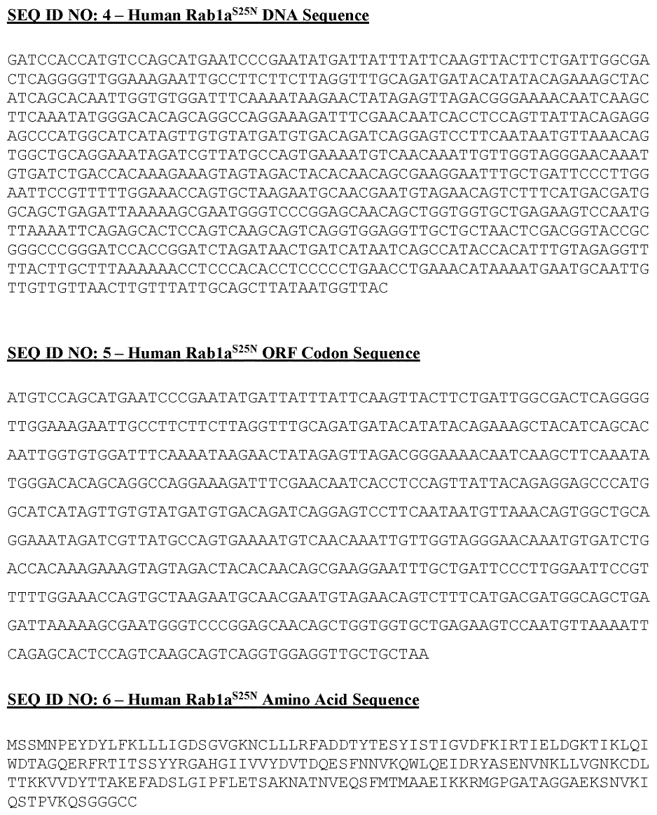

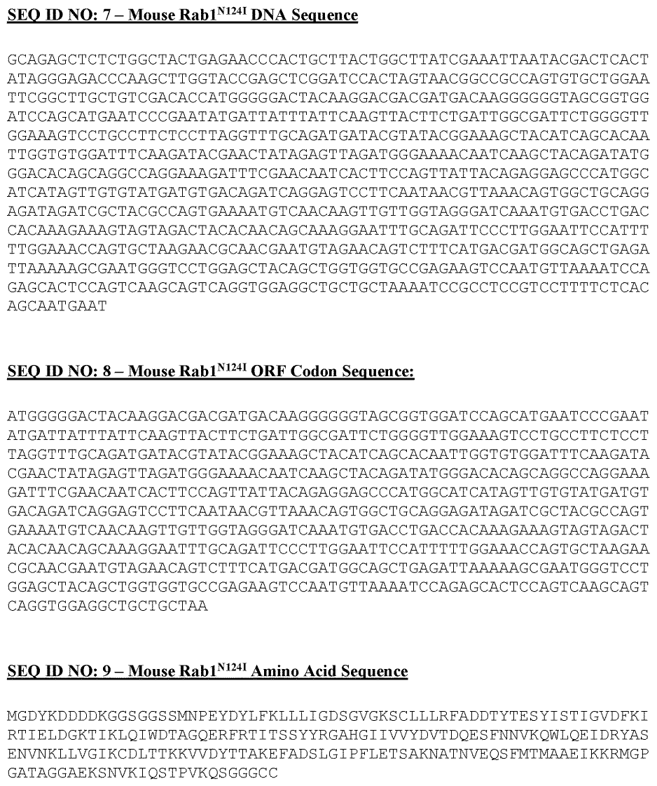

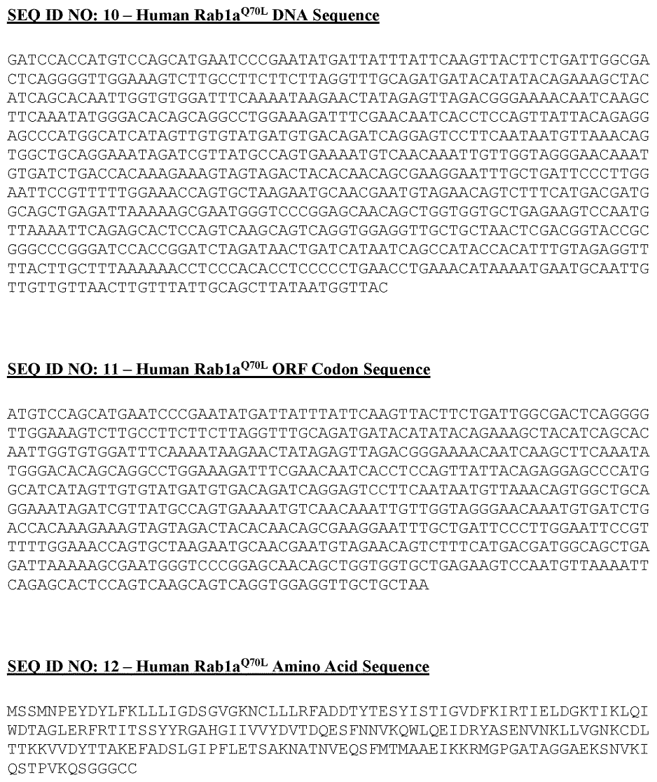

FIG. 5 shows certain sequences of nucleic acids and amino acids/proteins as described herein. In FIG. 5, SEQ ID NOs: 1-3 provide human Rab1a^{WT} DNA gene sequence, ORF codon sequence, and amino acid sequence, respectively; SEQ ID NOs: 4-6 provide human Rab1a^{S25N} DNA gene sequence, ORF codon sequence, and amino acid sequence, respectively; SEQ ID NOs: 7-9 provide mouse Rab1^{N124I} DNA gene sequence, ORF codon sequence, and amino acid sequence, respectively; SEQ ID NOs: 10-12 provide human Rab1a^{Q70L} DNA gene sequence, ORF codon sequence, and amino acid sequence, respectively; SEQ ID NOs: 13-15 provide human Rab1a^{Q63L} DNA gene sequence, ORF codon sequence, and amino acid sequence, respectively; SEQ ID NOs: 16-18 provide human Rab1a^{D47N} DNA gene sequence, ORF codon sequence, and amino acid sequence, respectively; and SEQ ID NOs: 19-21 provide MG-008 ORF DNA sequence with 5′ luciferase tag, MG-008 ORF mRNA sequence with 5′ Luciferase tag, and MG-008 Protein sequence with N-terminal luciferase tag (see Example 1 for further discussion of SEQ ID NOs: 19-21), respectively. Sequence of human Rab1a^{D41N} may be found at https://www.addgene.org/49581/(which is herein incorporated by reference in its entirety), and is commercially available. In certain embodiments, there is provided herein a nucleic acid or amino acid comprising any of these sequences. In certain embodiments, there is provided herein a nucleic acid or amino acid comprising a nucleic acid sequence or amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with any of these sequences (i.e. with any of SEQ ID NOs: 1-21 or the sequence of Rab1a^{D41N}) or an active fragment thereof.

Suitable functional equivalents of Rab1a^{S25N}, Rab1a^{D41N}, Rab1a^{D47N}, and Rab1a^{N124I} may include, for example, suitable Rab1a variants or mutants having at least 80% (or >85%, or >90%, or >95%, or >99%) sequence identity to Rab1a^{WT} or Rab1a^{S25N} or Rab1a^{N124I} or Rab1a^{D41N} or Rab1a^{D47N} and which preferentially bind GDP over GTP while also retaining the relevant cellular/biochemical functions of Rab1a^{S25N} or Rab1a^{N124I} or Rab1a^{D41N} or Rab1a^{D47N} as described in detail herein. In further embodiments, it will be understood that a microautophagy-enhancing agent may be, or comprise, one or more expressible nucleic acids encoding Rab1a^{GDP}, such as any suitable nucleic acid/expression vector (i.e. vector, cassette, mRNA, modified mRNA, plasmid, for example) which encodes for/expresses a GDP-bound form of Rab1a (Rab1a^{GDP}) such as, for example, Rab1a^{S25N} or Rab1a^{N124I} or Rab1a^{D41N} or Rab1a^{D47N}, or a functional equivalent thereof.

As will be understood, sequences are mainly described herein with reference to human and/or mouse homologs. It will be understood that functional equivalents and/or variants may be found in a variety of different species, such as among different mammals. References herein to particular sequence modifications and/or mutants providing for Rab1a^{GDP} (DN) or Rab1a^{GTP} (DA) forms often provide position and modification/mutation information (e.g. S25N, D41N, D47N, N124I; Q70L, Q67L, Q63L) with reference to human and/or mouse homolog/sequence for convenience; however, it will be understood that equivalent DN and/or DA forms may be implemented in homologous sequences from other species and/or other sequences related to the human and/or mouse sequences provided herein, although the positioning and/or nature of the modification/mutation may vary somewhat depending on the particular sequence of interest. By way of example, Rab1^{N124I} is medication/mutation of a mouse sequence. By way of another example, Rab1^{Q67L} is a modification/mutation of a mouse sequence, and there is no Q at the 67^{th} position of human Rab1a; rather, there is a Q in the human sequence at the 63^{rd} position, and so the modification/mutation with reference to the human sequence is Rab1a^{Q63L}.

A person of skill in the art will understand that a microautophagy-reducing agent may be or comprise any one or more of a GTP-bound form of Rab1a (Rab1a^{GTP}), one or more expressible nucleic acids encoding Rab1a^{GTP}, Rab1a wild-type (Rab1a^{WT}), or one or more expressible nucleic acids encoding Rab1a^{WT}. In certain embodiments, a Rab1a^{GTP} may be or comprise Rab1a^{Q70L} Rab1a^{Q67L} (in mouse sequence), Rab1a^{Q63L} (in human Rab1a sequence), or a functional equivalent thereof, or another dominant active (DA) GTP-bound form of Rab1a. GTP-bound forms of Rab1a may include any Rab1a variant which is “dominant active”, or which preferentially binds GTP over GDP. Such Rab1a^{GTP} variants may be identified using techniques known in the art (see, for example, Chan, C.-C. et al. Systematic Discovery of Rab GTPases with Synaptic Functions in Drosophila. Current Biology 21, 1704-1715 (2011); Tabancay, A. P. et al. Identification of dominant negative mutants of Rheb GTPase and their use to implicate the involvement of human Rheb in the activation of p70S6K. J. Biol. Chem. 278, 39921-39930 (2003); and Dumas, J. J., Zhu, Z., Connolly, J. L. & Lambright, D. G. Structural basis of activation and GTP hydrolysis in Rab proteins. Structure 7, 413-s2 (1999), each of which is herein incorporated by reference in its entirety), such as those used to identify Rab1a^{Q70L} and/or Rab1a^{Q67L} and/or Rab1a^{Q63L}.

It will be understood that in certain embodiments, treatment with a microautophagy-enhancing agent, such as Rab1a^{GDP}, may comprise introducing the Rab1a^{GDP} protein to a cell, expressing Rab1a^{GDP} within the cell, or both, for example.

It will be understood that expression of a particular protein within a cell may refer to the production of a polypeptide from a nucleic acid sequence encoding the polypeptide. Gene expression may include both transcription and translation processes, and so gene expression may refer to production of a nucleic acid sequence such as an mRNA (i.e. transcription), production of a protein (i.e. translation), or both. It will further be understood that overexpression of a particular gene in a cell may refer to increasing the expression of a particular gene within a cell as compared to wild-type, baseline, or untreated levels. Overexpression, or introduction of a mutant gene, into cells may be accomplished using any of several methods known in the art. By way of example, a vector (either viral, plasmid, or other) comprising one or more copies of the particular gene each driven by a suitable promoter sequence (for example, a constitutive or inducible promoter), or an mRNA or chemically modified version thereof may be introduced into cells via transfection, electroporation, or viral infection, or another suitable method know in the art. Suitable expression vector techniques for overexpressing or introducing a particular gene into a cell are known in the art (see, for example, Molecular Cloning: A Laboratory Manual (4th Ed.), 2012, Cold Spring Harbor Laboratory Press). The skilled person having regard to the teachings herein will be aware of a wide variety of expressible nucleic acids encoding a particular protein, such as Rab1a^{GDP}, that may be prepared for introduction to a cell (either transiently or long-term via integration into the genome, for example) to provide expression of the protein of interest.

It will be understood that compounds and/or compositions comprising or consisting of one or more of the nucleic acids and/or proteins as described herein may be used. Compositions may additionally comprise one or more pharmaceutically acceptable diluents, carriers, excipients, or buffers. Compositions may be used for administering one or more nucleic acids and/or proteins to a cell in vitro or in vivo.

Introduction of a gene, in the context of inserting a nucleic acid sequence into a cell, may refer to “transfection”, “transformation”, or “transduction”, and may include the incorporation or introduction of a nucleic acid sequence into a eukaryotic cell where the nucleic acid sequence may optionally be incorporated into the genome of the cell, or transiently expressed (for example, transfected mRNA). A protein or enzyme may be introduced into a cell by delivering the protein or enzyme itself into the cell, or by expressing an mRNA encoding the protein or enzyme within the cell, leading to its translation.

As will be known to one of skill in the art, expressible nucleic acids for expressing a particular gene may encode or include features as described in “Genes VII”, Lewin, B. Oxford University Press (2000) or “Molecular Cloning: A Laboratory Manual”, Sambrook et al., Cold Spring Harbor Laboratory, 3rd edition (2001). A nucleotide sequence encoding a polypeptide or protein may be incorporated into a suitable vector, such as a commercially available vector. Vectors may also be individually constructed or modified using standard molecular biology techniques, as outlined, for example, in Sambrook et al. (Cold Spring Harbor Laboratory, 3rd edition (2001)). The person of skill in the art will recognize that a vector may include nucleotide sequences encoding desired elements that may be operably linked to a nucleotide sequence encoding a polypeptide or protein. Such nucleotide sequences encoding desired elements may include transcriptional promoters, transcriptional enhancers, transcriptional terminators, translational initiators, translational terminators, ribosome binding sites, 5′-untranslated region, 3′-untranslated regions, cap structure, poly A tail, and/or an origin of replication. Selection of a suitable vector may depend upon several factors, including, without limitation, the size of the nucleic acid to be incorporated into the vector, the type of transcriptional and translational control elements desired, the level of expression desired, copy number desired, whether chromosomal integration is desired, the type of selection process that is desired, or the host cell or the host range that is intended to be transformed.

The person of skill in the art will understand that biomolecules and/or compounds described herein may be provided in pharmaceutical compositions together with a pharmaceutically acceptable diluent, carrier, or excipient, and/or together with one or more separate active agents or drugs as part of a pharmaceutical combination or pharmaceutical composition. In certain embodiments, the biomolecules, compounds, and/or pharmaceutical compositions may be administered in a treatment regimen simultaneously, sequentially, or in combination with other drugs or pharmaceutical compositions, either separately or as a combined formulation or combination.

Biomolecules, compounds, and/or compositions as described herein may include one or more pharmaceutically acceptable excipients, diluents, and/or carriers. A pharmaceutically acceptable carrier, diluent, or excipient may include any suitable carrier, diluent, or excipient known to the person of skill in the art. Examples of pharmaceutically acceptable excipients may include, but are not limited to, cellulose derivatives, sucrose, and starch. The person of skill in the art will recognize that pharmaceutically acceptable excipients may include suitable fillers, binders, lubricants, buffers, glidants, and disentegrants known in the art (see, for example, Remington: The Science and Practice of Pharmacy (2006)). Examples of pharmaceutically acceptable carriers, diluents, and excipients may be found in, for example, Remington's Pharmaceutical Sciences (2000-20th edition) and in the United States Pharmacopeia: The National Formulary (USP 24 NF19) published in 1999.

It will also be understood that one or more conservative amino acid substitutions may be possible. As will be recognized, a conservative amino acid substitution may include one in which an amino acid is substituted for another amino acid having similar properties such that the folding, activity, or other functionality of the protein is not significantly affected. Examples of aromatic amino acids, which may be substitutable, may include phenylalanine, tryptophan, and tyrosine. Examples of interchangeable hydrophobic amino acids, which may be substitutable, may include leucine, isoleucine, methionine, and valine. Examples of interchangeable polar amino acids, which may be substitutable, may include glutamine and asparagine. Examples of interchangeable basic amino acids, which may be substitutable, may include arginine, lysine, and histidine. Examples of interchangeable acidic amino acids, which may be substitutable, may include aspartic acid and glutamic acid. Finally, examples of interchangeable small amino acids, which may be substitutable, may include alanine, serine, threonine, cysteine, and glycine.

As described in detail herein, dominant-negative (DN) Rab1a (e.g. GDP-bound Rab1a, Rab1a^{GDP}) may be used for increasing microautophagy, which may provide for degradation of a cellular target of interest such as a disease-related protein, glycogen, or lipid. As will be understood, any suitable Rab1a DN or Rab1a^{GDP} may be used. A variety of Rab1a DN proteins will be known to the person of skill in the art having regard to the teachings herein. In certain embodiments, generally any suitable dominant negative form of Rab1a (i.e. a constant/locked Rab1a in its GDP form) may be used to promote lysosomal movement toward the cytosol and peripheral of the cells (from perinuclear region of the cells), and stimulate direct lysosome interaction with target substrates, for example.

In certain embodiments, it is contemplated that expression of Rab1a DN (GDP form) may stimulate activation of mTORC1/mTORC2 and AKT based on their effect on lysosomal positioning and promoting lysosome movement toward the peripheral and toward the target substrates (see also Jia, R. & Bonifacino, J. S. Lysosome Positioning Influences mTORC2 and AKT Signaling. Molecular Cell 75, 26-38.e3 (2019)).

It is contemplated herein that different genetic modifications of Rab1a which lock this protein in its constant GDP-bound state may be used to promote direct lysosome interaction and piecemeal engulfment of different target substrate(s) (such as one or more protein, lipid, and/or glycogen target(s)). Rab1a constant GDP-bound form (DN) is generally not available in normal physiological conditions, in which the native protein Rab1a is constantly shifting between its GTP and GDP forms. It is contemplated that in certain embodiments, genetic mutation and/or amino acid substitution/modification may be used to lock this GTPase in its GDP-bound form (or constant GTP form), and prevent it from going to its GTP state (or GDP state). It is contemplated that in certain embodiments, any suitable modification(s) that can substantially keep the integrity of the GDP form/state of GTPases (which may exert an impact on signaling as well as an effect on lysosome movement to peripheral/cytosol) may promote microautophagy and degradation of target substrate(s) through lysosomal piecemeal engulfment.

In certain embodiments, Rab1a^{GDP} may be administered to a particular cell type or subject in need thereof in generally any suitable manner which may be selected to suit the particular cell type, subject, and/or indication. In certain embodiments, a nucleic acid sequences encoding and capable of expressing the Rab1a^{GDP} may be administered to the subject or introduced to the cell type via any suitable transfection or nucleic acid delivery approach as will be known to the person of skill in the art having regard to the teachings herein. In certain embodiments, delivery may be based on DNA or RNA transfection (for example, using common transfection regent(s) such as lipofectamine (Invitrogen), FuGENE (Rosche), using DNA adenovirus (gene therapy), or using Modified RNA (i.e. stabilized RNA) delivery to body (using, for example, virus or microvesicle, exosome, or ectosome). In certain embodiments, protein may be administered or delivered to cells in need thereof, optionally assisted with any suitable technique or delivery vehicle for facilitating protein delivery to a cell or cells.

In certain embodiments, Rab1a^{GDP} or Rab1a^{DN} may be administered to a particular cell type, or to a patient, using lipid nanoparticles (LNPs). It is envisioned that LNPs would encapsulate Rab1a^{GDP} or Rab1a^{DN}, nucleic acids capable of expressing Rab1a^{GDP} or Rab1a^{DN}, or a combination thereof. LNPs are an advanced non-viral gene delivery system. LNPs allow someone skilled in the art to safely and effectively deliver nucleic acids to cells in vitro or in vivo, which has applications in gene editing, rapid vaccine development, immuno-oncology and treatment of rare genetic and undruggable diseases, without being limiting.

LNPs are being used more frequently in the art due to their advantageous properties, including their controlled and sustained-release properties, low toxicity, biocompatibility with tissues and cells, low immune response, increased deliverable gene size, cell free manufacturing, high nucleic acid encapsulation efficiency, potent transfection, and improved penetration into tissues to deliver therapeutics.

LNP formulations may be a combination of a number of lipids/molecules including, but not limited to, ionizable cationic lipids, neutral lipids, helper lipids, phospholipids, poly (lactic-co-glycolic acid) (PLGA), 1,2-distearoyl-sn-glycero-3-phosphocholine (DSPC), cholesterol, and polyethylene glycol (PEG)-lipid conjugates. The LNPs used may be custom made or purchased from commercially available sources, such as, but not limited to, TriLink BioTechnologies or Precision Nano System.

It is also envisioned that LNPs could be replaced by liposomes or polymer nanoparticles.

### Example 1: Rab1a^{DN} (GDP Form) Reduces Symptoms of Parkinson's Disease, Reduces Dopaminergic Neuronal Cell Death and Stimulates Microautophagy

A series of experiments were performed to investigate the anti-Parkinson effects of Rab1a^{DN} (also referred to here as MG-008) on a wide variety of symptoms of Parkinson's disease and dopaminergic neuronal cell death. Male Sprague-Dawley rats (8 weeks old, n=16) were intracranially injected with 6-OHDA into the medial forebrain bundle (MFB) to generate hemiparkinsonian rat model. Four weeks post-6-OHDA injection, dyskinesia was manifested and half of the rats (n=8) were administered with MG008 (encoded with mRNA and encapsulated with an ANM formulation) through unilateral stereotactic puncture.

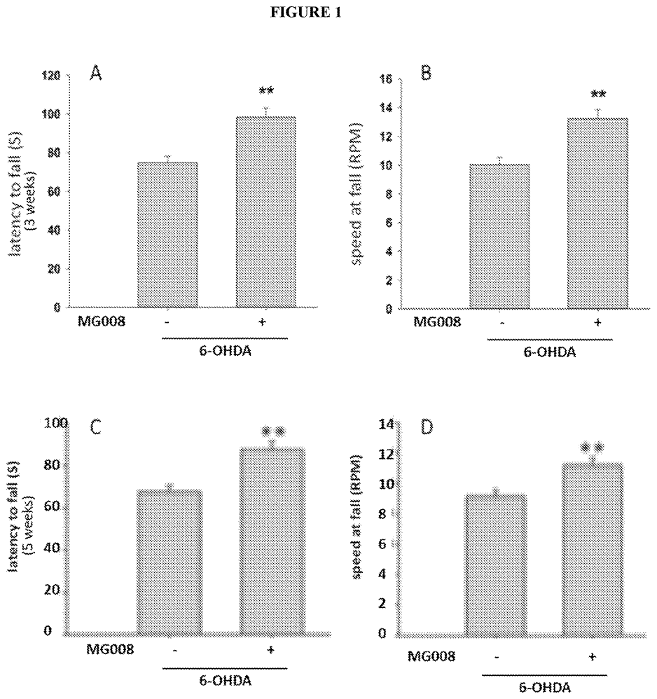

Motor performance was first assessed using the rotarod equipment. Rotarod test was performed at different intervals, as depicted in FIG. 1. Three weeks (panels A and B) and five weeks (panels C and D) post-MG008 treatment, rotarod test was performed with the untreated rats (6-OHDA) and treated rats (+MG008) following a standard operating procedure described previously (https://med.stanford.edu/sbfnl/services/bm/sm/rotor-rod.html). Briefly, rats are placed on a horizontally oriented, rotating cylinder (rod) suspended above a cage floor, which is low enough not to injure the animal, but high enough to induce avoidance of fall. Rats naturally try to stay on the rotating cylinder, or rotarod, and avoid falling to the ground. The length of time that a given rat stays on this rotating rod is a measure of their balance, coordination, physical condition, and motor-planning. The rotarod apparatus was set to accelerate from 4 to 40 rpm in 300 seconds. Trial began when acceleration was started and ended when rats fell off from the rod. The procedure was repeated for total of three trials separated by 15 min intertrial intervals. The latency to fall (in seconds) (panels A and C) and the speed at fall (rpm) (panels B and D) were recorded. Statistic analysis was done with one way ANOVA, **p<0.01, n=8. FIG. 1 shows that MG-008 administration leads to increased latency to fall and increased speed of the rotarod to fall as compared to untreated (6-OHDA) rats. This indicates that MG-008 administration improves motor function in hemiparkinsonian rat models.

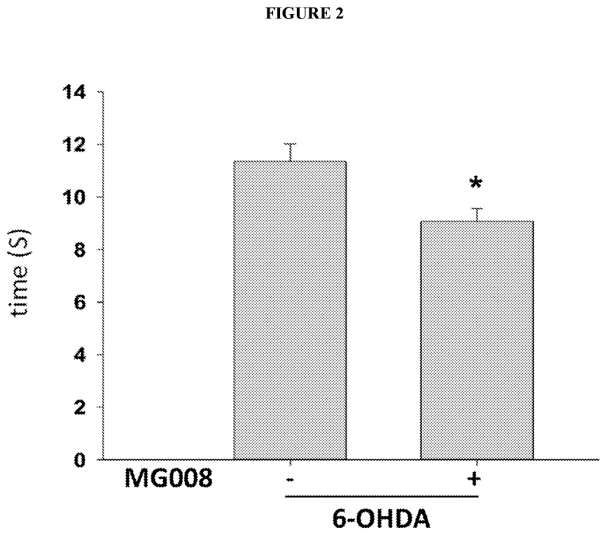

Motor performance was then assessed using beam walking test which assesses motor coordination, particularly of the hindlimb. Rats are placed in one corner of a narrow beam and allowed to walk across the narrow beam from one end to the other for at least three times. The narrow beam measures 1-3 cm wide and is elevated between a pole and their home cage (to attract the rat to the finish point). This training step is useful to achieve a stable baseline measurement. The number of foot slips encountered, and time taken to cross the beam in each trial are recorded. FIG. 2 shows the beam walk test being performed four weeks post-MG008 treatment, to determine the prevalence of akinesia by measuring the time (in seconds) required for the rats to cross the beam. The procedure was repeated for total of three trials separated by 30 second intertrial intervals. Decreased time required to cross the beam is indicative of improved motor performance. Statistic analysis was done with one way ANOVA, *p<0.05, n=8. MG-008 treatment was shown to improve motor coordination in hemiparkinsonian rats, evidenced by the decrease in the amount of time needed to cross the beam as compared to untreated rats (6-OHDA) (FIG. 2).

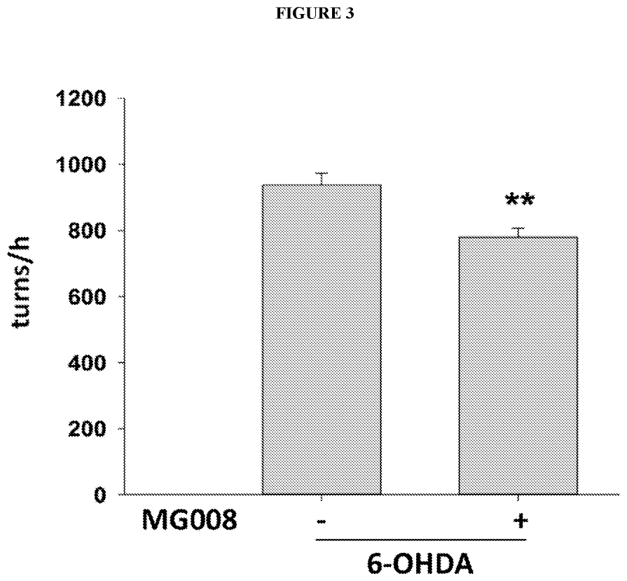

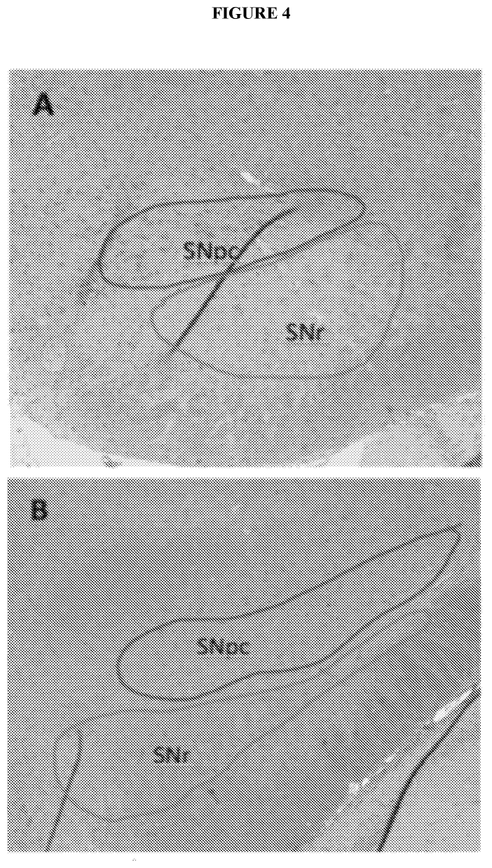

Motor performance was then assessed using the amphetamine rotation test which is commonly used to monitor the extent of motor impairment induced by a lesion such as 6-OHDA injection. This test has become the standard tool to demonstrate functional recovery or the efficacy of neuroprotective interventions aimed to preserve or restore DA neuron function. FIG. 3 shows apomorphine (APO)-induced contralateral rotation test being performed four weeks post-MG008 treatment, to assess the effect of MG008 on the motor performance in the hemiparkinsonian rats with unilateral damage of dopaminergic neurons in the substantia nigra caused by 6-OHDA injections into MFB. Turning rates of rats in a rotometer apparatus, expressed as mean 360°/hour, was recorded automatically over a full test period (typically 60 min). Reduced turning rate is indicative of recovered dopaminergic neurons in rats. Statistic analysis was done with one way ANOVA, **p<0.01, n=8. FIG. 3 shows that MG-008 treatment of hemiparkinsonian rats decreased turning rates and showed improved motor performance, as compared to untreated rats (6-OHDA).

Dopaminergic neuronal cell death was then examined in the hemiparkinsonian rats with unilateral damage of dopaminergic neurons in the substantia nigra caused by 6-OHDA injections into MFB. FIG. 5 shows hematoxylin and eosin staining in the substantia nigra. Eight weeks post-MG008 treatment, the rats were sacrificed, and the brain tissue was retrieved for stereological examination using optical fractionator. Substantia nigra pars compacta (SNpc) and substantia nigra pars reticulata (SNr) regions of MG008 treated (panel A) and control (panel B) rats are shown. In contrast to marked neuron degeneration observed in control rats (6-OHDA, panel B), neurons in MG008-treated rats (panel A) showed normal cytoplasm staining and healthy appearance in cell morphology, indicative of restored dopaminergic neurons in the hemiparkinsonian rats.

One or more illustrative embodiments have been described by way of example. It will be understood to persons skilled in the art that a number of variations and modifications can be made without departing from the scope of the invention as defined in the claims.

### Example 2: Lipid Nanoparticle Encapsulated Rab1a^{DN} (GDP Form) Reduces Symptoms of Parkinson's Disease

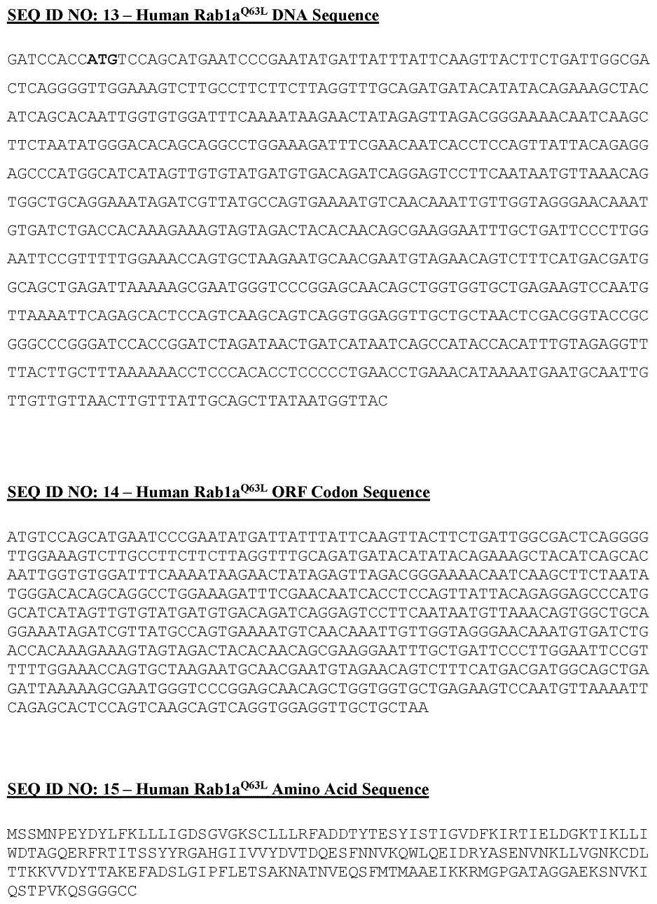

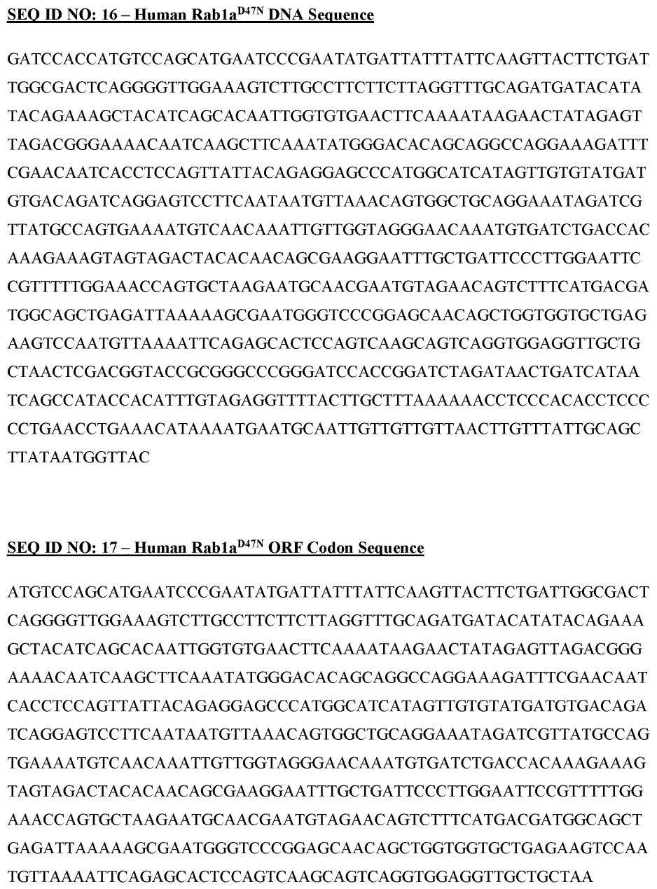

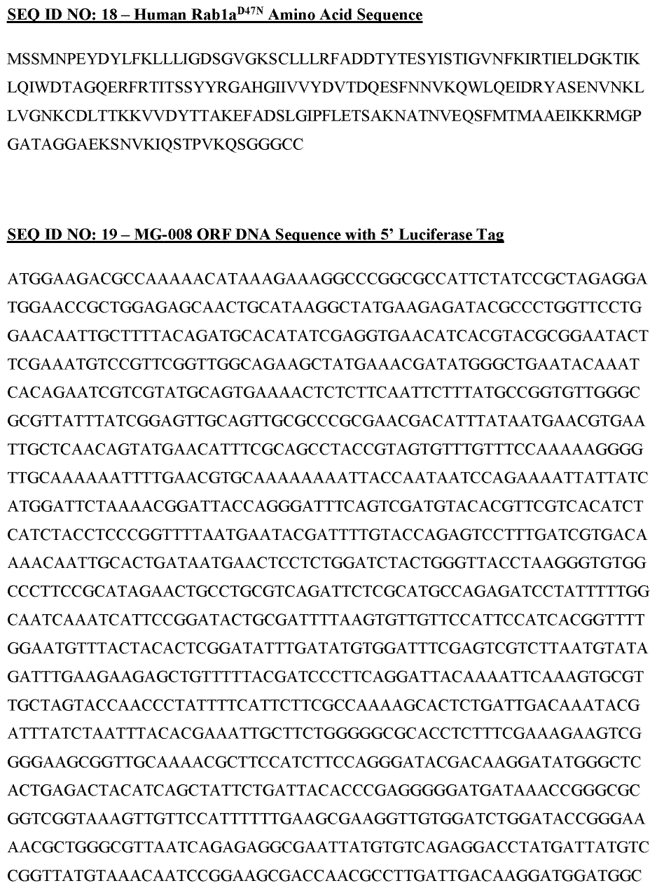

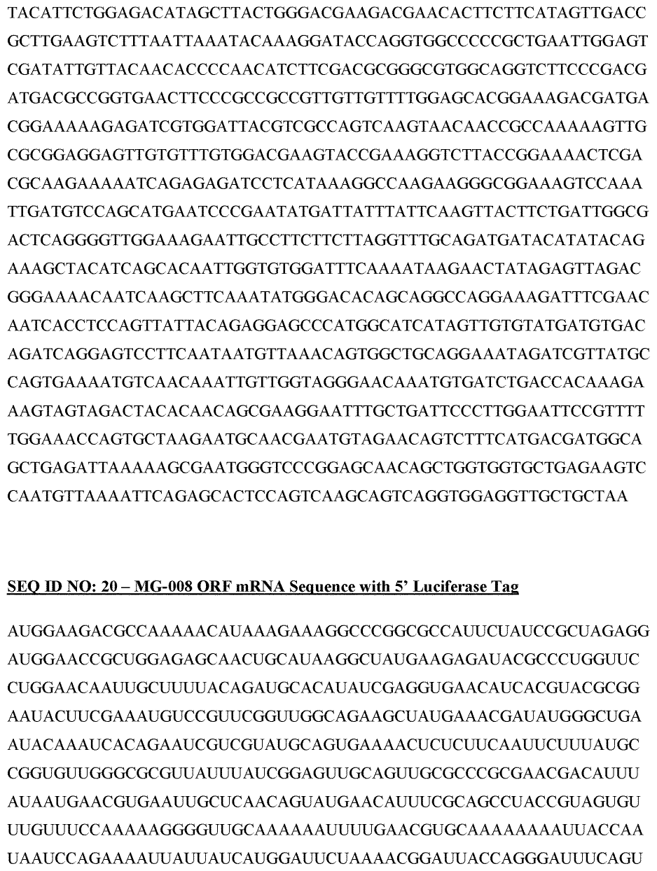

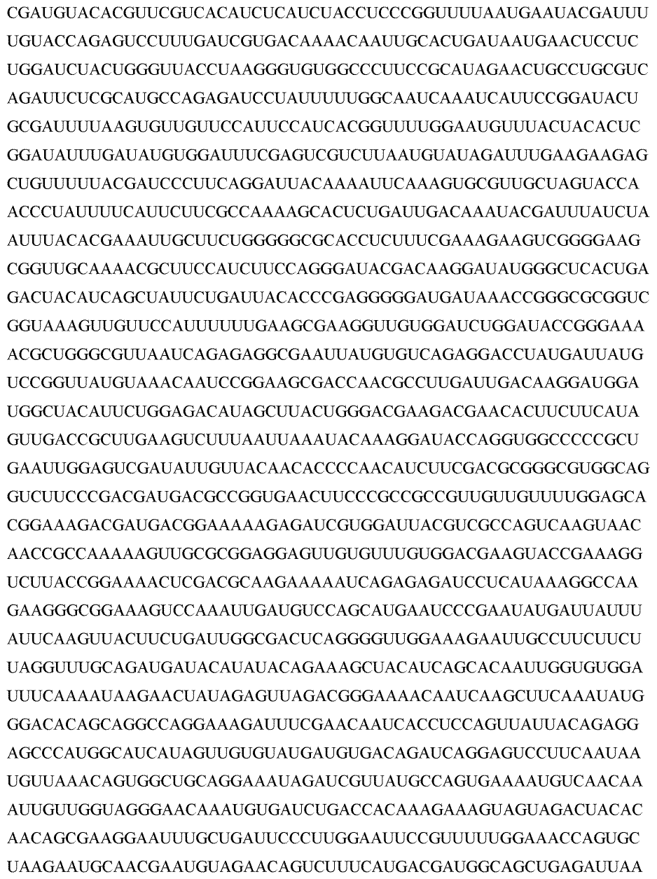

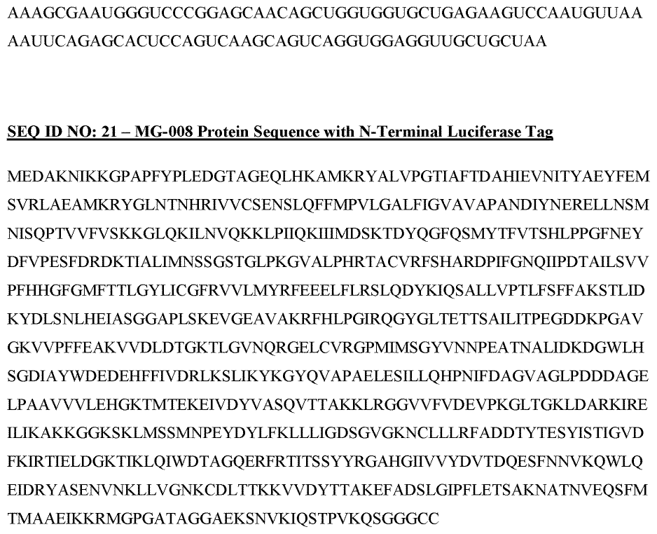

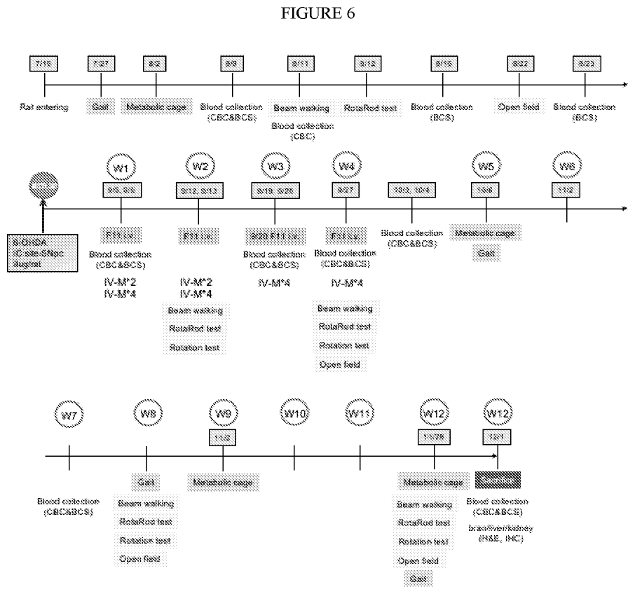

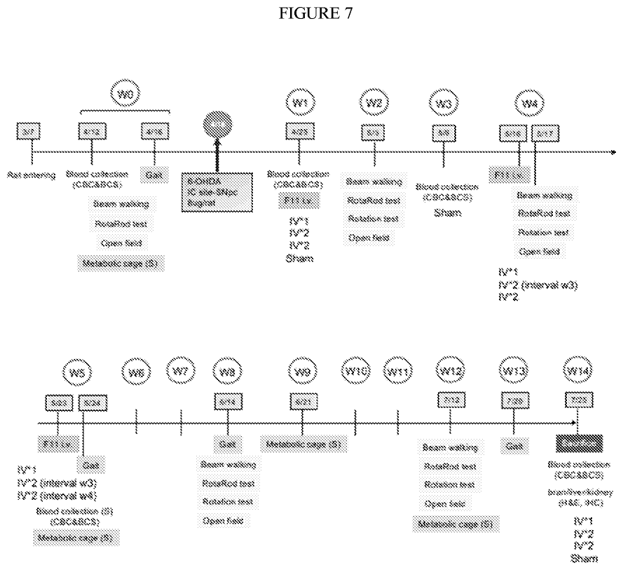

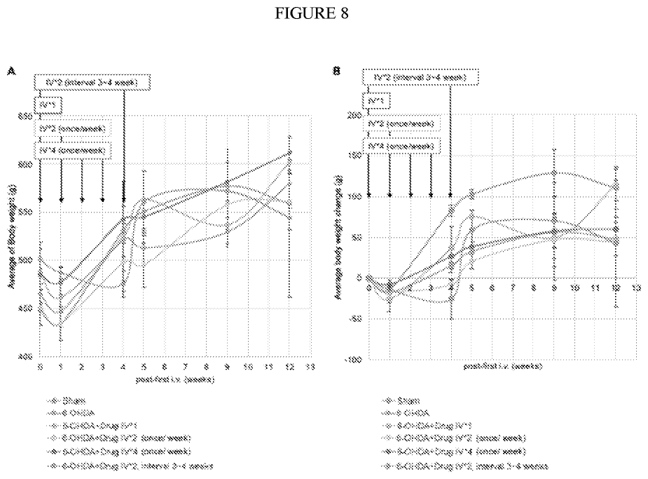

A series of experiments were performed to investigate the effects of F11 treatment on symptoms of Parkinson's disease and dopaminergic neuron cell viability. F11, as mentioned in the following paragraphs and in FIGS. 6 and 7, is Rab1a^{DN} encapsulated in lipid nanoparticles (LNPs).

The two experimental schedules outlined in FIGS. 6 and 7 show the various dosing schedules used for F11 in hemiparkinsonian rat models. Both experimental schedules comprise pre-dosing measurements including gait, blood collection and testing (complete blood count (CBC) and BCS), beam walking, rotarod testing, open field testing, and metabolic cage testing. The principles behind beam walking and rotarod testing were discussed in Example 1. Open field testing is an experimental test used to assess anxiety and general locomotor activity, and is well known by a person of skill in the art. Metabolic cage testing consists of specialized housing units for small animals, such as rodents, that are designed to allow researchers to closely monitor and measure the metabolic rate, food and water intake, and other physiological parameters of the animals. The cages are often used to study the effects of various factors, such as diet, drugs, and environmental conditions, on the metabolism and behavior of the animals. Metabolic cages are well known in the art.

Following collection of the pre-dosing baseline measurements indicated above, Sprague-Dawley rats were injected intracranially in the substantia nigra pars compacta (SNpc) with 8 μg of 6-OHDA per rat. As indicated in Example 1, this established a hemiparkinsonian rat model from which to test F11 efficacy.

One week after 6-OHDA administration, rats were given intravenous injections according to one of the following dosing regimens:

TABLE 1
F11 dosing regimens used in the Experiments outlined in
FIGS. 6 and 7 as well as Example 2.

| Groups | Description |
|---|---|
| Sham | No F11 |
| IV*1 | 1 dose of F11 administered intravenously (i.v.) |
| IV*2 | 2 doses of F11 via i.v. administration; one dose |
| | per week for two weeks |
| IV*4 | 4 doses of F11 via i.v. administration; one dose |
| | per week for 4 weeks |
| IV*2 | 2 doses of F11 via i.v. administration; doses |
| | separated by 3 weeks |
| IV*2 | 2 doses of F11 via i.v. administration; doses |
| | separated by 4 weeks |

Animals were then put through beam walking, rotarod testing, rotation testing, and open field testing at 2, 4, 8, and 12 weeks post-F11 treatment (FIGS. 6 and 7). Beam walking, rotarod testing, and rotation testing are described in Example 1.

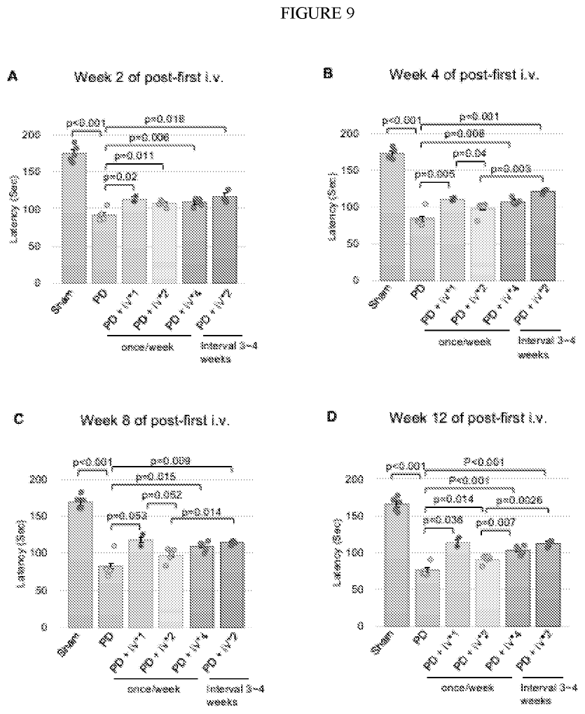

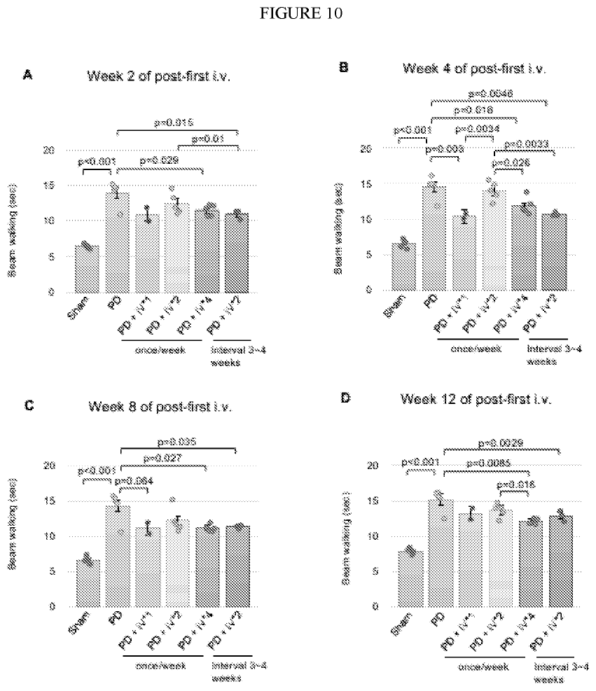

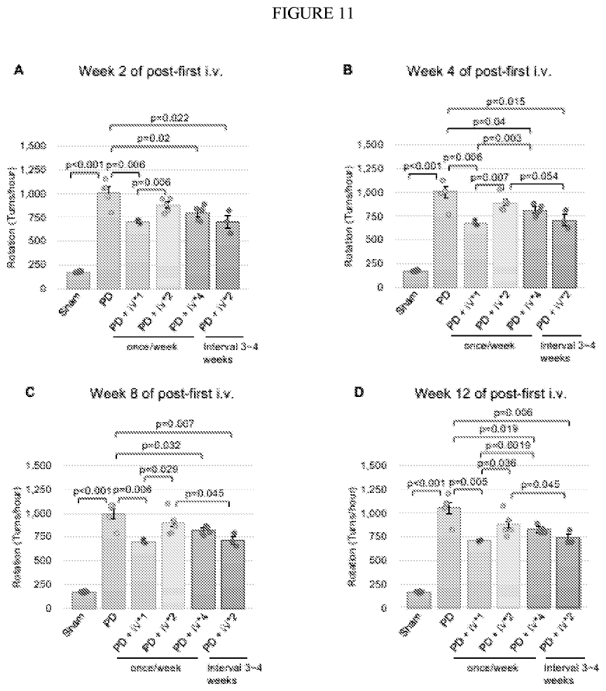

In the rotarod test, the latency to fall increased significantly at 2, 4, 8 and 12 weeks after the first i.v. injection of F11 (FIG. 9) under almost all dosing regimens shown in Table 1, as compared to rats who received 6-OHDA alone. These results show that F11 improved balance and motor coordination in the hemiparkinsonian rats. Similarly, F11-treated hemiparkinsonian rats generally showed improved beam walking (i.e. decreased time taken to cross the beam) 2, 4, 8, and 12 weeks after the initial administration of F11, when compared with 6-OHDA treated rats (FIG. 10). Thus, the beam walking test also shows improved motor coordination in hemiparkinsonian rats following F11 treatment. Lastly, F11-treated hemiparkinsonian rats showed reduced turning rates and improved motor performance on the rotation test, as compared to 6-OHDA treated rats (FIG. 11).

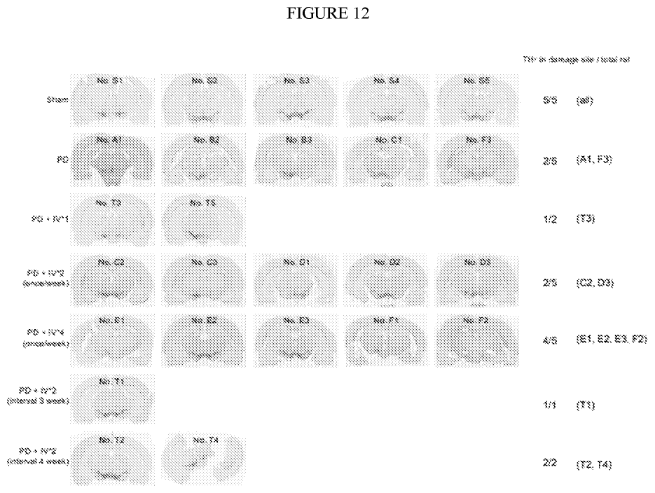

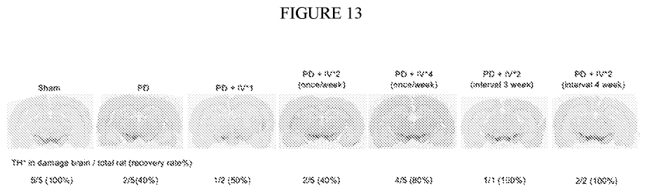

After the 12 weeks of experimentation, the rats were sacrificed and brain, liver and kidney tissues were processed for immunohistochemical and H&E staining. With regard to the brain, tissue slices were stained to look for tyrosine hydroxylase (TH) expression in the substantia nigra of hemiparkinsonian rats with and without F11 treatment. Currently the pathogenesis of PD is not clear, but the condition appears to be related to the degeneration of dopaminergic neurons. With the progress of the disease, neuronal lesions result in the gradual reduction of dopamine synthesis, leading to abnormal discharge in cerebral cortex. TH is a rate-limiting enzyme for dopamine synthesis, which plays an important role in the synthesis of dopamine and may be related to the development of PD. Thus, intervening in the expression and synthesis of TH could improve the neurological symptoms of PD rats. FIGS. 12 and 13 show that intravenous administration of F11 increased TH expression in the substantia nigra in hemiparkinsonian rats, with the most pronounced effects observed in PD+IV*2 (once/week), PD+IV*4 (once/week), PD+IV*2 (interval 3 week), and PD+IV*2 (interval 4 week) groups.

### REFERENCES

Zeng X-S, Geng W-S, Jia J-J, Chen L and Zhang P-P (2018) Cellular and Molecular Basis of Neurodegeneration in Parkinson Disease. Front. Aging Neurosci. 10:109
Schapira, A H (2008). Mitochondria in the aetiology and pathogenesis of Parkinson's disease. Lancet Neurol. 7, 97-109.
Schapira A H & Tolosa E (2010) Molecular and clinical prodrome of Parkinson disease: implications for treatment. Nat Rev Neurol. 2010 June; 6 (6): 309-17
Stoker T B, Torsney K M and Barker R A (2018) Emerging Treatment Approaches for Parkinson's Disease. Front. Neurosci. October; 12 (693)
Li, W.-W., Li, J. & Bao, J.-K. Microautophagy: lesser-known self-eating. Cell. Mol. Life Sci. 69, 1125-1136 (2011).
Fujiwara, Y. et al. Direct uptake and degradation of DNA by lysosomes.—PubMed—NCBI. Autophagy 9, 1167-1171 (2014).
Andrews, N. W. Lysosomes and the plasma membrane. J. Cell Biol. 158, 389-394 (2002).
Hofmann, I. & Munro, S. An N-terminally acetylated Arf-like GTPase is localised to lysosomes and affects their motility. J. Cell. Sci. 119, 1494-1503 (2006).
Fraldi, A. et al. Lysosomal fusion and SNARE function are impaired by cholesterol accumulation in lysosomal storage disorders. The EMBO Journal 29, 3607-3620 (2010).
Pankiv, S. et al. FYCO1 is a Rab7 effector that binds to LC3 and PI3P to mediate microtubule plus end-directed vesicle transport. J. Cell Biol. 188, 253-269 (2010).
R. & Bonifacino, J. S. Lysosome Positioning Influences mTORC2 and AKT Signaling. Molecular Cell 75, 26-38.e3 (2019).
Pous, C. & Codogno, P. Lysosome positioning coordinates mTORC1 activity and autophagy. Nature Cell Biology 13, 342-344 (2011).
Cabukusta, B. & Neefjes, J. Mechanisms of lysosomal positioning and movement. Traffic 19, 761-769 (2018).
Jia, R. & Bonifacino, J. S. Lysosome Positioning Influences mTORC2 and AKT Signaling. Molecular Cell 75, 26-38.e3 (2019)
Pu, J., Guardia, C. M., Keren-Kaplan, T. & Bonifacino, J. S. Mechanisms and functions of lysosome positioning. J. Cell. Sci. 129, 4329-4339 (2016)
Katherine R Parzych, D. J. K. An Overview of Autophagy: Morphology, Mechanism, and Regulation. Antioxid. Redox Signal. 20, 460-473 (2014)
Rabanal-Ruiz, Y. & Korolchuk, V. I. mTORC1 and Nutrient Homeostasis: The Central Role of the Lysosome. Int J Mol Sci 19, 818 (2018)
Jia, R. & Bonifacino, J. S. Lysosome Positioning Influences mTORC2 and AKT Signaling. Molecular Cell 75, 26-38.e3 (2019)
Allyson L Anding, E. H. B. Cleaning House: Selective Autophagy of Organelles. Developmental Cell 41, 10-22 (2017)
De Bortoli, M. et al. Lipid accumulation in human breast cancer cells injured by iron depletors. undefined 37, 1 (2018)
Ipsen, D. H., Lykkesfeldt, J. & Tveden-Nyborg, P. Molecular mechanisms of hepatic lipid accumulation in non-alcoholic fatty liver disease. Cellular and Molecular Life Sciences 75, 3313-3327 (2018)
Nakagawa, H. et al. Lipid Metabolic Reprogramming in Hepatocellular Carcinoma. Cancers 10, 447 (2018)
Namba, Y. et al. Depletion of Lipid Efflux Pump ABCG1 Triggers the Intracellular Accumulation of Extracellular Vesicles and Reduces Aggregation and Tumorigenesis of Metastatic Cancer Cells. Front. Oncol. 8, e0191109 (2018)
Shyu, P., Wong, X. F. A., Crasta, K. & Thibault, G. Dropping in on lipid droplets: insights into cellular stress and cancer. Biosci. Rep. 38, BSR20180764 (2018)
Christos E Zois, A. L. H. Glycogen metabolism has a key role in the cancer microenvironment and provides new targets for cancer therapy. J. Mol. Med. 94, 137-154 (2016)
Zois, C. E., Favaro, E. & Harris, A. L. Glycogen metabolism in cancer. Biochemical Pharmacology 92, 3-11 (2014)
Parachoniak C A, Park M. Dynamics of receptor trafficking in tumorigenicity. Trends Cell Biol. 2012 May; 22 (5): 231-40
Anderson R G, Ghiraldeli L P, Pardee T S. Mitochondria in cancer metabolism, an or-ganelle whose time has come? Biochim Biophys Acta Rev Cancer. 2018 August; 1870 (1): 96-102
Davidson S M, Vander Heiden M G. Critical Functions of the Lysosome in Cancer Bi-ology. Annu Rev Pharmacol Toxicol. 2017 Jan. 6; 57:481-507
Zhitomirsky B, Assaraf Y G. Lysosomes as mediators of drug resistance in cancer. Drug Resist Updat. 2016 January; 24:23-33
Rudzińska M, Parodi A, Soond S M, Vinarov A Z, Korolev D O, Morozov A O, Daglioglu C, Tutar Y, Zamyatnin A A Jr. The Role of Cysteine Cathepsins in Cancer Progression and Drug Resistance. Int J Mol Sci. 2019 Jul. 23; 20 (14): 3602
Kim K H, Lee M S. Autophagy—a key player in cellular and body metabolism. Nat Rev Endocrinol. 2014 June; 10 (6): 322-37
Parzych K R, Klionsky D J. An overview of autophagy: morphology, mechanism, and regulation. Antioxid Redox Signal. 2014 Jan. 20; 20 (3): 460-73
Gong C, Bauvy C, Tonelli G, Yue W, Deloménie C, Nicolas V, Zhu Y, Domergue V, Marin-Esteban V, Tharinger H, Delbos L, Gary-Gouy H, Morel A P, Ghavami S, Song E, Codogno P, Mehrpour M. Beclin 1 and autophagy are required for the tumorigen-icity of breast cancer stem-like/progenitor cells. Oncogene. 2013 May 2; 32 (18): 2261-72, 2272e.1-11
Mathiassen S G, De Zio D, Cecconi F. Autophagy and the Cell Cycle: A Complex Landscape. Front Oncol. 2017 Mar. 31; 7:51
Simon H U, Friis R. ATG5: a distinct role in the nucleus. Autophagy. 2014 January; 10 (1): 176-7
Gozuacik D, Kimchi A. Autophagy as a cell death and tumor suppressor mechanism. Oncogene. 2004 Apr. 12; 23 (16): 2891-906
Pathania A S, Guru S K, Kumar S, Kumar A, Ahmad M, Bhushan S, Sharma P R, Ma-hajan P, Shah B A, Sharma S, Nargotra A, Vishwakarma R, Korkaya H, Malik F. In-terplay between cell cycle and autophagy induced by boswellic acid analog. Sci Rep. 2016 Sep. 29; 6:33146
Fuchs Y, Steller H. Programmed cell death in animal development and disease. Cell. 2011 Nov. 11; 147 (4): 742-58
Kroemer G, Levine B. Autophagic cell death: the story of a misnomer. Nat Rev Mol Cell Biol. 2008 December; 9 (12): 1004-10
Rosebeck S, Alonge M M, Kandarpa M, Mayampurath A, Volchenboum S L, Jasielec J, Dytfeld D, Maxwell S P, Kraftson S J, McCauley D, Shacham S, Kauffman M, Jakubowiak A J. Synergistic Myeloma Cell Death via Novel Intracellular Activation of Caspase-10-Dependent Apoptosis by Carfilzomib and Selinexor. Mol Cancer Ther. 2016 January; 15 (1): 60-71
Scherz-Shouval R, Weidberg H, Gonen C, Wilder S, Elazar Z, Oren M. p53-dependent regulation of autophagy protein LC3 supports cancer cell survival under prolonged starvation. Proc Natl Acad Sci USA. 2010 Oct. 26; 107 (43): 18511-6
Filomeni G, De Zio D, Cecconi F. Oxidative stress and autophagy: the clash between damage and metabolic needs. Cell Death Differ. 2015 March; 22 (3): 377-88
Liou G Y, Storz P. Reactive oxygen species in cancer. Free Radic Res. 2010 May; 44 (5): 479-96
Auten R L, Davis J M. Oxygen toxicity and reactive oxygen species: the devil is in the details. Pediatr Res. 2009 August; 66 (2): 121-7
Perillo B, Di Donato M, Pezone A, Di Zazzo E, Giovannelli P, Galasso G, Castoria G, Migliaccio A. ROS in cancer therapy: the bright side of the moon. Exp Mol Med. 2020 February; 52 (2): 192-203
Martinez-Lopez N, Athonvarangkul D, Singh R. Autophagy and aging. Adv Exp Med Biol. 2015; 847:73-87
Tan M H, Mester J L, Ngeow J, Rybicki L A, Orloff M S, Eng C. Lifetime cancer risks in individuals with germline PTEN mutations. Clin Cancer Res. 2012 Jan. 15; 18 (2): 400-7
Liu E Y, Xu N, O'Prey J, Lao L Y, Joshi S, Long J S, O'Prey M, Croft D R, Beaumatin F, Baudot A D, Mrschtik M, Rosenfeldt M, Zhang Y, Gillespie D A, Ryan K M. Loss of autophagy causes a synthetic lethal deficiency in DNA repair. Proc Natl Acad Sci USA. 2015 Jan. 20; 112 (3): 773-8.
Gan-Or, Z., Dion, P. A., and Rouleau, G. A. (2015). Genetic perspective on the role of the autophagy-lysosome pathway in Parkinson disease. Autophagy 11, 1443-1457.
Polymeropoulos M H, Lavedan, C., Leroy, E., Ide, S. E., Dehejia, A., Dutra, A., Pike, B., Root, H., Rubenstein, J., Boyer, R., et al. (1997). Mutation in the alpha-synuclein gene identified in families with Parkinson's disease. Science 276, 2045-2047
Pupyshev, A. B., Korolenko, T. A., Akopyan, A. A., Amstislavskaya, T. G., and Tikhonova, M. A. (2017). Suppression of autophagy in the brain of transgenic mice with overexpression of capital A, Cyrillic53capital T E, Cyrillic-mutant alpha-synuclein as an early event at synucleinopathy progression. Neurosci. Lett. 672, 140-142.
Smith, W. W., Jiang, H., Pei, Z., Tanaka, Y., Morita, H., Sawa, A., et al. (2005). Endoplasmic reticulum stress and mitochondrial cell death pathways mediate A53T mutant alpha-synuclein-induced toxicity. Hum. Mol. Genet. 14, 3801-3811.
Smith, W. W., Pei, Z., Jiang, H., Moore, D. J., Liang, Y., West, A. B., et al. (2005). Leucine-rich repeat kinase 2 (LRRK2) interacts with parkin, and mutant LRRK2 induces neuronal degeneration. Proc. Natl. Acad. Sci. U.S.A. 102, 18676-18681.
Zharikov, A. D., Cannon, J. R., Tapias, V., Bai, Q., Horowitz, M. P., Shah, V., et al. (2015). shRNA targeting alpha-synuclein prevents neurodegeneration in a Parkinson's disease model. J. Clin. Invest. 125, 2721-2735.
Wang, K., Huang, J., Xie, W., Huang, L., Zhong, C., and Chen, Z. (2016). Beclin1 and HMGB1 ameliorate the alpha-synuclein-mediated autophagy inhibition in PC12 cells. Diagn. Pathol. 11:15.
Zimprich, A., Biskup, S., Leitner, P., Lichtner, P., Farrer, M., Lincoln, S., et al. (2004). Mutations in LRRK2 cause autosomal-dominant parkinsonism with pleomorphic pathology. Neuron 44, 601-607.
Ferreira, M., and Massano, J. (2017). An updated review of Parkinson's disease genetics and clinicopathological correlations. Acta Neurol. Scand. 135, 273-284.
Steger, M., Diez, F., Dhekne, H. S., Lis, P., Nirujogi, R. S., Karayel, O., et al. (2017). Systematic proteomic analysis of LRRK2-mediated Rab GTPase phosphorylation establishes a connection to ciliogenesis. Elife 6: e31012
Pan, P. Y., Li, X., Wang, J., Powell, J., Wang, Q., Zhang, Y., et al. (2017). Parkinson's disease-associated LRRK2 hyperactive kinase mutant disrupts synaptic vesicle trafficking in ventral midbrain neurons. J. Neurosci. 37, 11366-11376
Volpicelli-Daley, L. A., Abdelmotilib, H., Liu, Z., Stoyka, L., Daher, J. P., Milnerwood, A. J., et al. (2016). G2019S-LRRK2 expression ments alphasynuclein sequestration into inclusions in neurons. J. Neurosci. 36, 7415-7427.
Yoon, J. H., Mo, J. S., Kim, M. Y., Ann, E. J., Ahn, J. S., Jo, E. H., et al. (2017). LRRK2 functions as a scaffolding kinase of ASK1-mediated neuronal cell death. Biochim. Biophys. Acta 1864, 2356-2368.
Su, Y. C., Guo, X., and Qi, X. (2015). Threonine 56 phosphorylation of Bcl-2 is required for LRRK2 G2019S-induced mitochondrial depolarization and autophagy. Biochim. Biophys. Acta 1852, 12-21.
Narendra, D., Tanaka, A., Suen, D. F., and Youle, R. J. (2009). Parkin-induced mitophagy in the pathogenesis of Parkinson disease. Autophagy 5, 706-708.
Narendra, D. P., Jin, S. M., Tanaka, A., Suen, D. F., Gautier, C. A., Shen, J., et al. (2010). PINK1 is selectively stabilized on impaired mitochondria to activate Parkin. PLOS Biol.
Tang, F. L., Erion, J. R., Tian, Y., Liu, W., Yin, D. M., Ye, J., et al. (2015a). VPS35 in dopamine neurons is required for endosome-to-golgi retrieval of Lamp2a, a receptor of chaperone-mediated autophagy that is critical for α-synuclein degradation and prevention of pathogenesis of Parkinson's disease. J. Neurosci. 35, 10613-10628
Liu, J., Wang, X., Lu, Y., Duan, C., Gao, G., Lu, L., et al. (2017). Pink1 interacts with alpha-synuclein and abrogates alpha-synuclein-induced neurotoxicity by activating autophagy. Cell Death Dis. 8: e3056.
Cai, Y., Arikkath, J., Yang, L., Guo, M. L., Periyasamy, P., and Buch, S. (2016). Interplay of endoplasmic reticulum stress and autophagy in neurodegenerative disorders. Autophagy 12, 225-244
Xilouri, M., Brekk, O. R., Polissidis, A., Chrysanthou-Piterou, M., Kloukina, I., and Stefanis, L. (2016). Impairment of chaperone-mediated autophagy induces dopaminergic neurodegeneration in rats. Autophagy 12, 2230-2247.
https://med.stanford.edu/sbfnl/services/bm/sm/rotor-rod.html
WO2017/008141—Lysosomal Degradation of Lipids and Proteins and Method of Use Thereof

All references cited herein and throughout this specification are herein incorporated by reference in their entireties.

1. A method for reducing symptoms of Parkinson's disease, or for preventing or treating Parkinson's disease, in a subject in need thereof, or for increasing cell viability of a dopaminergic neuron in vitro or in vivo, said method comprising:
treating the subject or the dopaminergic neuron with a GDP-bound form of Rab1a (Rab1a^{GDP}), one or more expressible nucleic acids encoding Rab1a^{GDP}, or a combination thereof;
thereby increasing cellular levels of Rab1a^{GDP} in the subject or the dopaminergic neuron, resulting in decreased symptoms of Parkinson's disease in the subject or increased cell viability of the dopaminergic neuron.

2. The method of claim 1, wherein the symptoms include at least one of the following: static tremors, postural imbalance, bradykinesia, muscle rigidity, punding, dyskinesias, hallucinations, impulse control disorders and sleep disorders.

3. (canceled)

4. The method of claim 1, wherein the Rab1a^{GDP} is or comprises Rab1a^{S25N}, Rab1a^{N124I}, Rab1a^{D41N}, Rab1a^{D47N}, or another dominant negative (DN) GDP-bound form of Rab1a.

5. The method of claim 1, wherein the Rab1a^{GDP} comprises or consists of the amino acid sequence:
(SEQ ID NO: 6; Human Rab1a^{S25N})
MSSMNPEYDYLFKLLLIGDSGVGKNCLLLRFADDTYTESYISTIGVDFKI
RTIELDGKTIKLQIWDTAGQERFRTITSSYYRGAHGIIVVYDVTDQESFN
NVKQWLQEIDRYASENVNKLLVGNKCDLTTKKVVDYTTAKEFADSLGIPF
LETSAKNATNVEQSFMTMAAEIKKRMGPGATAGGAEKSNVKIQSTPVKQS
GGGCC;
(SEQ ID NO: 9; Mouse Rab1^{N124I})
MGDYKDDDDKGGSGGSSMNPEYDYLFKLLLIGDSGVGKSCLLLRFADDTY
TESYISTIGVDFKIRTIELDGKTIKLQIWDTAGQERFRTITSSYYRGAHG
IIVVYDVTDQESENNVKQWLQEIDRYASENVNKLLVGIKCDLTTKKVVDY
TTAKEFADSLGIPFLETSAKNATNVEQSFMTMAAEIKKRMGPGATAGGAE
KSNVKIQSTPVKQSGGGCC;
the amino acid sequence of human Rab1a^{D41N}; or
(SEQ ID NO: 18; Human Rab1a^{D47N})
MSSMNPEYDYLFKLLLIGDSGVGKSCLLLRFADDTYTESYISTIGVNFKI
RTIELDGKTIKLQIWDTAGQERFRTITSSYYRGAHGIIVVYDVTDQESFN
NVKQWLQEIDRYASENVNKLLVGNKCDLTTKKVVDYTTAKEFADSLGIPF
LETSAKNATNVEQSFMTMAAEIKKRMGPGATAGGAEKSNVKIQSTPVKQS
GGGCC;
or a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity with any of these sequences and preferentially binding GDP.

6.-7. (canceled)

8. The method of claim 1, wherein the Rab1a^{GDP} is in the form of a fusion protein, wherein the Rab1a^{GDP} is fused or otherwise directly or indirectly linked, optionally via a linker, with a signalling or targeting peptide, a fluorescent peptide or other marker or tracer, or another peptide or non-peptide moiety for targeted delivery, facilitating cell uptake, increasing stability or in vivo half-life, or improving another therapeutic, diagnostic, or in vivo property of the Rab1a^{GDP}.

9. The method of claim 8, wherein the fusion protein comprises the amino acid sequence:
(SEQ ID NO: 6; Human Rab1a^{S25N})
MSSMNPEYDYLFKLLLIGDSGVGKNCLLLRFADDTYTESYISTIGVDFKI
RTIELDGKTIKLQIWDTAGQERFRTITSSYYRGAHGIIVVYDVTDQESFN
NVKQWLQEIDRYASENVNKLLVGNKCDLTTKKVVDYTTAKEFADSLGIPF
LETSAKNATNVEQSFMTMAAEIKKRMGPGATAGGAEKSNVKIQSTPVKQS
GGGCC;
or a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity therewith and preferentially binding GDP.

10. The method of claim 8, wherein the fusion protein comprises the amino acid sequence:
(SEQ ID NO: 21)
MEDAKNIKKGPAPFYPLEDGTAGEQLHKAMKRYALVPGTIAFTDAHIEVN
ITYAEYFEMSVRLAEAMKRYGLNTNHRIVVCSENSLQFFMPVLGALFIGV
AVAPANDIYNERELLNSMNISQPTVVFVSKKGLQKILNVQKKLPIIQKII
IMDSKTDYQGFQSMYTFVTSHLPPGFNEYDFVPESFDRDKTIALIMNSSG
STGLPKGVALPHRTACVRFSHARDPIFGNQIIPDTAILSVVPFHHGFGMF
TTLGYLICGFRVVLMYRFEEELFLRSLQDYKIQSALLVPTLFSFFAKSTL
IDKYDLSNLHEIASGGAPLSKEVGEAVAKRFHLPGIRQGYGLTETTSAIL
ITPEGDDKPGAVGKVVPFFEAKVVDLDTGKTLGVNQRGELCVRGPMIMSG
YVNNPEATNALIDKDGWLHSGDIAYWDEDEHFFIVDRLKSLIKYKGYQVA
PAELESILLQHPNIFDAGVAGLPDDDAGELPAAVVVLEHGKTMTEKEIVD
YVASQVTTAKKLRGGVVFVDEVPKGLTGKLDARKIREILIKAKKGGKSKL
MSSMNPEYDYLFKLLLIGDSGVGKNCLLLRFADDTYTESYISTIGVDFKI
RTIELDGKTIKLQIWDTAGQERFRTITSSYYRGAHGIIVVYDVTDQESFN
NVKQWLQEIDRYASENVNKLLVGNKCDLTTKKVVDYTTAKEFADSLGIPF
LETSAKNATNVEQSFMTMAAEIKKRMGPGATAGGAEKSNVKIQSTPVKQS
GGGCC;
or a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity therewith and preferentially binding GDP.

11. (canceled)

12. The method of claim 1, wherein the one or more expressible nucleic acids are DNA-based, or RNA-based.

13. The method of claim 1, wherein the one or more expressible nucleic acids transiently express the Rab1a^{GDP} in the cell, or wherein the one or more expressible nucleic acids integrate in the cell genome and express the Rab1a^{GDP} in the cell.

14. The method of claim 1, wherein the one or more expressible nucleic acids comprise one or more expression vectors, plasmids, or mRNAs encoding and capable of expressing the Rab1a^{GDP} inside the cell.

15. The method of claim 14, wherein the one or more expressible nucleic acids comprise a nucleic acid sequence of:
(Human Rab1a^{S25N} ORF Codon Sequence,
SEQ ID NO: 5)
ATGTCCAGCATGAATCCCGAATATGATTATTTATTCAAGTTACTTCTGAT
TGGCGACTCAGGGGTTGGAAAGAATTGCCTTCTTCTTAGGTTTGCAGATG
ATACATATACAGAAAGCTACATCAGCACAATTGGTGTGGATTTCAAAATA
AGAACTATAGAGTTAGACGGGAAAACAATCAAGCTTCAAATATGGGACAC
AGCAGGCCAGGAAAGATTTCGAACAATCACCTCCAGTTATTACAGAGGAG
CCCATGGCATCATAGTTGTGTATGATGTGACAGATCAGGAGTCCTTCAAT
AATGTTAAACAGTGGCTGCAGGAAATAGATCGTTATGCCAGTGAAAATGT
CAACAAATTGTTGGTAGGGAACAAATGTGATCTGACCACAAAGAAAGTAG
TAGACTACACAACAGCGAAGGAATTTGCTGATTCCCTTGGAATTCCGTTT
TTGGAAACCAGTGCTAAGAATGCAACGAATGTAGAACAGTCTTTCATGAC
GATGGCAGCTGAGATTAAAAAGCGAATGGGTCCCGGAGCAACAGCTGGTG
GTGCTGAGAAGTCCAATGTTAAAATTCAGAGCACTCCAGTCAAGCA
GTCAGGTGGAGGTTGCTGCTAA;
(Mouse Rab1a^{N124I} ORF Codon Seqeunce,
SEQ ID NO: 8)
ATGGGGGACTACAAGGACGACGATGACAAGGGGGGTAGCGGTGGATCCAG
CATGAATCCCGAATATGATTATTTATTCAAGTTACTTCTGATTGGCGATT
CTGGGGTTGGAAAGTCCTGCCTTCTCCTTAGGTTTGCAGATGATACGTAT
ACGGAAAGCTACATCAGCACAATTGGTGTGGATTTCAAGATACGAACTAT
AGAGTTAGATGGGAAAACAATCAAGCTACAGATATGGGACACAGCAGGCC
AGGAAAGATTTCGAACAATCACTTCCAGTTATTACAGAGGAGCCCATGGC
ATCATAGTTGTGTATGATGTGACAGATCAGGAGTCCTTCAATAACGTTAA
ACAGTGGCTGCAGGAGATAGATCGCTACGCCAGTGAAAATGTCAACAAGT
TGTTGGTAGGGATCAAATGTGACCTGACCACAAAGAAAGTAGTAGACTAC
ACAACAGCAAAGGAATTTGCAGATTCCCTTGGAATTCCATTTTTGGAAAC
CAGTGCTAAGAACGCAACGAATGTAGAACAGTCTTTCATGACGATGGCAG
CTGAGATTAAAAAGCGAATGGGTCCTGGAGCTACAGCTGGTGGTGCCGAG
AAGTCCAATGTTAAAATCCAGAGCACTCCAGTCAAGCAGTCAGGTGGAGG
CTGCTGCTAA;
or
(Human Rab1a^{D47N} ORF Codon Sequence,
SEQ ID NO: 17)
ATGTCCAGCATGAATCCCGAATATGATTATTTATTCAAGTTACTTCTGAT
TGGCGACTCAGGGGTTGGAAAGTCTTGCCTTCTTCTTAGGTTTGCAGATG
ATACATATACAGAAAGCTACATCAGCACAATTGGTGTGAACTTCAAAATA
AGAACTATAGAGTTAGACGGGAAAACAATCAAGCTTCAAATATGGGACAC
AGCAGGCCAGGAAAGATTTCGAACAATCACCTCCAGTTATTACAGAGGAG
CCCATGGCATCATAGTTGTGTATGATGTGACAGATCAGGAGTCCTTCAAT
AATGTTAAACAGTGGCTGCAGGAAATAGATCGTTATGCCAGTGAAAATGT
CAACAAATTGTTGGTAGGGAACAAATGTGATCTGACCACAAAGAAAGTAG
TAGACTACACAACAGCGAAGGAATTTGCTGATTCCCTTGGAATTCCGTTT
TTGGAAACCAGTGCTAAGAATGCAACGAATGTAGAACAGTCTTTCATGAC
GATGGCAGCTGAGATTAAAAAGCGAATGGGTCCCGGAGCAACAGCTGGTG
GTGCTGAGAAGTCCAATGTTAAAATTCAGAGCACTCCAGTCAAGCAGTCA
GGTGGAGGTTGCTGCTAA;
or a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity therewith and encoding a Rab1a^{GDP} preferentially binding GDP;
or a nucleic acid sequence equivalent to any of the above sequences due to codon redundancy.

16. The method of claim 14, wherein the one or more expressible nucleic acids comprise a nucleic acid sequence of:
(SEQ ID NO: 19, MG-008 ORF DNA Sequence with 5′
Luciferase Tag)
ATGGAAGACGCCAAAAACATAAAGAAAGGCCCGGCGCCATTCTATCCGCT
AGAGGATGGAACCGCTGGAGAGCAACTGCATAAGGCTATGAAGAGATACG
CCCTGGTTCCTGGAACAATTGCTTTTACAGATGCACATATCGAGGTGAAC
ATCACGTACGCGGAATACTTCGAAATGTCCGTTCGGTTGGCAGAAGCTAT
GAAACGATATGGGCTGAATACAAATCACAGAATCGTCGTATGCAGTGAAA
ACTCTCTTCAATTCTTTATGCCGGTGTTGGGCGCGTTATTTATCGGAGTT
GCAGTTGCGCCCGCGAACGACATTTATAATGAACGTGAATTGCTCAACAG
TATGAACATTTCGCAGCCTACCGTAGTGTTTGTTTCCAAAAAGGGGTTGC
AAAAAATTTTGAACGTGCAAAAAAAATTACCAATAATCCAGAAAATTATT
ATCATGGATTCTAAAACGGATTACCAGGGATTTCAGTCGATGTACACGTT
CGTCACATCTCATCTACCTCCCGGTTTTAATGAATACGATTTTGTACCAG
AGTCCTTTGATCGTGACAAAACAATTGCACTGATAATGAACTCCTCTGGA
TCTACTGGGTTACCTAAGGGTGTGGCCCTTCCGCATAGAACTGCCTGCGT
CAGATTCTCGCATGCCAGAGATCCTATTTTTGGCAATCAAATCATTCCGG
ATACTGCGATTTTAAGTGTTGTTCCATTCCATCACGGTTTTGGAATGTTT
ACTACACTCGGATATTTGATATGTGGATTTCGAGTCGTCTTAATGTATAG
ATTTGAAGAAGAGCTGTTTTTACGATCCCTTCAGGATTACAAAATTCAAA
GTGCGTTGCTAGTACCAACCCTATTTTCATTCTTCGCCAAAAGCACTCTG
ATTGACAAATACGATTTATCTAATTTACACGAAATTGCTTCTGGGGGCGC
ACCTCTTTCGAAAGAAGTCGGGGAAGCGGTTGCAAAACGCTTCCATCTTC
CAGGGATACGACAAGGATATGGGCTCACTGAGACTACATCAGCTATTCTG
ATTACACCCGAGGGGGATGATAAACCGGGCGCGGTCGGTAAAGTTGTTCC
ATTTTTTGAAGCGAAGGTTGTGGATCTGGATACCGGGAAAACGCTGGGCG
TTAATCAGAGAGGCGAATTATGTGTCAGAGGACCTATGATTATGTCCGGT
TATGTAAACAATCCGGAAGCGACCAACGCCTTGATTGACAAGGATGGATG
GCTACATTCTGGAGACATAGCTTACTGGGACGAAGACGAACACTTCTTCA
TAGTTGACCGCTTGAAGTCTTTAATTAAATACAAAGGATACCAGGTGGCC
CCCGCTGAATTGGAGTCGATATTGTTACAACACCCCAACATCTTCGACGC
GGGCGTGGCAGGTCTTCCCGACGATGACGCCGGTGAACTTCCCGCCGCCG
TTGTTGTTTTGGAGCACGGAAAGACGATGACGGAAAAAGAGATCGTGGAT
TACGTCGCCAGTCAAGTAACAACCGCCAAAAAGTTGCGCGGAGGAGTTGT
GTTTGTGGACGAAGTACCGAAAGGTCTTACCGGAAAACTCGACGCAAGAA
AAATCAGAGAGATCCTCATAAAGGCCAAGAAGGGCGGAAAGTCCAAATTG
ATGTCCAGCATGAATCCCGAATATGATTATTTATTCAAGTTACTTCTGAT
TGGCGACTCAGGGGTTGGAAAGAATTGCCTTCTTCTTAGGTTTGCAGATG
ATACATATACAGAAAGCTACATCAGCACAATTGGTGTGGATTTCAAAATA
AGAACTATAGAGTTAGACGGGAAAACAATCAAGCTTCAAATATGGGACAC
AGCAGGCCAGGAAAGATTTCGAACAATCACCTCCAGTTATTACAGAGGAG
CCCATGGCATCATAGTTGTGTATGATGTGACAGATCAGGAGTCCTTCAAT
AATGTTAAACAGTGGCTGCAGGAAATAGATCGTTATGCCAGTGAAAATGT
CAACAAATTGTTGGTAGGGAACAAATGTGATCTGACCACAAAGAAAGTAG
TAGACTACACAACAGCGAAGGAATTTGCTGATTCCCTTGGAATTCCGTTT
TTGGAAACCAGTGCTAAGAATGCAACGAATGTAGAACAGTCTTTCATGAC
GATGGCAGCTGAGATTAAAAAGCGAATGGGTCCCGGAGCAACAGCTGGTG
GTGCTGAGAAGTCCAATGTTAAAATTCAGAGCACTCCAGTCAAGCAGTCA
GGTGGAGGTTGCTGCTAA;
or
(SEQ ID NO: 20, MG-008 ORF mRNA Sequence with 5′
Luciferase Tag)
AUGGAAGACGCCAAAAACAUAAAGAAAGGCCCGGCGCCAUUCUAUC
CGCUAGAGGAUGGAACCGCUGGAGAGCAACUGCAUAAGGCUAUGAAGAGA
UACGCCCUGGUUCCUGGAACAAUUGCUUUUACAGAUGCACAUAUCGAGGU
GAACAUCACGUACGCGGAAUACUUCGAAAUGUCCGUUCGGUUGGCAGAAG
CUAUGAAACGAUAUGGGCUGAAUACAAAUCACAGAAUCGUCGUAUGCAGU
GAAAACUCUCUUCAAUUCUUUAUGCCGGUGUUGGGCGCGUUAUUUAUCGG
AGUUGCAGUUGCGCCCGCGAACGACAUUUAUAAUGAACGUGAAUUGCUCA
ACAGUAUGAACAUUUCGCAGCCUACCGUAGUGUUUGUUUCCAAAAAGGGG
UUGCAAAAAAUUUUGAACGUGCAAAAAAAAUUACCAAUAAUCCAGAAAAU
UAUUAUCAUGGAUUCUAAAACGGAUUACCAGGGAUUUCAGUCGAUGUACA
CGUUCGUCACAUCUCAUCUACCUCCCGGUUUUAAUGAAUACGAUUUUGUA
CCAGAGUCCUUUGAUCGUGACAAAACAAUUGCACUGAUAAUGAACUCCUC
UGGAUCUACUGGGUUACCUAAGGGUGUGGCCCUUCCGCAUAGAACUGCCU
GCGUCAGAUUCUCGCAUGCCAGAGAUCCUAUUUUUGGCAAUCAAAUCAUU
CCGGAUACUGCGAUUUUAAGUGUUGUUCCAUUCCAUCACGGUUUUGGAAU
GUUUACUACACUCGGAUAUUUGAUAUGUGGAUUUCGAGUCGUCUUAAUGU
AUAGAUUUGAAGAAGAGCUGUUUUUACGAUCCCUUCAGGAUUACAAAAUU
CAAAGUGCGUUGCUAGUACCAACCCUAUUUUCAUUCUUCGCCAAAAGCAC
UCUGAUUGACAAAUACGAUUUAUCUAAUUUACACGAAAUUGCUUCUGGGG
GCGCACCUCUUUCGAAAGAAGUCGGGGAAGCGGUUGCAAAACGCUUCCAU
CUUCCAGGGAUACGACAAGGAUAUGGGCUCACUGAGACUACAUCAGCUAU
UCUGAUUACACCCGAGGGGGAUGAUAAACCGGGCGCGGUCGGUAAAGUUG
UUCCAUUUUUUGAAGCGAAGGUUGUGGAUCUGGAUACCGGGAAAACGCUG
GGCGUUAAUCAGAGAGGCGAAUUAUGUGUCAGAGGACCUAUGAUUAUGUC
CGGUUAUGUAAACAAUCCGGAAGCGACCAACGCCUUGAUUGACAAGGAUG
GAUGGCUACAUUCUGGAGACAUAGCUUACUGGGACGAAGACGAACACUUC
UUCAUAGUUGACCGCUUGAAGUCUUUAAUUAAAUACAAAGGAUACCAGGU
GGCCCCCGCUGAAUUGGAGUCGAUAUUGUUACAACACCCCAACAUCUUCG
ACGCGGGCGUGGCAGGUCUUCCCGACGAUGACGCCGGUGAACUUCCCGCC
GCCGUUGUUGUUUUGGAGCACGGAAAGACGAUGACGGAAAAAGAGAUCGU
GGAUUACGUCGCCAGUCAAGUAACAACCGCCAAAAAGUUGCGCGGAGGAG
UUGUGUUUGUGGACGAAGUACCGAAAGGUCUUACCGGAAAACUCGACGCA
AGAAAAAUCAGAGAGAUCCUCAUAAAGGCCAAGAAGGGCGGAAAGUCCAA
AUUGAUGUCCAGCAUGAAUCCCGAAUAUGAUUAUUUAUUCAAGUUACUUC
UGAUUGGCGACUCAGGGGUUGGAAAGAAUUGCCUUCUUCUUAGGUUUGCA
GAUGAUACAUAUACAGAAAGCUACAUCAGCACAAUUGGUGUGGAUUUCAA
AAUAAGAACUAUAGAGUUAGACGGGAAAACAAUCAAGCUUCAAAUAUGGG
ACACAGCAGGCCAGGAAAGAUUUCGAACAAUCACCUCCAGUUAUUACAGA
GGAGCCCAUGGCAUCAUAGUUGUGUAUGAUGUGACAGAUCAGGAGUCCUU
CAAUAAUGUUAAACAGUGGCUGCAGGAAAUAGAUCGUUAUGCCAGUGAAA
AUGUCAACAAAUUGUUGGUAGGGAACAAAUGUGAUCUGACCACAAAGAAA
GUAGUAGACUACACAACAGCGAAGGAAUUUGCUGAUUCCCUUGGAAUUCC
GUUUUUGGAAACCAGUGCUAAGAAUGCAACGAAUGUAGAACAGUCUUUCA
UGACGAUGGCAGCUGAGAUUAAAAAGCGAAUGGGUCCCGGAGCAACAGCU
GGUGGUGCUGAGAAGUCCAAUGUUAAAAUUCAGAGCACUCCAGUCAAGCA
GUCAGGUGGAGGUUGCUGCUAA;
or a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity therewith and encoding a Rab1a^{GDP} preferentially binding GDP;
or a nucleic acid sequence equivalent to any of the above sequences due to codon redundancy.

17.-36. (canceled)

38. A polypeptide comprising the amino acid sequence:
(SEQ ID NO: 6; Human Rab1a^{S25N})
MSSMNPEYDYLFKLLLIGDSGVGKNCLLLRFADDTYTESYISTIGVDFKI
RTIELDGKTIKLQIWDTAGQERFRTITSSYYRGAHGIIVVYDVTDQESFN
NVKQWLQEIDRYASENVNKLLVGNKCDLTTKKVVDYTTAKEFADSLGIPF
LETSAKNATNVEQSFMTMAAEIKKRMGPGATAGGAEKSNVKIQSTPVKQS
GGGCC;
(SEQ ID NO: 9; Mouse Rab1^{N1241})
MGDYKDDDDKGGSGGSSMNPEYDYLFKLLLIGDSGVGKSCLLLRFADDTY
TESYISTIGVDFKIRTIELDGKTIKLQIWDTAGQERFRTITSSYYRGAHG
IIVVYDVTDQESFNNVKQWLQEIDRYASENVNKLLVGIKCDLTTKKVVDY
TTAKEFADSLGIPFLETSAKNATNVEQSFMTMAAEIKKRMGPGATAGGAE
KSNVKIQSTPVKQSGGGCC;
the amino acid sequence of human Rab1a^{D41N}; or
(SEQ ID NO: 18; Human Rab1a^{D47N})
MSSMNPEYDYLFKLLLIGDSGVGKSCLLLRFADDTYTESYISTIGVNFKI
RTIELDGKTIKLQIWDTAGQERFRTITSSYYRGAHGIIVVYDVTDQESFN
NVKQWLQEIDRYASENVNKLLVGNKCDLTTKKVVDYTTAKEFADSLGIPF
LETSAKNATNVEQSFMTMAAEIKKRMGPGATAGGAEKSNVKIQSTPVKQS
GGGCC.
or a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity with any of these sequences and preferentially binding GDP;
for use in increasing cell viability of a dopaminergic neuron, or for preventing or treating dopaminergic neuronal death, in a subject in need thereof; or for use in increasing cell viability of a dopaminergic neuron in vitro or in vivo.

39. The polypeptide of claim 38, for use in reducing symptoms of Parkinson's disease in a subject, or for preventing or treating Parkinson's disease, in a subject in need thereof.

40. The polypeptide of claim 39, wherein the symptoms of Parkinson's disease include at least one of the following: static tremor, postural imbalance, bradykinesia, muscle rigidity, punding, dyskinesias, hallucinations, impulse control disorders and sleep disorders.

41. A pharmaceutical composition comprising
a GDP-bound form of Rab1a (Rab1a^{GDP}), one or more expressible nucleic acids encoding Rab1a^{GDP}, or a combination thereof; and
another anti-Parkinson agent.

42. A kit comprising any one or more of:
a GDP-bound form of Rab1a (Rab1a^{GDP});
one or more expressible nucleic acids encoding Rab1a^{GDP};
an anti-Parkinson agent;
instructions for performing a method as defined in claim 1; or
any combinations thereof.

43. The method of claim 3, wherein the dopaminergic neuron is located in the midbrain.

44. The method of claim 43, wherein the dopaminergic neuron is located in the substantia nigra pars compacta (SNpc) or in the substantia nigra pars reticulata (SNr).

45. A method for reducing symptoms of Parkinson's disease, or for preventing or treating Parkinson's disease, in a subject in need thereof, or for increasing cell viability of a dopaminergic neuron in vitro or in vivo, said method comprising:
treating the subject or the dopaminergic neuron with a lipid nanoparticle encapsulating a GDP-bound form of Rab1a (Rab1a^{GDP}), one or more expressible nucleic acids encoding Rab1a^{GDP}, or a combination thereof;
thereby increasing cellular levels of Rab1a^{GDP} in the subject or the dopaminergic neuron, resulting in decreased symptoms of Parkinson's disease in the subject or increased cell viability of the dopaminergic neuron.

46.-48. (canceled)
